(19) United States
(12) Patent Application Publication
(10) Pub. No.: US 2022/0081435 A1
(43) Pub. Date: Mar. 17, 2022

(54) ANDROGEN RECEPTOR PROTEIN DEGRADERS

(71) Applicant: THE REGENTS OF THE UNIVERSITY OF MICHIGAN, Ann Arbor, MI (US)

(72) Inventors: Shaomeng Wang, Superior Township, MI (US); Xin Han, Ann Arbor, MI (US); Chao Wang, Beijing (CN); Chong Qin, Ann Arbor, MI (US); Weiguo Xiang, Ypsilanti, MI (US); Tianfeng Xu, Ypsilanti, MI (US); Chao-Yie Yang, Ann Arbor, MI (US)

(21) Appl. No.: 17/420,421

(22) PCT Filed: Dec. 19, 2019
(86) PCT No.: PCT/US19/67313

§ 371 Date: Jul. 2, 2021

Related U.S. Application Data
(60) Provisional application No. 62/788,005, filed on Jan. 3, 2019.

(51) Int. Cl. C07D 417/14; A61K 47/55; A61K 45/06

(52) U.S. Cl. CPC C07D 417/14; A61K 45/06; A61K 47/55

(57) ABSTRACT

The present disclosure provides compounds represented by Formula (I): A-L-B (I), and the salts or solvates thereof, wherein A, L, and B are as defined in the specification. Compounds having Formula (I) are androgen receptor degraders useful for the treatment of cancer.

### BACKGROUND OF THE INVENTION

### Field of the Invention

The present disclosure provides heterobifunctional small molecules as androgen receptor (AR) protein degraders. AR degraders useful for the treatment of a variety of diseases including prostate cancer.

### Background

Despite improvements in medical treatments over the past three decades, prostate cancer (PCa) is significant cause of cancer-related death, and is second only to lung cancer among men in developed countries.^{1,2} In addition to surgery and radiotherapy, androgen deprivation therapies (ADT) are front-line treatments for prostate cancer patients with high-risk localized disease, and second-generation anti-androgens such as abiraterone and enzalutamide have been shown to benefit patients with advanced prostate cancer.^{3,4} Nevertheless, patients who progress to metastatic castration-resistant prostate cancer (mCRPC), a hormone-refractory form of the disease, face a high mortality rate and no cure is currently available.^{5,6}

The androgen receptor (AR) and its downstream signaling play a critical role in the development and progression of both localized and metastatic prostate cancer.⁷ Previous strategies that successfully target AR signaling have focused on blocking androgen synthesis by drugs such as abiraterone and inhibition of AR function by AR antagonists such as enzalutamide and apalutamide (ARN-509).⁸⁻¹⁴ However, such agents become ineffective in advanced prostate cancer with AR gene amplification, mutation and alternate splicing.^{15,16} But in most patients with CRPC, the AR protein continues to be expressed and tumors are still dependent upon AR signaling. Consequently, AR is an attractive therapeutic target for mCRPC.^{17,18}

The Proteolysis Targeting Chimera (PROTAC) strategy has gained momentum with its promise in the discovery and development of completely new types of small molecule therapeutics by inducing targeted protein degradation.¹⁹⁻²⁵ A PROTAC molecule is a heterobifunctional small molecule containing one ligand, which binds to the target protein of interest, and a second ligand for an E3 ligase system, tethered together by a chemical linker.²⁶ Because AR protein plays a key role in CRPC, AR degraders designed based upon the PROTAC concept could be effective for the treatment of CRPC when the disease becomes resistant to AR antagonists or to androgen synthesis inhibitors.²⁷⁻³⁰ Naito et al. have recently reported AR degraders designed based upon the PROTAC concept, which were named Specific and Nongenetic LAP-dependent Protein Erasers (SNIPERs).³¹ An AR SNIPER molecule, for example compound 1:

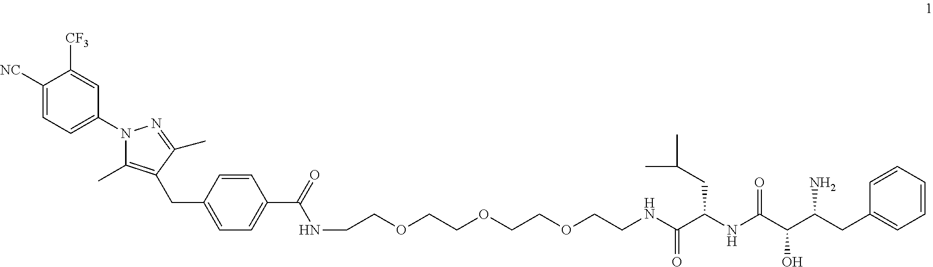 [SMILES: Cc1nn(-c2ccc(C#N)c(C(F)(F)F)c2)c(C)c1Cc1ccc(C(=O)NCCOCCOCCOCCNC(=O)[C@H](CC(C)C)NC(=O)[C@@H](O)[C@H](N)Cc2ccccc2)cc1]

(chemical name: N-((14S,17S,18R)-18-amino-17-hydroxy-14-isobutyl-13,16-dioxo-19-phenyl-3,6,9-trioxa-12,15-diazanonadecyl)-4-((1-(4-cyano-3-(trifluoromethyl)phenyl)-3,5-dimethyl-1H-pyrazol-4-yl)methyl)benzamide) was designed using an AR antagonist and a ligand for the cellular inhibitor of apoptosis protein 1 (cIAP1) as the E3 ligase. While SNIPER AR degraders are effective in inducing partial degradation of the AR protein in cells, they also induce the auto-ubiquitylation and proteasomal degradation of the cIAP1 protein, the E3 ligase needed for induced degradation of AR protein, thus limiting their AR degradation efficiency and therapeutic efficacy.

Compound 2 (ARCC-4):

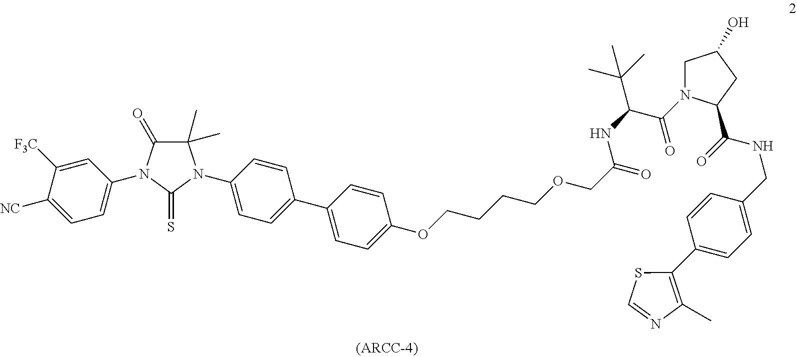 [SMILES: Cc1ncsc1-c1ccc(CNC(=O)[C@@H]2C[C@@H](O)CN2C(=O)[C@@H](NC(=O)COCCCCOc2ccc(-c3ccc(N4C(=S)N(c5ccc(C#N)c(C(F)(F)F)c5)C(=O)C4(C)C)cc3)cc2)C(C)(C)C)cc1]

(chemical name: (4R)-1-((S)-2-(2-(4-((4′-(3-(4-cyano-3-(trifluoromethyl)phenyl)-5,5-dimethyl-4-oxo-2-thioxoimidazolidin-1-yl)-[1,1′-biphenyl]-4-yl)oxy)butoxy)acetamido)-3,3-dimethylbutanoyl)-4-hydroxy-N-(4-(4-methylthiazol-5-yl)benzyl)pyrrolidine-2-carboxamide) was recently reported as another PROTAC degrader, which was designed using enzalutamide as the AR antagonist and a von Hippel-Lindau (VHL) ligand.^{27,32} ARCC-4 was shown to be more potent and effective than enzalutamide at inducing apoptosis and inhibiting proliferation of AR-amplified prostate cancer cells.^{27,32}

There is a need in the art for additional AR degraders to treat prostate cancer and other diseases.

### BRIEF SUMMARY OF THE INVENTION

In one aspect, the present disclosure provides heterobifunctional small molecules represented by any one or more of Formulae I-V, below, and the pharmaceutically acceptable salts and solvates, e.g., hydrates, thereof, collectively referred to herein as “Compounds of the Disclosure.” Compounds of the Disclosure are androgen receptor degraders and are thus useful in treating diseases or conditions wherein degradation of the androgen receptor provides a therapeutic benefit to a patient.

In another aspect, the present disclosure provides methods of treating a condition or disease by administering a therapeutically effective amount of a Compound of the Disclosure to a patient, e.g., a human, in need thereof. The disease or condition is treatable by degradation of the androgen receptor, for example, a cancer, e.g., prostate cancer.

In another aspect, the present disclosure provides a method of degrading of the androgen receptor in an individual, comprising administering to the individual an effective amount of at least one Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating diseases or conditions wherein degradation of the androgen receptor provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in treatment of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In another aspect, the present disclosure provides methods of preparing Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims. It is to be understood that both the foregoing summary and the following detailed description are exemplary and explanatory only, and are not restrictive of the invention as claimed.

### BRIEF DESCRIPTION OF DRAWINGS

FIG. 1 is an image of a Western blotting analysis of AR protein in LNCaP cells treated with AR degrader 34 (ARD-69), with GAPDH used as the loading control. Cells were treated with 100 nM of ARD-69 for indicated time points.

FIG. 2 is an image of an Western blotting analysis of AR protein in VCaP cells treated with AR degrader 34 (ARD-69), with GAPDH used as the loading control. Cells were treated with 100 nM of ARD-69 for indicated time points.

FIG. 3 is a line graph showing the dose-dependent AR degradation by 34 (ARD-69) in VCaP cells. Cells were treated for 24 h.

FIG. 4 is a line graph showing the dose-dependent AR degradation by 34 (ARD-69) in LNCaP cells. Cells were treated for 24 h.

FIG. 5 is a line graph showing the dose-dependent AR degradation by 34 (ARD-69) in 22RV1 cells. Cells were treated for 24 h.

FIG. 6 is a line graph showing the cell growth inhibition in LNCaP cells treated with AR degrader 34 (ARD-69) and two AR antagonists enzalutamide (4) and 6. LNCaP were treated with different compounds in charcoal stripped medium in the presence of 0.1 nM of AR agonist R1881 for 7 days. Cell viability was determined by a WST-8 assay.

FIG. 7 is a line graph showing the cell growth inhibition in VCaP cells treated with AR degrader 34 (ARD-69) and two AR antagonists enzalutamide (4) and 6. VCaP were treated with different compounds in charcoal stripped medium in the presence of 0.1 nM of AR agonist R1881 for 7 days. Cell viability was determined by a WST-8 assay.

FIG. 8 is a line graph showing the cell growth inhibition in 22RV1 cells treated with AR degrader 34 (ARD-69) and two AR antagonists enzalutamide (4) and 6. 22RV1 cells were treated with a regular culture medium for 7 days. Cell viability was determined by a WST-8 assay.

FIG. 9 is an image of an immunoblotting analysis of a pharmacodynamics (PD) study of AR degrader 34 (ARD-69) in VCaP tumor tissue in mice. SCID mice bearing xenograft VCaP tumors were treated with a single dose of 34 (ARD-69) (IP, 50 mg/kg). Tumor tissues were harvested at the indicated time points for immunoblotting.

### DETAILED DESCRIPTION OF THE INVENTION

### I. Compounds of the Disclosure

Compounds of the Disclosure are heterobifunctional AR receptor degraders. In one embodiment, Compounds of the Disclosure are compounds represented by Formula I:

A-L-B

wherein:

A is a radical of an androgen receptor antagonist selected from the group consisting of:

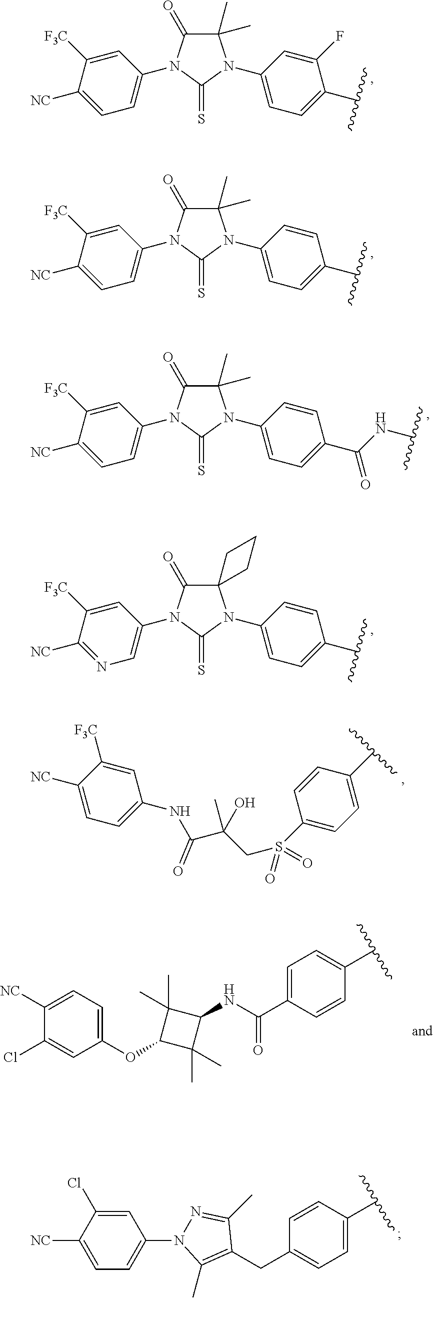

L is a linker; and

B is a radical of an E3 ligase ligand selected from the group consisting of:

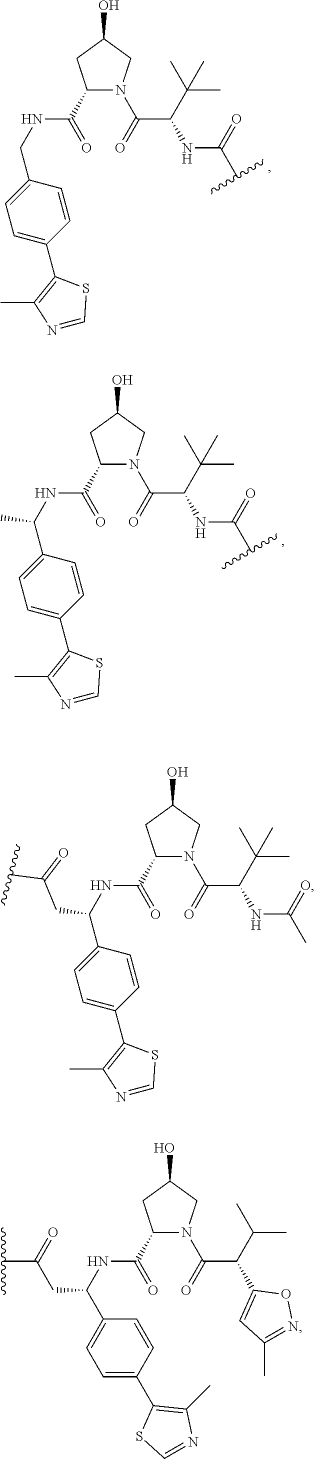

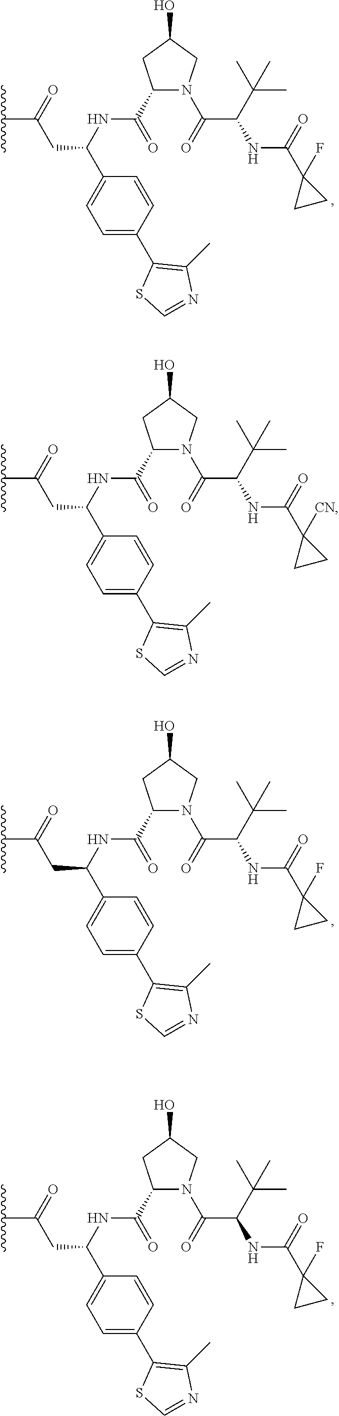

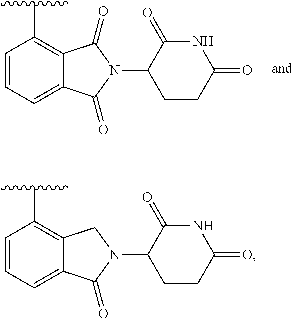

or a pharmaceutically acceptable salt or solvate thereof,

with the proviso the compound of Formula I is not (4R)-1-((S)-2-(2-(4-((4′-(3-(4-cyano-3-(trifluoromethyl)phenyl)-5,5-dimethyl-4-oxo-2-thioxoimidazolidin-1-yl)-[1,1′-biphenyl]-4-yl)oxy)butoxy)acetamido)-3,3-dimethylbutanoyl)-4-hydroxy-N-(4-(4-methylthiazol-5-yl)benzyl)pyrrolidine-2-carboxamide.

In another embodiment, Compounds of the Disclosure are compounds represented by Formula I, wherein the radical of an androgen receptor antagonist is selected from the group consisting of:

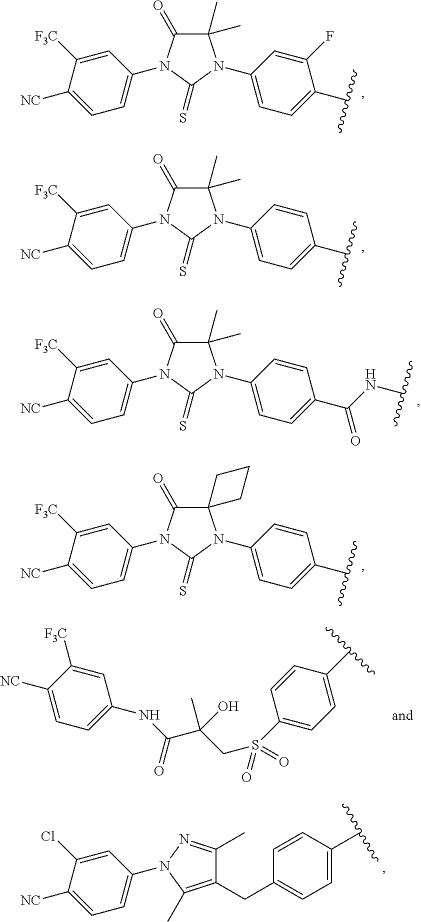

or a pharmaceutically acceptable salt or solvate thereof.

In another embodiment, Compounds of the Disclosure are compounds represented by Formula I, wherein the radical of the androgen receptor antagonist is:

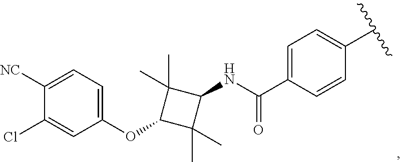

or a pharmaceutically acceptable salt or solvate thereof.

In another embodiment, Compounds of the Disclosure are compounds represented by Formula I, wherein the radical of an E3 ligase ligand is selected from the group consisting of:

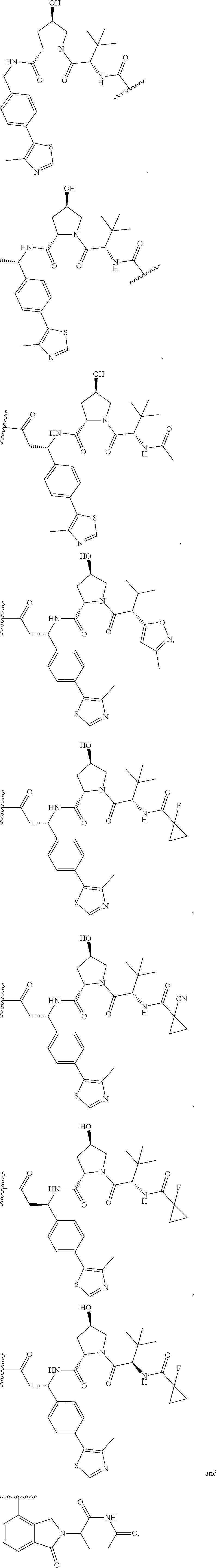

or a pharmaceutically acceptable salt or solvate thereof.

In another embodiment, Compounds of the Disclosure are compounds represented by Formula II:

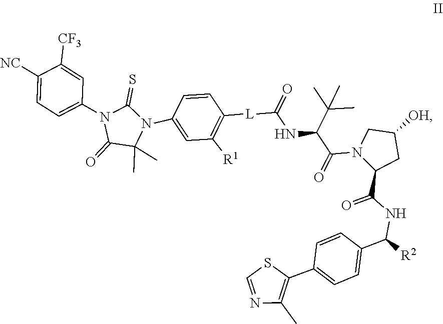

wherein:

R¹ is selected from the group consisting of hydrogen and fluoro; and

R² is selected from the group consisting of hydrogen and C₁-C₃ alkyl; and

L is as defined in connection with Formula I, or a pharmaceutically acceptable salt or solvate thereof.

In another embodiment, Compounds of the Disclosure are compounds represented by Formula III:

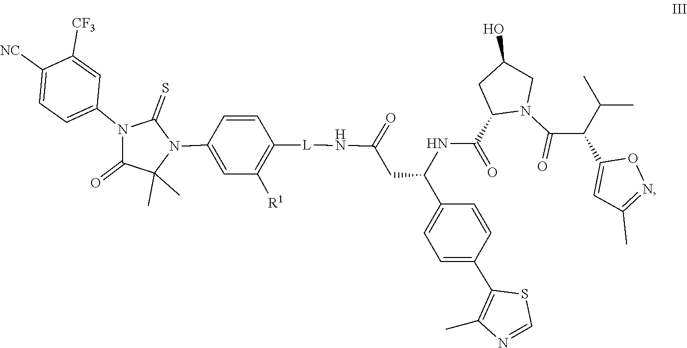

wherein:

R¹ is selected from the group consisting of hydrogen and fluoro; and

L is as defined in connection with Formula I, or a pharmaceutically acceptable salt or solvate thereof.

In another embodiment, Compounds of the Disclosure are compounds represented by any one of Formulae I-III, wherein:

L is —X-L¹-Z—;

X is selected from the group consisting of —C≡C—, —O—, —C(═O)N(R³)—, and —N(R⁴)—; or

X is absent;

Z is selected from the group consisting of —C≡C—, —O—, —C(═O)N(R³)—, and —N(R⁴)—; or

Z is absent;

L¹ is selected from the group consisting of alkylenyl, heteroalkylenyl, and —W¹—(CH₂)ₘ—W²—(CH₂)ₙ—

W¹ is absent; or

W¹ is selected from the group consisting of phenylenyl, heteroarylenyl, heterocyclenyl, and cycloalkylenyl;

W² is selected from the group consisting of phenylenyl, heteroarylenyl, heterocyclenyl, and cycloalkylenyl;

m is 0, 1, 2, 3, 4, 5, 6, or 7;

n is 0, 1, 2, 3, 4, 5, 6, 7, or 8; and

R³ is selected from the group consisting of hydrogen and C₁₋₄ alkyl; and

R⁴ is selected from the group consisting of hydrogen and C₁₋₄ alkyl,

or a pharmaceutically acceptable salt or solvate thereof.

In another embodiment, Compounds of the Disclosure are compounds represented by any one of Formulae I-III, wherein L is selected from the group consisting of:

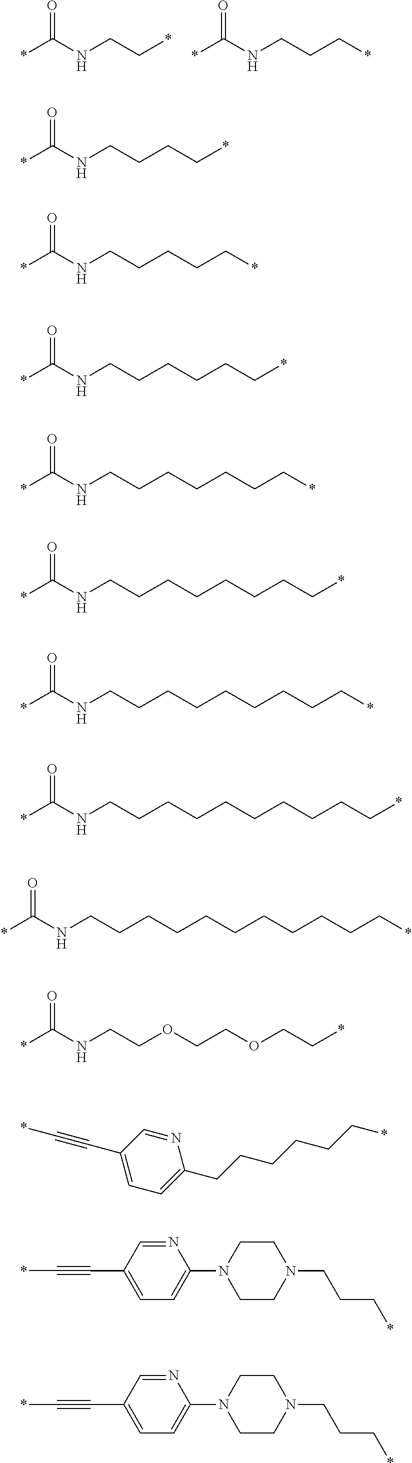

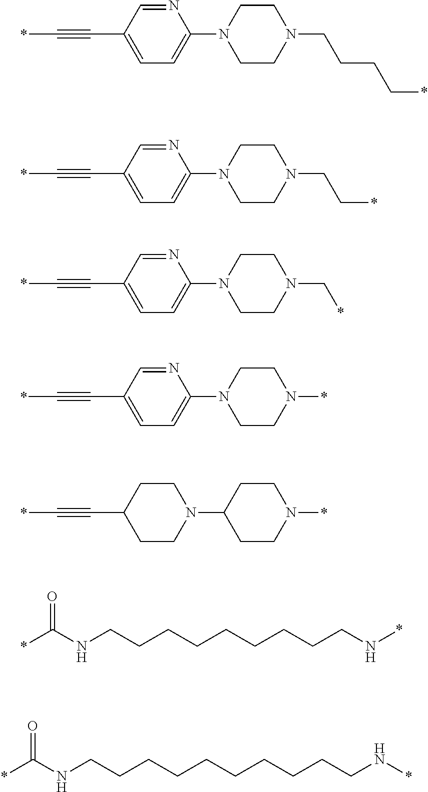

or a pharmaceutically acceptable salt or solvate thereof.

In another embodiment, Compounds of the Disclosure are compounds represented by Formula IV:

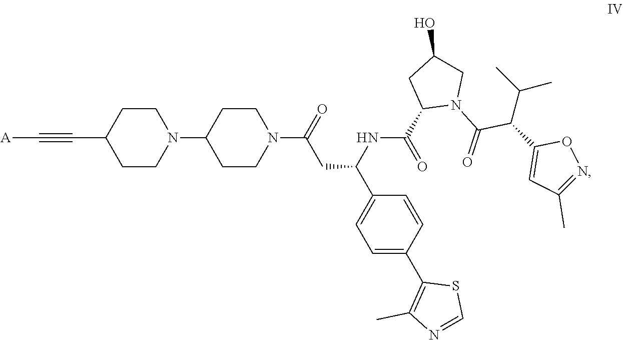

wherein A is as defined in connection with Formula I, or a pharmaceutically acceptable salt or solvate thereof.

In another embodiment, Compounds of the Disclosure are compounds represented by Formula IV, wherein A is selected from the group consisting of:

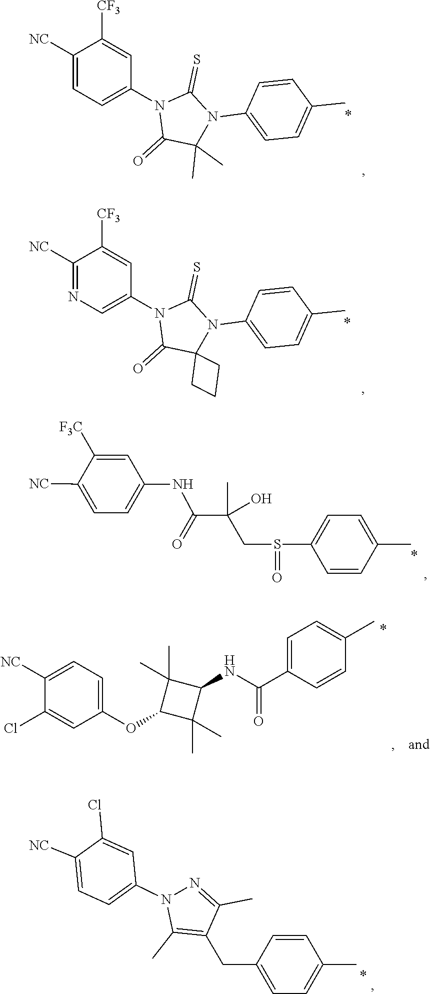 [SMILES: Cc1ccc(N2C(=S)N(c3ccc(C#N)c(C(F)(F)F)c3)C(=O)C2(C)C)cc1 ; Cc1ccc(N2C(=S)N(c3cnc(C#N)c(C(F)(F)F)c3)C(=O)C23CCC3)cc1 ; Cc1ccc(S(=O)CC(C)(O)C(=O)Nc2ccc(C#N)c(C(F)(F)F)c2)cc1 ; Cc1ccc(C(=O)N[C@H]2C(C)(C)[C@H](Oc3ccc(C#N)c(Cl)c3)C2(C)C)cc1 ; Cc1ccc(Cc2c(C)nn(-c3ccc(C#N)c(Cl)c3)c2C)cc1]

or a pharmaceutically acceptable salt or solvate thereof.

In another embodiment, Compounds of the Disclosure are compounds represented by Formula V:

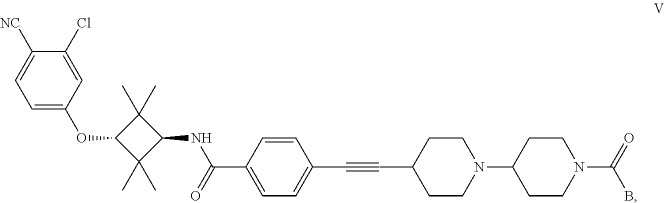

wherein B is as defined in connection with Formula I, or a pharmaceutically acceptable salt or solvate thereof.

In another embodiment, Compounds of the Disclosure are compounds represented by Formula V, wherein B is selected from the group consisting of:

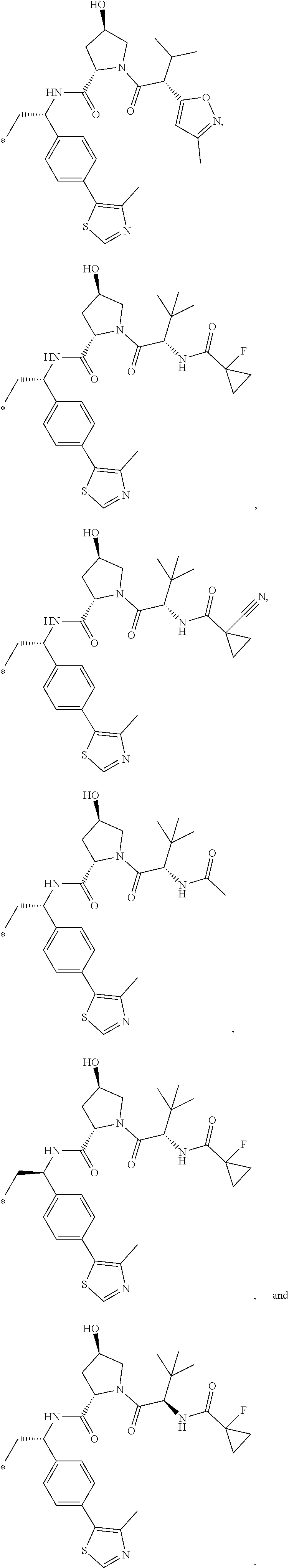

or a pharmaceutically acceptable salt or solvate thereof.

In another embodiment, Compounds of the Disclosure are compounds having Formulae I-III, and the salts or solvates thereof, wherein X is —C≡C—.

In another embodiment, Compounds of the Disclosure are compounds having Formulae I-III, and the salts or solvates thereof, wherein X is —N(H)—.

In another embodiment, Compounds of the Disclosure are compounds having Formulae I-III, and the salts or solvates thereof, wherein W¹ is

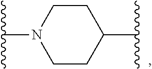 [SMILES: *C1CCN(*)CC1]

and the carbon atom of

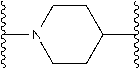 [SMILES: *C1CCN(*)CC1]

is attached to L¹.

In another embodiment, Compounds of the Disclosure are compounds having Formulae I-III, and the salts or solvates thereof, wherein L is C₁₋₁₂ alkylenyl.

In another embodiment, Compounds of the Disclosure are compounds having Formulae I-III, and the salts or solvates thereof, wherein L is selected from the group consisting of —CH₂—, —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, and —CH₂(CH₂)₆CH₂—.

In another embodiment, Compounds of the Disclosure are compounds having Formula I, and the salts or solvates thereof, wherein L is 3- to 12-membered heteroalkylenyl.

In another embodiment, Compounds of the Disclosure are compounds having Formulae I-III, and the salts or solvates thereof, wherein L is -A-(CH₂)ₘ—W—(CH₂)ₙ— and A is absent.

In another embodiment, Compounds of the Disclosure are compounds having Formulae I-III, and the salts or solvates thereof, wherein L is selected from the group consisting of:

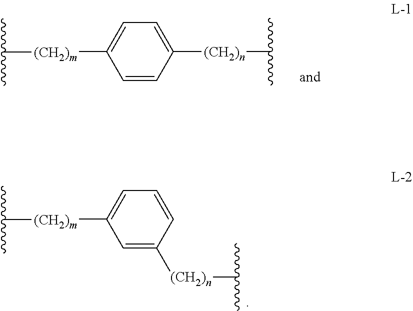

In another embodiment, Compounds of the Disclosure are compounds having Formulae I-III, and the salts or solvates thereof, wherein L is -A-(CH₂)ₘ—W—(CH₂)ₙ—, A is absent, and W is 5-membered heteroarylenyl. In another embodiment, m is 0.

In another embodiment, Compounds of the Disclosure are compounds having Formulae I-III, and the salts or solvates thereof, wherein:

L is selected from the group consisting of:

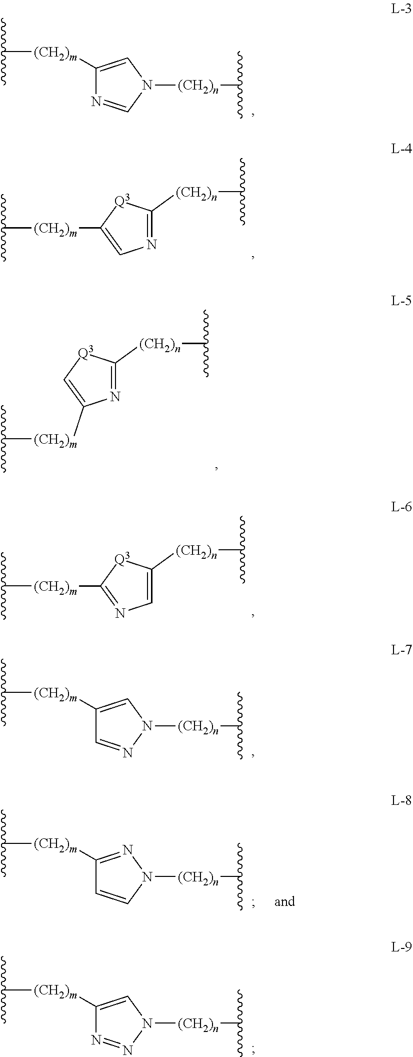

Q³ is selected from the group consisting of —O—, —S—, and —N(R⁶)—; and

R⁶ is selected from the group consisting of hydrogen and C₁₋₄ alkyl.

In another embodiment, Compounds of the Disclosure are compounds having Formulae I-III, and the salts or solvates thereof, wherein L is -A-(CH₂)ₘ—W—(CH₂)ₙ—, A is absent, and W is 6-membered heteroarylenyl. In another embodiment, m is 0.

In another embodiment, Compounds of the Disclosure are compounds having Formulae I-III, and the salts or solvates thereof, wherein L is selected from the group consisting of:

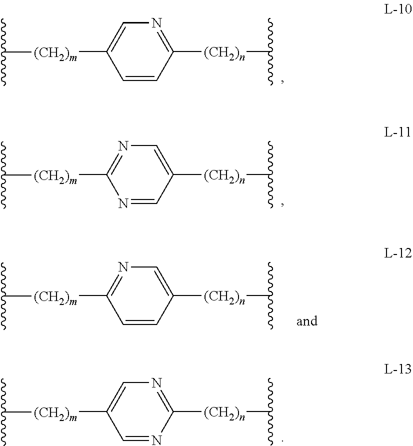

In another embodiment, Compounds of the Disclosure are compounds having Formulae I-III, and the salts or solvates thereof, wherein L is -A-(CH₂)ₘ—W—(CH₂)ₙ—, A is absent, and W is heterocyclenyl. In another embodiment, m is 0.

In another embodiment, Compounds of the Disclosure are compounds having Formulae I-III, and the salts or solvates thereof, wherein L is selected from the group consisting of:

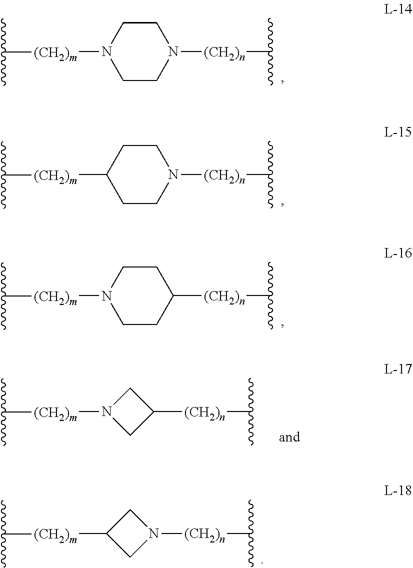

In another embodiment, Compounds of the Disclosure are compounds having Formulae I-III, and the salts or solvates thereof, wherein L is selected from the group consisting of:

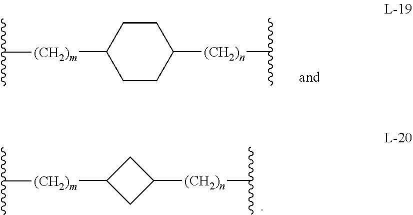

In another embodiment, Compounds of the Disclosure are compounds having Formulae I-III, and the salts or solvates thereof, wherein L is selected from the group consisting of:

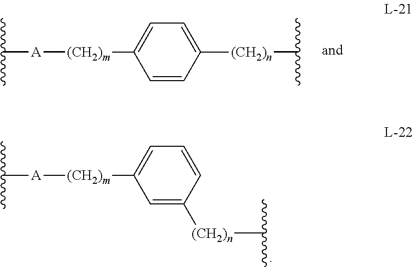

In another embodiment, Compounds of the Disclosure are compounds having Formulae I-III, and the salts or solvates thereof, wherein L is selected from the group consisting of:

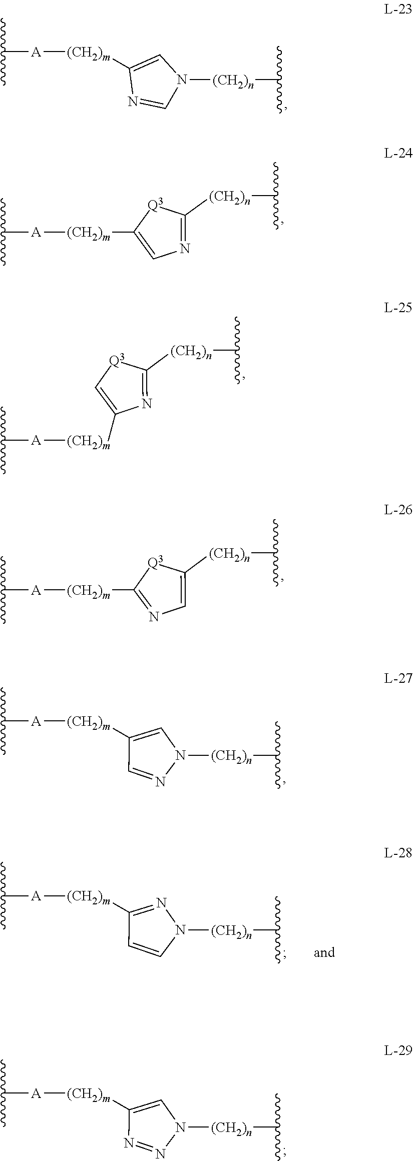

Q³ is selected from the group consisting of —O—, —S—, and —N(R⁶)—; and

R⁶ is selected from the group consisting of hydrogen and C₁₋₄ alkyl.

In another embodiment, Compounds of the Disclosure are compounds having Formulae I-III, and the salts or solvates thereof, wherein L is selected from the group consisting of:

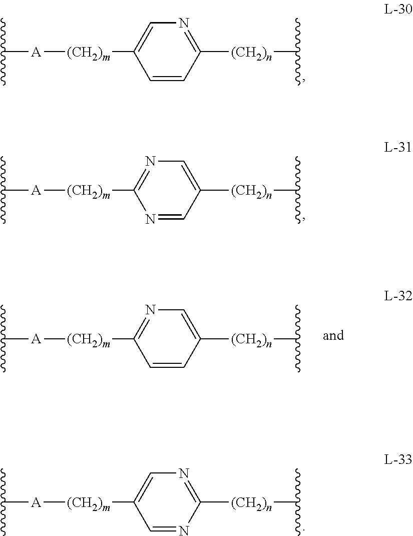

In another embodiment, Compounds of the Disclosure are compounds having Formulae I-III, and the salts or solvates thereof, wherein L is selected from the group consisting of:

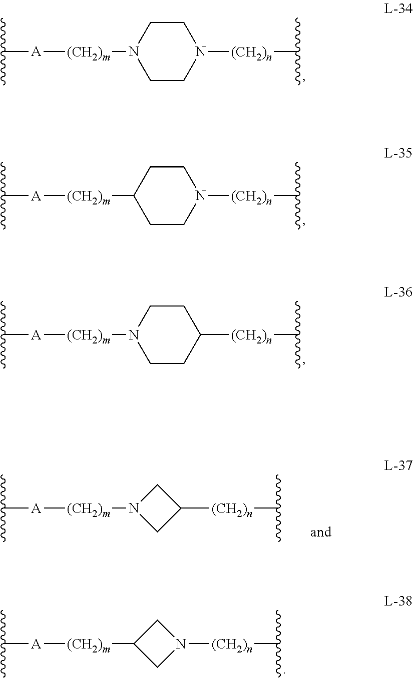

In another embodiment, Compounds of the Disclosure are compounds having Formulae I-III, and the salts or solvates thereof, wherein L is selected from the group consisting of:

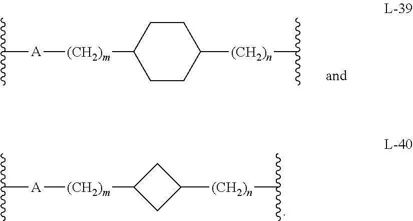

Salts, hydrates, and solvates of the Compounds of the Disclosure can also be used in the methods disclosed herein. The present disclosure further includes all possible stereoisomers and geometric isomers of Compounds of the Disclosure to include both racemic compounds and optically active isomers. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure and the heterobifunctional target protein degraders prepared from Compounds of the Disclosure, including pharmaceutically acceptable salts. As used herein, the pharmaceutical “pharmaceutically acceptable salt” refers to salts or zwitterionic forms of Compounds of the Disclosure and the heterobifunctional target protein degraders prepared from Compounds of the Disclosure. Salts of Compounds of the Disclosure and the heterobifunctional target protein degraders prepared from Compounds of the Disclosure can be prepared during the final isolation and purification of the compounds or separately by reacting the compound with an acid having a suitable cation. The pharmaceutically acceptable salts of Compounds of the Disclosure and the heterobifunctional target protein degraders prepared from Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure and the heterobifunctional target protein degraders prepared from Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure and the heterobifunctional target protein degraders prepared from Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Therapeutic Methods of the Disclosure

Compounds of the Disclosure degrade AR protein and are useful in the treatment of a variety of diseases and conditions. In particular, Compounds of the Disclosure are useful in methods of treating a disease or condition wherein degradation AR proteins provides a benefit, for example, cancers and proliferative diseases. The therapeutic methods of the disclosure comprise administering a therapeutically effective amount of a Compound of the Disclosure to an individual in need thereof. The present methods also encompass administering a second therapeutic agent to the individual in addition to the Compound of the Disclosure. The second therapeutic agent is selected from drugs known as useful in treating the disease or condition afflicting the individual in need thereof, e.g., a chemotherapeutic agent and/or radiation known as useful in treating a particular cancer.

The present disclosure provides Compounds of the Disclosure as AR protein degraders for the treatment of a variety of diseases and conditions wherein degradation of AR proteins has a beneficial effect. Compounds of the Disclosure typically have a binding affinity (IC₅₀) to AR of less than 100 μM, e.g., less than 50 μM, less than 25 μM, and less than 5 μM, less than about 1 μM, less than about 0.5 μM, or less than about 0.1 μM. In one embodiment, the present disclosure relates to a method of treating an individual suffering from a disease or condition wherein degradation of AR proteins provides a benefit comprising administering a therapeutically effective amount of a Compound of the Disclosure to an individual in need thereof.

Since Compounds of the Disclosure are degraders of AR protein, a number of diseases and conditions mediated by AR can be treated by employing these compounds. The present disclosure is thus directed generally to a method for treating a condition or disorder responsive to degradation of R in an animal, e.g., a human, suffering from, or at risk of suffering from, the condition or disorder, the method comprising administering to the animal an effective amount of one or more Compounds of the Disclosure.

The present disclosure is further directed to a method of degrading AR protein in an animal in need thereof, said method comprising administering to the animal an effective amount of at least one Compound of the Disclosure.

The methods of the present disclosure can be accomplished by administering a Compound of the Disclosure as the neat compound or as a pharmaceutical composition. Administration of a pharmaceutical composition, or neat compound of a Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a Compound of the Disclosure and, optionally, a second therapeutic agent useful in the treatment of diseases and conditions wherein degradation of AR protein provides a benefit, packaged separately or together, and an insert having instructions for using these active agents.

In one embodiment, a Compound of the Disclosure is administered in conjunction with a second therapeutic agent useful in the treatment of a disease or condition wherein degradation of AR protein provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered from a single composition or two separate compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more doses of the second therapeutic agent can be administered. The Compound of the Disclosure therefore can be used in conjunction with one or more second therapeutic agents, for example, but not limited to, anticancer agents.

Diseases and conditions treatable by the methods of the present disclosure include, but are not limited to, cancer and other proliferative disorders. In one embodiment, a human patient is treated with a Compound of the Disclosure, or a pharmaceutical composition comprising a Compound of the Disclosure, wherein the compound is administered in an amount sufficient to degrade AR protein in the patient.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by degrading AR protein. In one embodiment, the cancer is prostate cancer.

In methods of the present disclosure, a therapeutically effective amount of a Compound of the Disclosure, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the AR protein degrader that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate. Buffers and pH modifiers can also be added to stabilize the pharmaceutical composition.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

### III. Definitions

The term “androgen receptor antagonist” as used herein refers to a class of drugs that prevent androgens, e.g., testosterone and dihydrotestosterone (DHT), from mediating their biological effects in the body. Representative androgen receptor antagonists include, but are not limited to:

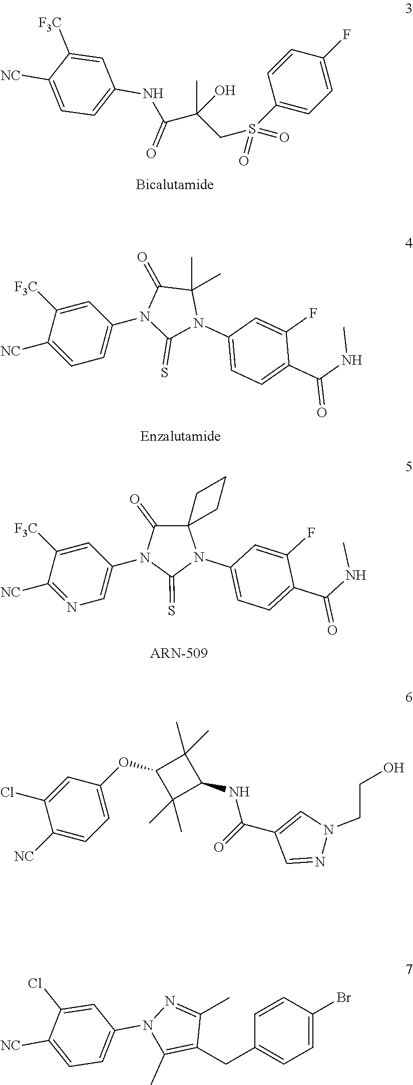

The term “radical of an androgen receptor antagonist” as used herein refers to the chemical species wherein an atom, e.g., hydrogen, or group of atoms, e.g., —C(═O)N(H)CH₃, from a parent androgen receptor antagonist is missing. For example, removal of —C(═O)N(H)CH₃ from enzalutamide (4) gives the following radical of an androgen receptor antagonist:

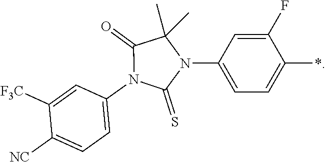

The absence of a hydrogen atom or group of atoms provides for the linkage of the parent androgen receptor antagonist to an E3 ubiquitin ligase protein ligand to give a heterobifunctional compound having Formula I as defined above.

The term “E3 ligase ligand” as herein refers to a compound that binds, e.g., inhibits, an E3 ubiquitin ligase protein, including the von Hippel-Lindau protein (VHL). Ligands for E3 ubiquitin ligase proteins are known to those of ordinary skill in the art. Exemplary non-limiting ligands for an E3 ubiquitin ligase protein include phthalimide-based drugs such as thalidomide or a VHL ligand including, but not limited to, the VHL ligands of Chart 1.

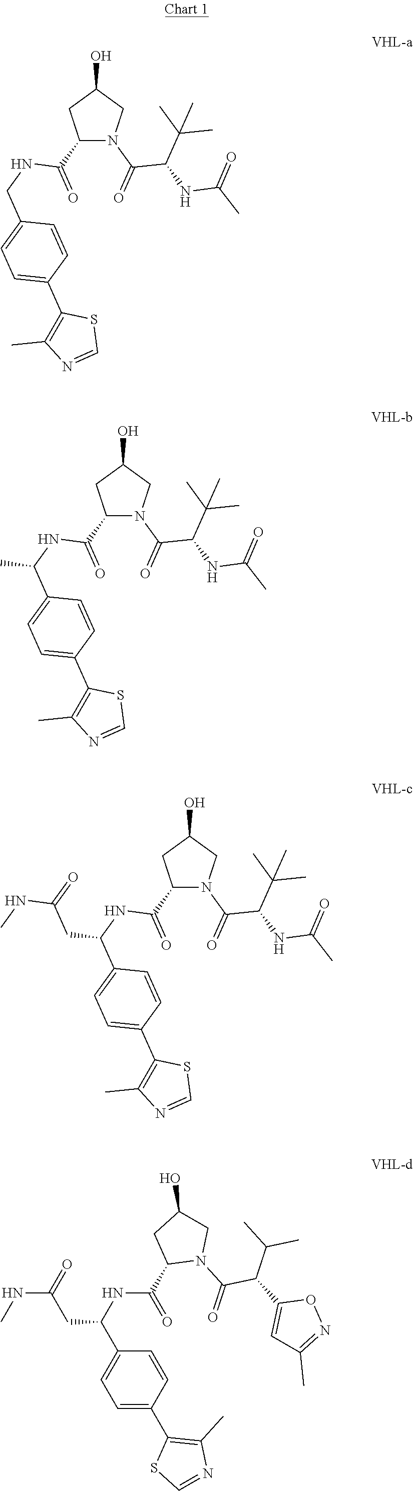

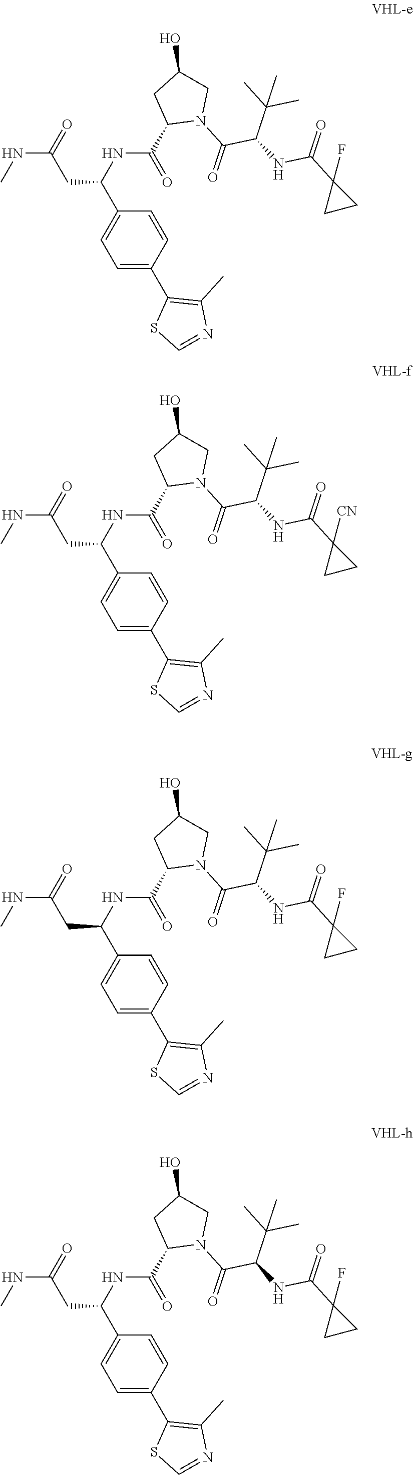 [SMILES: CNC(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; CNC(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1 ; CNC(=O)C[C@@H](NC(=O)[C@@H]1C[C@@H](O)CN1C(=O)[C@@H](NC(=O)C1(F)CC1)C(C)(C)C)c1ccc(-c2scnc2C)cc1 ; CNC(=O)C[C@H](NC(=O)[C@@H]1C[C@@H](O)CN1C(=O)[C@H](NC(=O)C1(F)CC1)C(C)(C)C)c1ccc(-c2scnc2C)cc1]

A fluorescence-polarization (FP)-based binding assay for VHL protein was developed and used to determine the binding affinities of the VHL ligands of Chart 1.

The phrase “radical of an E3 ligase ligand” refers to chemical species wherein an atom, e.g., hydrogen, or group of atoms, e.g., —CH₃, from a parent E3 ligase ligand is missing. For example, removal of —CH₃ from VHL-a, see above, gives the following radical of an E3 ligase ligand:

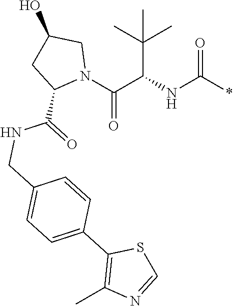

The absence of hydrogen in thalidomide gives the following radical of an E3 ligase ligand:

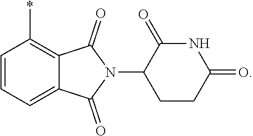

The absence of a hydrogen atom or group of atoms provides the linkage of the parent E3 ligase ligand to an androgen receptor antagonist to give a heterobifunctional compound having Formula I as defined above.

The term “linker” as used herein refers to a divalent chemical moiety capable of tethering a radical of an androgen receptor antagonist to a radical of an E3 ligase ligand.

The term “about,” as used herein, includes the recited number f 10%. Thus, “about 10” means 9 to 11.

In the present disclosure, the term “halo” as used by itself or as part of another group refers to —Cl, —F, —Br, or —I.

In the present disclosure, the term “nitro” as used by itself or as part of another group refers to —NO₂.

In the present disclosure, the term “cyano” as used by itself or as part of another group refers to —CN.

In the present disclosure, the term “hydroxy” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₂₀ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl is a C₁₋₄ alkyl. In another embodiment, the alkyl is a C₁₋₆ alkyl. In another embodiment, the alkyl is a C₁₋₄ alkyl. In another embodiment, the alkyl is a straight chain C₁₋₁₀ alkyl. In another embodiment, the alkyl is a branched chain C₃₋₁₀ alkyl. In another embodiment, the alkyl is a straight chain C₁₋₆ alkyl. In another embodiment, the alkyl is a branched chain C₃₋₆ alkyl. In another embodiment, the alkyl is a straight chain C₁₋₄ alkyl. In another embodiment, the alkyl is a branched chain C₃₋₄ alkyl. In another embodiment, the alkyl is a straight or branched chain C₃₋₄alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “heteroalkyl” as used by itself or part of another group refers to unsubstituted straight- or branched-chain aliphatic hydrocarbons containing from three to thirty chain atoms, i.e., 3- to 30-membered heteroalkyl, or the number of chain atoms designated, wherein at least one —CH₂— is replaced with at least one —O—, —N(H)—, or —S—. The —O—, N(H)—, or —S— can independently be placed at any interior position of the aliphatic hydrocarbon chain so long as each —O—, N(H)—, or —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. In another embodiment, four —CH₂— groups are replaced with four —O— groups. Non-limiting exemplary heteroalkyl groups include:

—CH₂OCH₃;

—CH₂OCH₂CH₂CH₃;

—CH₂CH₂CH₂OCH₃;

—CH₂OCH₂CH₂OCH₃; and

—CH₂OCH₂CH₂OCH₂CH₂OCH₃.

In the present disclosure, the term “alkylenyl” as used herein by itself or part of another group refers to a divalent form of an alkyl group. In one embodiment, the alkylenyl is a divalent form of a C₁₋₁₂ alkyl. In one embodiment, the alkylenyl is a divalent form of a C₁₋₁₀ alkyl. In one embodiment, the alkylenyl is a divalent form of a C₁₋₈ alkyl. In one embodiment, the alkylenyl is a divalent form of a C₁₋₆ alkyl. In another embodiment, the alkylenyl is a divalent form of a C₁₋₄ alkyl. Non-limiting exemplary alkylenyl groups include:

—CH₂—,

—CH₂CH₂—,

—CH₂CH₂CH₂—,

—CH₂(CH₂)₂CH₂—,

—CH(CH₂)₃CH₂—,

—CH₂(CH₂)₄CH₂—,

—CH₂(CH₂)₅CH₂—,

—CH₂CH(CH₃)CH₂—, and

—CH₂C(CH₃)₂CH₂—.

In the present disclosure, the term “heteroalkylenyl” as used herein by itself or part of another group refers to a divalent form of a heteroalkyl group. In one embodiment, the heteroalkylenyl is a divalent form of a 3- to 12-membered heteroalkyl. In another embodiment, the heteroalkylenyl is a divalent form of a 3- to 10-membered heteroalkyl. In another embodiment, the heteroalkylenyl is a divalent form of a 3- to 8-membered heteroalkyl. In another embodiment, the heteroalkylenyl is a divalent form of a 3- to 6-membered heteroalkyl. In another embodiment, the heteroalkylenyl is a divalent form of a 3- to 4-membered heteroalkyl. In another embodiment, the heteroalkylenyl is a radical of the formula: —(CH₂)ₒO—(CH₂CH₂O)ₚ—(CH₂)_{q}—, wherein o is 2 or 3; p is 0, 1, 2, 3, 4, 5, 6, or 7; and q is 2 or 3. In another embodiment, the heteroalkylenyl is a radical of the formula: —(CH₂)ᵣO—(CH₂)ₛ—O(CH₂)ᵣ, wherein r is 2, 3, or 4; s is 3, 4, or 5; and t is 2 or 3. Non-limiting exemplary heteroalkylenyl groups include:

—CH₂OCH₂—;

—CH₂CH₂OCH₂CH₂—;

—CH₂OCH₂CH₂CH₂—;

—CH₂CH₂OCH₂CH₂CH₂—;

—CH₂CH₂OCH₂CH₂OCH₂CH₂—; and

—CH₂CH₂OCH₂CH₂OCH₂CH₂O—.

In the present disclosure, the term “optionally substituted alkyl” as used by itself or as part of another group means that the alkyl as defined above is either unsubstituted or substituted with one, two, or three substituents independently chosen from nitro, haloalkoxy, aryloxy, aralkyloxy, alkylthio, sulfonamido, alkylcarbonyl, arylcarbonyl, alkylsulfonyl, arylsulfonyl, carboxy, carboxyalkyl, cycloalkyl, and the like. In one embodiment, the optionally substituted alkyl is substituted with two substituents. In another embodiment, the optionally substituted alkyl is substituted with one substituent. Non-limiting exemplary optionally substituted alkyl groups include —CH₂CH₂NO₂, —CH₂SO₂CH₃ CH₂CH₂CO₂H, —CH₂CH₂SO₂CH₃, —CH₂CH₂COPh, and —CH₂C₆H₁₁.

In the present disclosure, the term “cycloalkyl” as used by itself or as part of another group refers to saturated and partially unsaturated (containing one or two double bonds) cyclic aliphatic hydrocarbons containing one to three rings having from three to twelve carbon atoms (i.e., C₃₋₁₂ cycloalkyl) or the number of carbons designated. In one embodiment, the cycloalkyl group has two rings. In one embodiment, the cycloalkyl group has one ring. In another embodiment, the cycloalkyl group is chosen from a C₃₋₈ cycloalkyl group. In another embodiment, the cycloalkyl group is chosen from a C₃₋₆ cycloalkyl group. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, and cyclopentenyl, cyclohexenyl.

In the present disclosure, the term “optionally substituted cycloalkyl” as used by itself or as part of another group means that the cycloalkyl as defined above is either unsubstituted or substituted with one, two, or three substituents independently chosen from halo, nitro, cyano, hydroxy, amino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, and (heterocyclo)alkyl. In one embodiment, the optionally substituted cycloalkyl is substituted with two substituents. In another embodiment, the optionally substituted cycloalkyl is substituted with one substituent.

In the present disclosure, the term “cycloalkylenyl” as used herein by itself or part of another group refers to a divalent form of an optionally substituted cycloalkyl group. Non-limiting examples of a 5 cycloalkylenyl include:

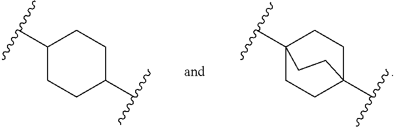 [SMILES: *C1CCC(*)CC1 ; *C12CCC(*)(CC1)CC2]

In the present disclosure, the term “alkenyl” as used by itself or as part of another group refers to an alkyl group as defined above containing one, two or three carbon-to-carbon double bonds. In one embodiment, the alkenyl group is chosen from a C₂₋₆ alkenyl group. In another embodiment, the alkenyl group is chosen from a C₂₋₄ alkenyl group. Non-limiting exemplary alkenyl groups include ethenyl, propenyl, isopropenyl, butenyl, sec-butenyl, pentenyl, and hexenyl.

In the present disclosure, the term “optionally substituted alkenyl” as used herein by itself or as part of another group means the alkenyl as defined above is either unsubstituted or substituted with one, two or three substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, aryl, heteroaryl, or heterocyclo.

In the present disclosure, the term “alkynyl” as used by itself or as part of another group refers to an alkyl group as defined above containing one to three carbon-to-carbon triple bonds. In one embodiment, the alkynyl has one carbon-to-carbon triple bond. In one embodiment, the alkynyl group is chosen from a C₂₋₆ alkynyl group. In another embodiment, the alkynyl group is chosen from a C₂₋₄ alkynyl group. Non-limiting exemplary alkynyl groups include ethynyl, propynyl, butynyl, 2-butynyl, pentynyl, and hexynyl groups.

In the present disclosure, the term “optionally substituted alkynyl” as used herein by itself or as part of another group means the alkynyl as defined above is either unsubstituted or substituted with one, two or three substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, aryl, heteroaryl, or heterocyclo.

In the present disclosure, the term “haloalkyl” as used by itself or as part of another group refers to an alkyl group substituted by one or more fluorine, chlorine, bromine and/or iodine atoms. In one embodiment, the alkyl group is substituted by one, two, or three fluorine and/or chlorine atoms. In another embodiment, the haloalkyl group is chosen from a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “hydroxyalkyl” as used by itself or as part of another group refers to an alkyl group substituted with one or more, e.g., one, two, or three, hydroxy groups. In one embodiment, the hydroxyalkyl group is a monohydroxyalkyl group, i.e., substituted with one hydroxy group. In another embodiment, the hydroxyalkyl group is a dihydroxyalkyl group, i.e., substituted with two hydroxy groups, e.g.,

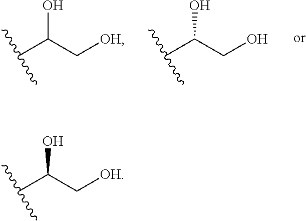 [SMILES: *C(O)CO ; *[C@H](O)CO ; "or" ; *[C@@H](O)CO]

In another embodiment, the hydroxyalkyl group is chosen from a C₁₋₄ hydroxyalkyl group. Non-limiting exemplary hydroxyalkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

In the present disclosure, the term “alkoxy” as used by itself or as part of another group refers to an optionally substituted alkyl, optionally substituted cycloalkyl, optionally substituted alkenyl or optionally substituted alkynyl attached to a terminal oxygen atom. In one embodiment, the alkoxy group is chosen from a C₁₋₄ alkoxy group. In another embodiment, the alkoxy group is chosen from a C₁₋₄ alkyl attached to a terminal oxygen atom, e.g., methoxy, ethoxy, and tert-butoxy.

In the present disclosure, the term “alkylthio” as used by itself or as part of another group refers to a sulfur atom substituted by an optionally substituted alkyl group. In one embodiment, the alkylthio group is chosen from a C₁₋₄ alkylthio group. Non-limiting exemplary alkylthio groups include —SCH₃, and —SCH₂CH₃.

In the present disclosure, the term “alkoxyalkyl” as used by itself or as part of another group refers to an alkyl group substituted with an alkoxy group. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

In the present disclosure, the term “haloalkoxy” as used by itself or as part of another group refers to a haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryl” as used by itself or as part of another group refers to a monocyclic or bicyclic aromatic ring system having from six to fourteen carbon atoms (i.e., C₆-C₁₄ aryl). Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is chosen from phenyl or naphthyl.

In the present disclosure, the term “optionally substituted aryl” as used herein by itself or as part of another group means that the aryl as defined above is either unsubstituted or substituted with one to five substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl.

In one embodiment, the optionally substituted aryl is an optionally substituted phenyl. In one embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, and 3-chloro-4-fluorophenyl. The term optionally substituted aryl is meant to include groups having fused optionally substituted cycloalkyl and fused optionally substituted heterocyclo rings. Non-limiting examples include:

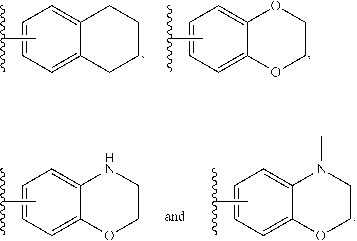

In the present disclosure, the term “phenylenyl” as used herein by itself or part of another group refers to a divalent form of an optionally substituted phenyl group. Non-limiting examples include:

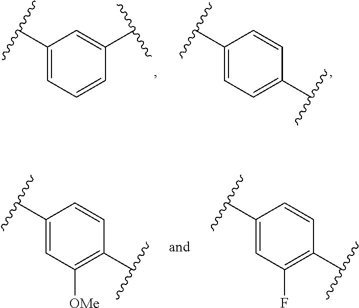 [SMILES: *c1cccc(*)c1 ; *c1ccc(*)cc1 ; *c1ccc(*)c(OC)c1 ; *c1ccc(*)c(F)c1]

In the present disclosure, the term “aryloxy” as used by itself or as part of another group refers to an optionally substituted aryl attached to a terminal oxygen atom. A non-limiting exemplary aryloxy group is PhO—.

In the present disclosure, the term “aralkyloxy” as used by itself or as part of another group refers to an aralkyl group attached to a terminal oxygen atom. A non-limiting exemplary aralkyloxy group is PhCH₂O—.

In the present disclosure, the term “heteroaryl” or “heteroaromatic” refers to monocyclic and bicyclic aromatic ring systems having 5 to 14 ring atoms (i.e., C₅-C₁₄ heteroaryl), wherein at least one carbon atom of one of the rings is replaced with a heteroatom independently selected from the group consisting of oxygen, nitrogen and sulfur. In one embodiment, the heteroaryl contains 1, 2, 3, or 4 heteroatoms independently selected from the group consisting of oxygen, nitrogen and sulfur. In one embodiment, the heteroaryl has three heteroatoms. In another embodiment, the heteroaryl has two heteroatoms. In another embodiment, the heteroaryl has one heteroatom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl), isoxazolyl (e.g., isoxazol-3-yl, isoxazol-4-yl, and isoxazol-5-yl), and indazolyl (e.g., 1H-indazol-3-yl). The term “heteroaryl” is also meant to include possible N-oxides. A non-limiting exemplary N-oxide is pyridyl N-oxide.

In one embodiment, the heteroaryl is a 5- or 6-membered heteroaryl. In one embodiment, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Non-limiting exemplary 5-membered heteroaryl groups include thienyl, furyl, pyrrolyl, oxazolyl, pyrazolyl, imidazolyl, thiazolyl, isothiazolyl, and isoxazolyl.

In another embodiment, the heteroaryl is a 6-membered heteroaryl, e.g., the heteroaryl is a monocyclic aromatic ring system having 6 ring atoms wherein at least one carbon atom of the ring is replaced with a nitrogen atom. Non-limiting exemplary 6-membered heteroaryl groups include pyridyl, pyrazinyl, pyrimidinyl, and pyridazinyl.

In the present disclosure, the term “optionally substituted heteroaryl” as used by itself or as part of another group means that the heteroaryl as defined above is either unsubstituted or substituted with one to four substituents, e.g., one or two substituents, independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. In one embodiment, the optionally substituted heteroaryl has one substituent. Any available carbon or nitrogen atom can be substituted. Non-limiting exemplary optionally substituted 5-membered heteroaryl groups include, but are not limited to

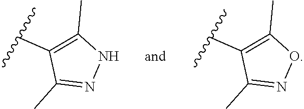

The term optionally substituted heteroaryl is also meant to include groups having fused optionally substituted cycloalkyl and fused optionally substituted heterocyclo rings. Non-limiting examples include:

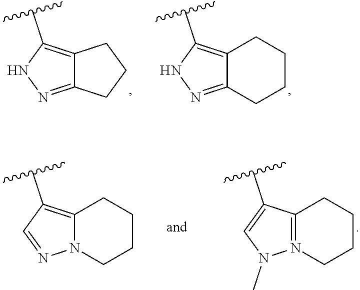

In the present disclosure, the term “heteroarylenyl” as used herein by itself or part of another group refers to a divalent form of an optionally substituted heteroaryl group. In one embodiment, the heteroarylenyl is a 5-membered heteroarylenyl. Non-limiting examples of a 5-membered heteroarylenyl include:

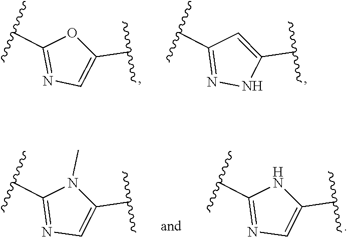

In one embodiment, the heteroarylenyl is a 6-membered heteroarylenyl. Non-limiting examples of a 6-membered heteroarylenyl include:

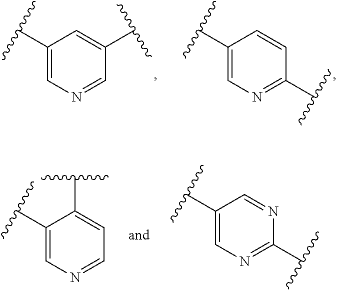 [SMILES: *c1cncc(*)c1 ; *c1ccc(*)nc1 ; *c1ccncc1* ; *c1cnc(*)nc1]

In the present disclosure, the term “heterocycle” or “heterocyclo” as used by itself or as part of another group refers to saturated and partially unsaturated (e.g., containing one or two double bonds) cyclic groups containing one, two, or three rings having from three to fourteen ring members (i.e., a 3- to 14-membered heterocyclo) wherein at least one carbon atom of one of the rings is replaced with a heteroatom. Each heteroatom is independently selected from the group consisting of oxygen, sulfur, including sulfoxide and sulfone, and/or nitrogen atoms, which can be oxidized or quaternized. The term “heterocyclo” is meant to include groups wherein a ring —CH₂— is replaced with a —C(═O)—, for example, cyclic ureido groups such as 2-imidazolidinone and cyclic amide groups such as β-lactam, γ-lactam, δ-lactam, ε-lactam, and piperazin-2-one. The term “heterocyclo” is also meant to include groups having fused optionally substituted aryl groups, e.g., indolinyl, chroman-4-yl. In one embodiment, the heterocyclo group is chosen from a 5- or 6-membered cyclic group containing one ring and one or two oxygen and/or nitrogen atoms. The heterocyclo can be optionally linked to the rest of the molecule through any available carbon or nitrogen atom. Non-limiting exemplary heterocyclo groups include dioxanyl, tetrahydropyranyl, 2-oxopyrrolidin-3-yl, piperazin-2-one, piperazine-2,6-dione, 2-imidazolidinone, piperidinyl, morpholinyl, piperazinyl, pyrrolidinyl, and indolinyl.

In the present disclosure, the term “optionally substituted heterocyclo” as used herein by itself or part of another group means the heterocyclo as defined above is either unsubstituted or substituted with one to four substituents independently selected from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, alkoxycarbonyl, CF₃C(═O)—, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. Substitution may occur on any available carbon or nitrogen atom, or both. Non-limiting exemplary optionally substituted heterocyclo groups include:

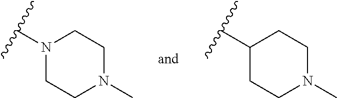

In the present disclosure, the term “amino” as used by itself or as part of another group refers to —NR^{10a}R^{10b}, wherein R^{10a} and R^{10b} are each independently hydrogen, alkyl, hydroxyalkyl, optionally substituted cycloalkyl, optionally substituted aryl, optionally substituted heterocyclo, or optionally substituted heteroaryl, or R^{10a} and R^{10b} are taken together to form a 3- to 8-membered optionally substituted heterocyclo. Non-limiting exemplary amino groups include —NH₂ and —N(H)(CH₃).

In the present disclosure, the term “(amino)alkyl” as used by itself or as part of another group refers to an alkyl group substituted with an amino group. Non-limiting exemplary amino alkyl groups include —CH₂CH₂NH₂, and —CH₂CH₂N(H)CH₃, —CH₂CH₂N(CH₃)₂, and —CH₂N(H)cyclopropyl.

In the present disclosure, the term “carboxamido” as used by itself or as part of another group refers to a radical of formula —C(═O)NR^{9a}R^{9b}, wherein R^{9a} and R^{9b} are each independently hydrogen, optionally substituted alkyl, hydroxyalkyl, optionally substituted cycloalkyl, optionally substituted aryl, optionally substituted heterocyclo, or optionally substituted heteroaryl, or R^{9a} and R^{9b} taken together with the nitrogen to which they are attached form a 3- to 8-membered optionally substituted heterocyclo group. In one embodiment, R^{9a} and R^{9b} are each independently hydrogen or optionally substituted alkyl. In one embodiment, R^{9a} and R^{9b} are taken together to taken together with the nitrogen to which they are attached form a 3- to 8-membered optionally substituted heterocyclo group. Non-limiting exemplary carboxamido groups include, but are not limited to, —CONH₂, —CON(H)CH₃, —CON(CH₃)₂, —CON(H)Ph,

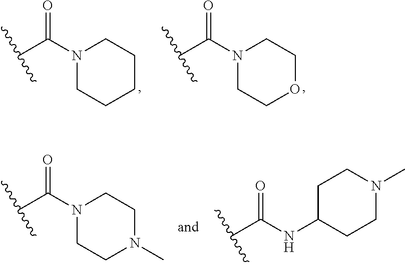

In the present disclosure, the term “sulfonamido” as use by itself or as part of another group refers to a radical of the formula —SO₂NR^{8a}R^{8b}, wherein R^{8a} and R^{8b} are each independently hydrogen, optionally substituted alkyl, or optionally substituted aryl, or R^{8a} and R^{8b} taken together with the nitrogen to which they are attached from a 3- to 8-membered heterocyclo group. Non-limiting exemplary sulfonamido groups include —SO₂NH₂, —SO₂N(H)CH₃, and —SO₂N(H)Ph.

In the present disclosure, the term “alkylcarbonyl” as used by itself or as part of another group refers to a carbonyl group, i.e., —C(═O)—, substituted by an alkyl group. A non-limiting exemplary alkylcarbonyl group is —COCH₃.

In the present disclosure, the term “arylcarbonyl” as used by itself or as part of another group refers to a carbonyl group, i.e., —C(═O)—, substituted by an optionally substituted aryl group. A non-limiting exemplary arylcarbonyl group is —COPh.

In the present disclosure, the term “alkoxycarbonyl” as used by itself or as part of another group refers to a carbonyl group, i.e., —C(═O)—, substituted by an alkoxy group. Non-limiting exemplary alkoxycarbonyl groups include —C(═O)OMe, —C(═O)OEt, and —C(═O)OtBu.

In the present disclosure, the term “alkylsulfonyl” as used by itself or as part of another group refers to a sulfonyl group, i.e., —SO₂—, substituted by any of the above-mentioned optionally substituted alkyl groups. A non-limiting exemplary alkylsulfonyl group is —SO₂CH₃.

In the present disclosure, the term “arylsulfonyl” as used by itself or as part of another group refers to a sulfonyl group, i.e., —SO₂—, substituted by any of the above-mentioned optionally substituted aryl groups. A non-limiting exemplary arylsulfonyl group is —SO₂Ph.

In the present disclosure, the term “mercaptoalkyl” as used by itself or as part of another group refers to any of the above-mentioned alkyl groups substituted by a —SH group.

In the present disclosure, the term “carboxy” as used by itself or as part of another group refers to a radical of the formula —COOH.

In the present disclosure, the term “carboxyalkyl” as used by itself or as part of another group refers to any of the above-mentioned alkyl groups substituted with a —COOH. A non-limiting exemplary carboxyalkyl group is —CH₂CO₂H.

In the present disclosure, the terms “aralkyl” or “arylalkyl” as used by themselves or as part of another group refers to an alkyl group substituted with one, two, or three optionally substituted aryl groups. In one embodiment, the optionally substituted aralkyl group is a C₁₋₄ alkyl substituted with one optionally substituted aryl group. In one embodiment, the optionally substituted aralkyl group is a C₁ or C₂ alkyl substituted with one optionally substituted aryl group. In one embodiment, the optionally substituted aralkyl group is a C₁ or C₂ alkyl substituted with one optionally substituted phenyl group. Non-limiting exemplary optionally substituted aralkyl groups include benzyl, phenethyl, —CHPh₂, —CH₂(4-F-Ph), —CH₂(4-Me-Ph), —CH₂(4-CF₃-Ph), and —CH(4-F-Ph)₂.

In the present disclosure, the terms “(heterocyclo)alkyl” as used by itself or part of another group refers to an alkyl group substituted with an optionally substituted heterocyclo group. In one embodiment, the (heterocyclo)alkyl is a C₁₋₄ alkyl substituted with one optionally substituted heterocyclo group. Non-limiting exemplary (heterocyclo)alkyl groups include:

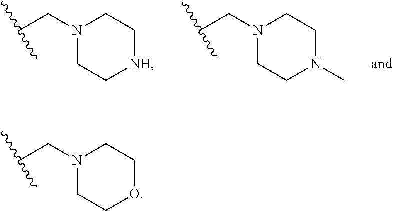

The present disclosure encompasses any of the Compounds of the Disclosure being isotopically-labelled, i.e., radiolabeled, by having one or more atoms replaced by an atom having a different atomic mass or mass number. Examples of isotopes that can be incorporated into Compounds of the Disclosure include isotopes of hydrogen, carbon, nitrogen, sulfur, oxygen, fluorine, and chlorine, such as ²H (or deuterium (D)), ³H, ¹¹C, ¹³C, ¹⁴C, ¹⁵N, ¹⁸O, ¹⁷O, ³⁵S, ¹⁸F, and ³⁶Cl, e.g., ²H, ³H, and ¹³C. In one embodiment, a portion of the atoms at a position within a Compound of the Disclosure are replaced, i.e., the Compound of the Disclosure is enriched at a position with an atom having a different atomic mass or mass number. In one embodiment, at least about 1% of the atoms are replaced with an atom having a different atomic mass or mass number. In another embodiment, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the atoms are replaced with an atom having a different atomic mass or mass number. Isotopically-labeled Compounds of the Disclosure can be prepared by methods known in the art.

### EXAMPLES

### Example 1

### General Synthesis

The synthesis of VHL ligands (49a, 49b) is outlined in Scheme 1. Briefly, (4-bromophenyl)methanamine (42a) was protected by Boc₂O to generate 43a. An intermediate (44) was obtained through the Heck reaction of 43a and 4-methylthiazole, and subsequent deprotection of the Boc group gave compound 45a. Amide coupling of 46 with 47 followed by hydrolysis of the methyl ester produced the key intermediate (48). Amidation of 48 with 45a in the presence of 1-[bis(dimethylamino)methylene]-1H-1,2,3-triazolo[4,5-b]-pyridinium 3-oxide hexafluorophosphate (HATU) and N,N-diisopropylethylamine (DIPEA) at rt in dimethyl-formamide (DMF) gave the target compound (49a)

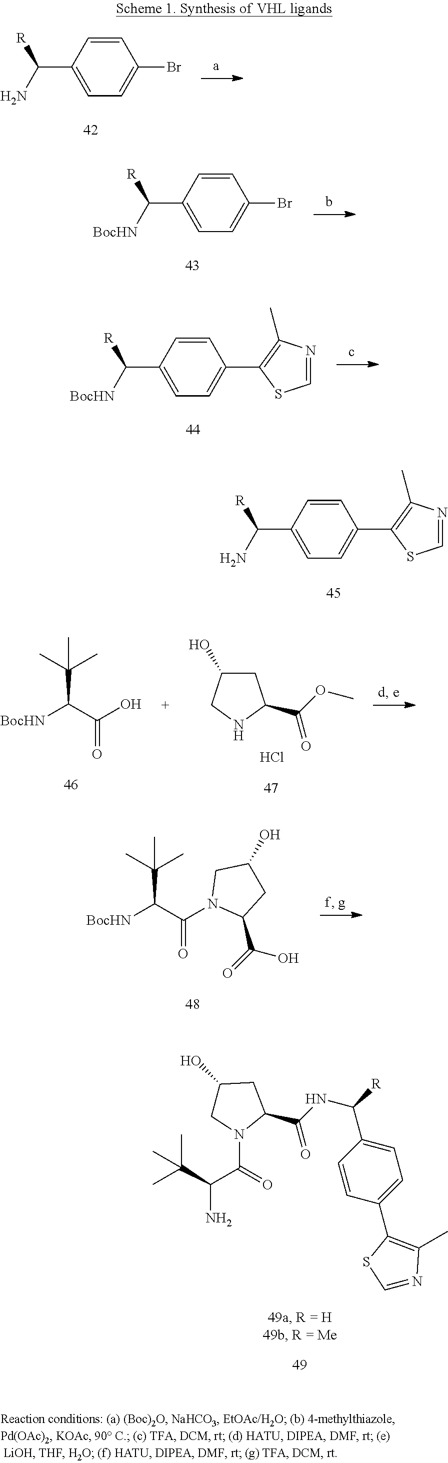

### Example 2

### Synthesis of AR Degraders

As shown in Scheme 2, compounds 8-17 were synthesized by the amidation of intermediate 52 and the VHL ligand (49a). Intermediate 52 was produced by amidation of compound 50 and a series of amines. Compound 50 was synthesized by the hydrolysis of commercial enzalutamide (4). Compound 18 was synthesized from the amidation reaction of compound 53 which was prepared from intermediate 50. The synthesis of compound 26 was through several amidation reactions starting from intermediate 50.

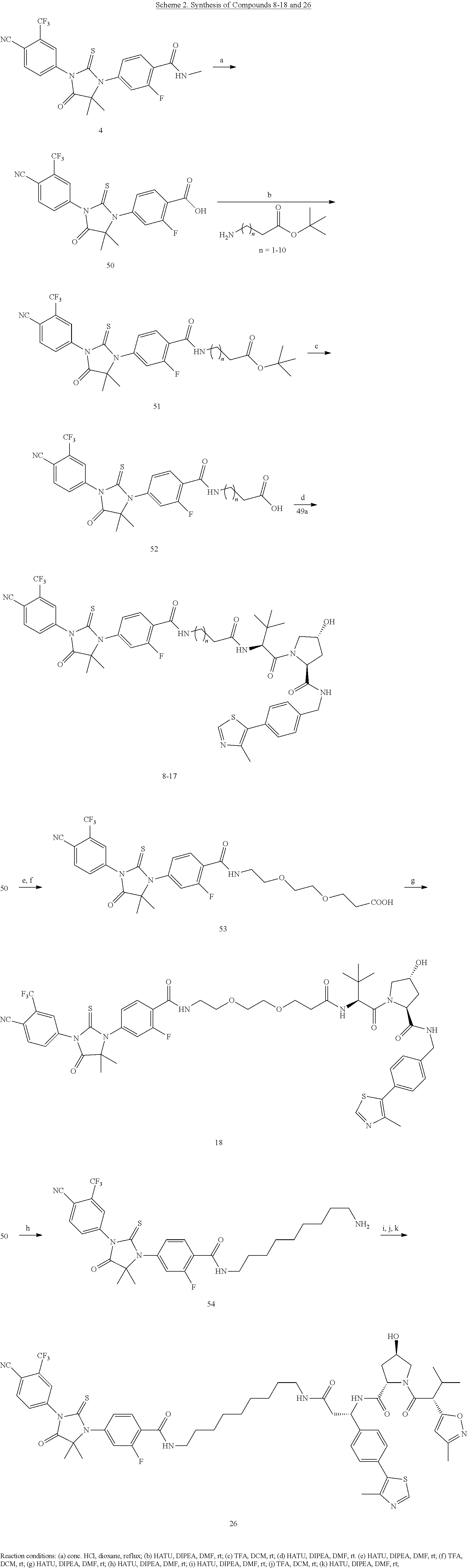

The synthesis of compound 19 is shown in Scheme 3. First, the key intermediate (57) was synthesized by coupling of compounds 55 and 56. Then Sonogashira coupling reaction of 57 with 1-bromo-4-ethynylbenzene in the presence of CuI and PdCl₂(PPh₃)₂ at 100° C. in DMF/TEA gave compound 58. The intermediate (59) was obtained from 58 in the presence of 2-hydroxy-2-methylpropanenitrile. The target compound (19) can be obtained through the amidation reaction of VHL ligand (49a) with 60 which in turn was derived from intermediate 59.

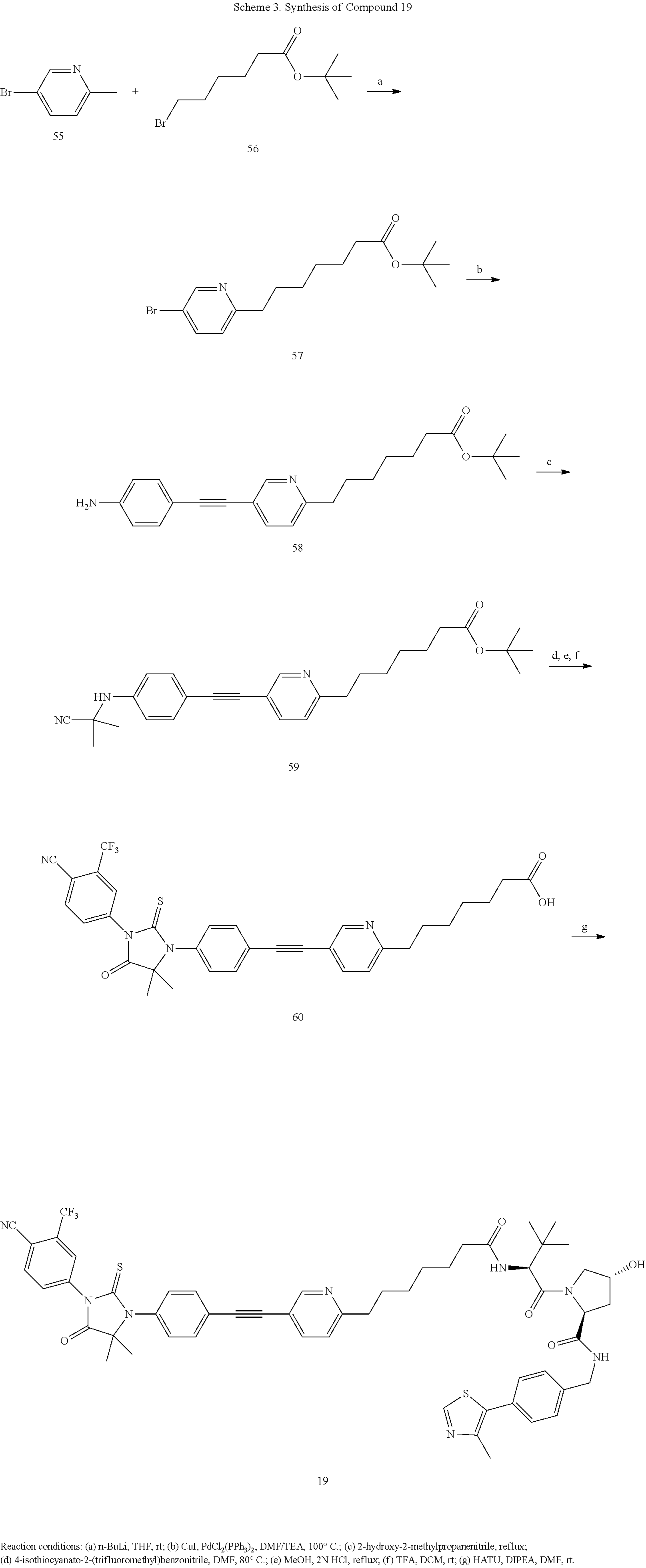

As shown in Scheme 4, compounds 20-25, 27 and 28 were synthesized according to the following procedure. A Heck reaction of compounds 61 and 62 gave the key intermediate (63). The other key intermediate (68) was made through 4 steps from the starting material (64) according to a published method.³⁷ Then a Sonogashira coupling reaction of 69 and 70 in the presence of dichlorobispalladium (PdCl₂(PPh₃)₂), cuprous iodide (CuI) and triethylamine (TEA) in dry DMF gave the key linker portion (71). A mixture of 71 and acetone cyanohydrin was heated to 80° C. and stirred for 4 h. The medium was concentrated and dried under vacuum to yield 72 which was used in the next step without further purification. A mixture of 4-isothiocyanato-2-(trifluoromethyl)benzonitrile and 72 in DMF was stirred overnight. Then, MeOH and 2N HCl were added to this mixture to give the cyclized intermediate (73). A substitution reaction between 73 and tert-butyl 2-bromoacetate in the presence of K₂CO₃ and KI gave the key intermediate (74). Amidation of 74 and the VHL ligand (49) gave the target compounds (20-25). Compounds 27 and 28 were obtained through 2 amidation reactions.

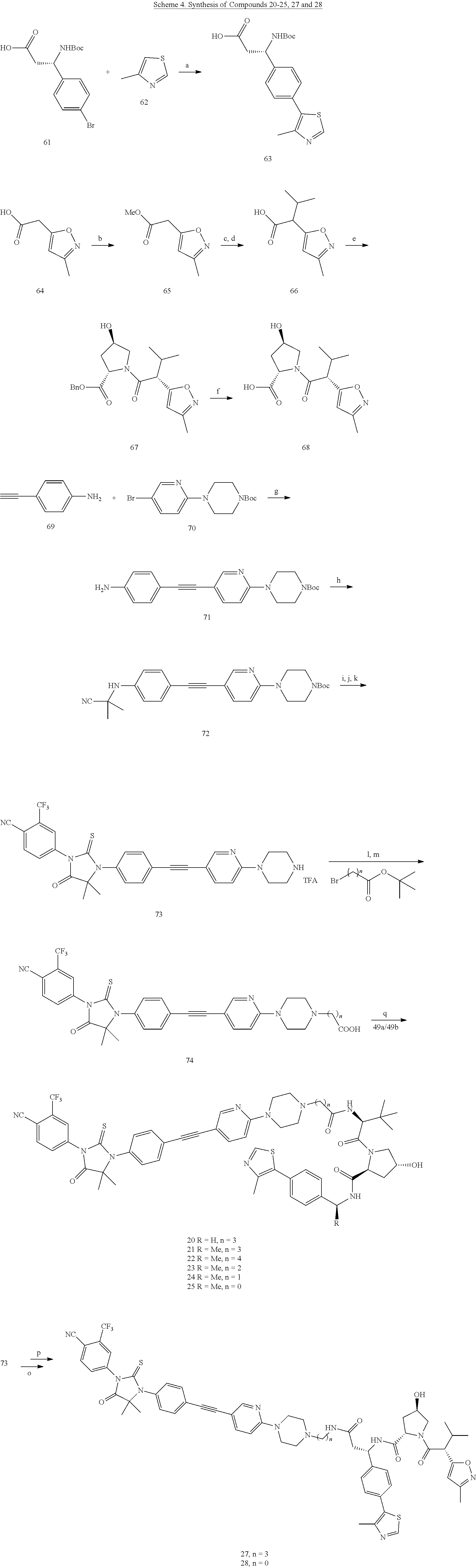

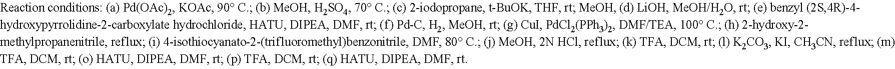

The synthesis of compounds 29-30 is shown in Scheme 5. Sonogashira coupling reaction of 75 and 76 in the presence of dichlorobispalladium (PdCl₂(PPh₃)₂), cuprous iodide (CuI) and TEA in dry DMF gave the key linker portion (77). A mixture of 77 and acetone cyanohydrin was heated to 80° C. and stirred for 4 h. The medium was concentrated and dried under vacuum to give 78 which was used in the next step without further purification. A mixture of 4-isothiocyanato-2-(trifluoromethyl)benzonitrile and 78 in DMF was stirred overnight at rt. Then MeOH and 2N HCl were added to this mixture to give the cyclized intermediate (79). A substitution reaction of 79 with tert-butyl 4-bromobutanoate in the presence of K₂CO₃ and KI gave the key intermediate (80). Amidation of 80 and 63 gave the compound 81. In the last step, the compounds 29-30 were obtained through amidation reaction of the intermediate 81 with 68.

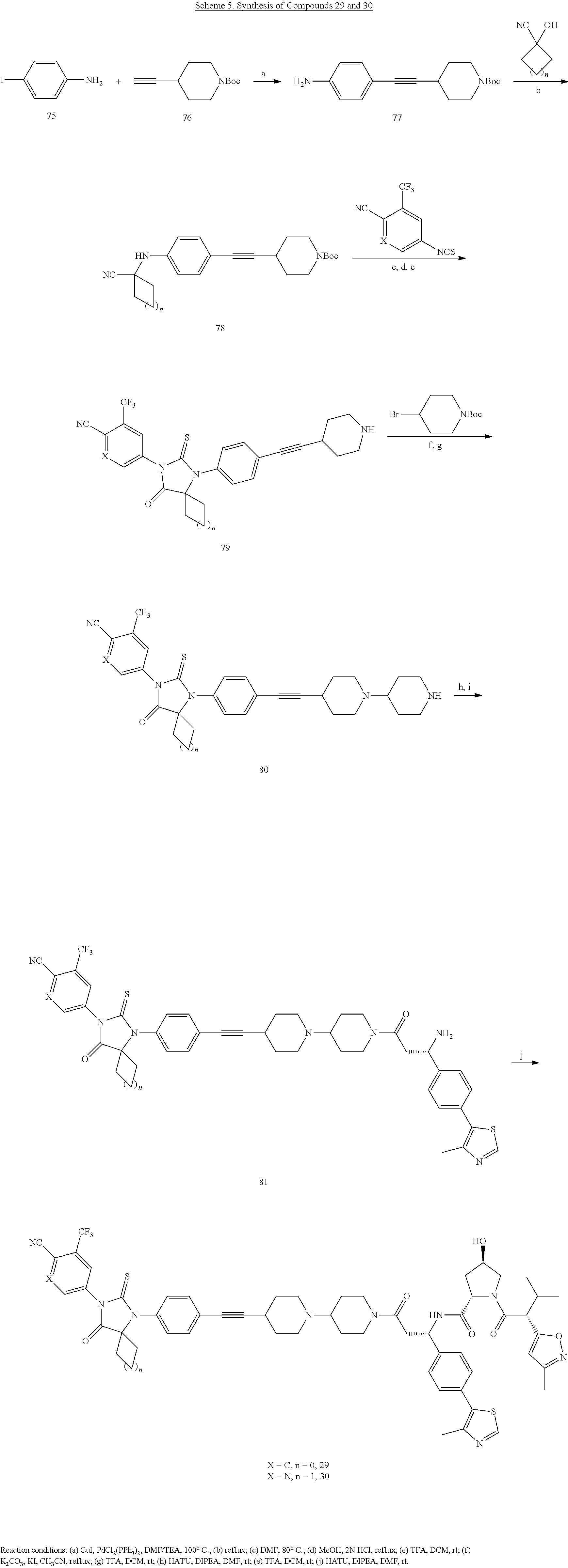

The synthesis of compounds 32 and 34-36 is shown in Scheme 6. The compound 83 was synthesized from the Sonogashira coupling reaction of the starting material (82) and 76. Compound 84 was made through the substitution reaction of intermediate 83 and tert-butyl 4-bromopiperidine-1-carboxylate. As shown in Scheme 6, the key intermediate (88) was synthesized in two steps. Compound 89 can be obtained from the amidation reaction of compound 88 with different amino acids. Finally, the target compounds were obtained through the amidation of intermediate 89 and various VHL fragments.

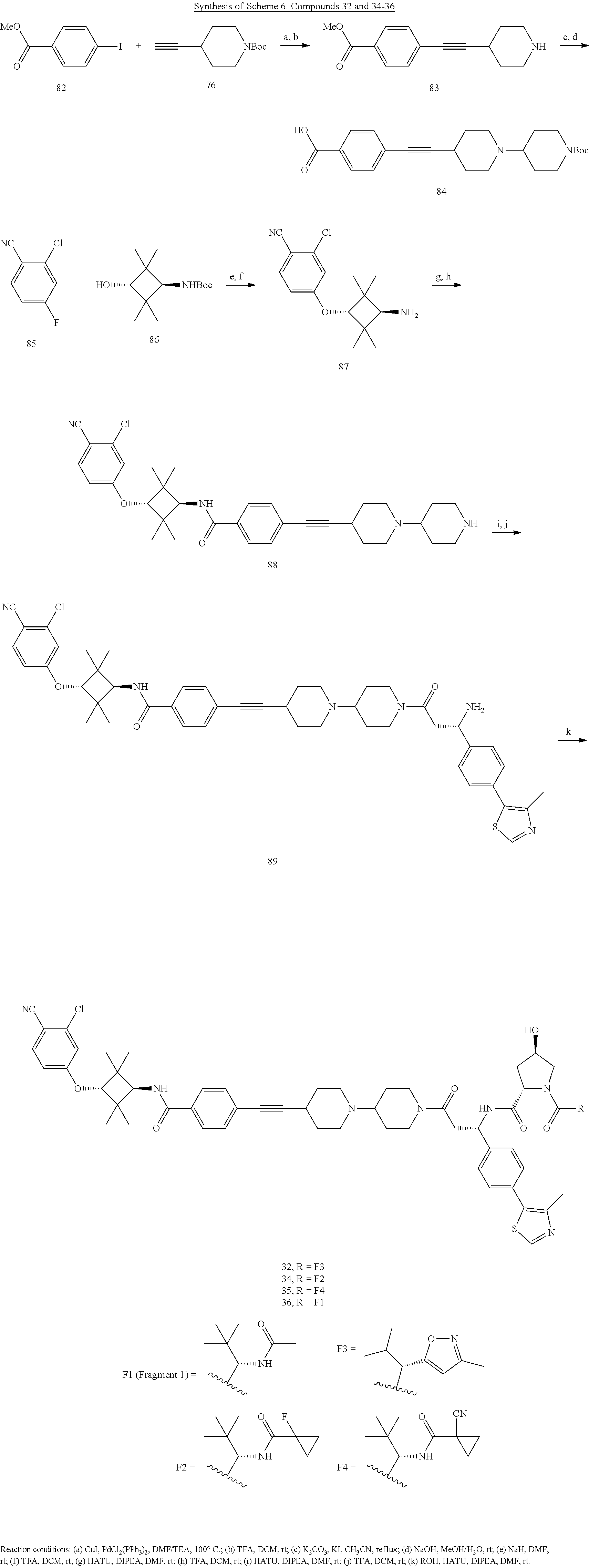

Compound 31 was synthesized according to the method shown in Scheme 7. A substitution reaction of compounds 90 and 91 gave the key intermediate (92). The key AR antagonist portion (96) can be obtained from the reaction of compounds 93 and 94 followed by a further oxidation reaction. A Sonogashira coupling reaction of compounds 96 and 92 gave the compound 97, and the target compound 31 was obtained by two amidation reactions.

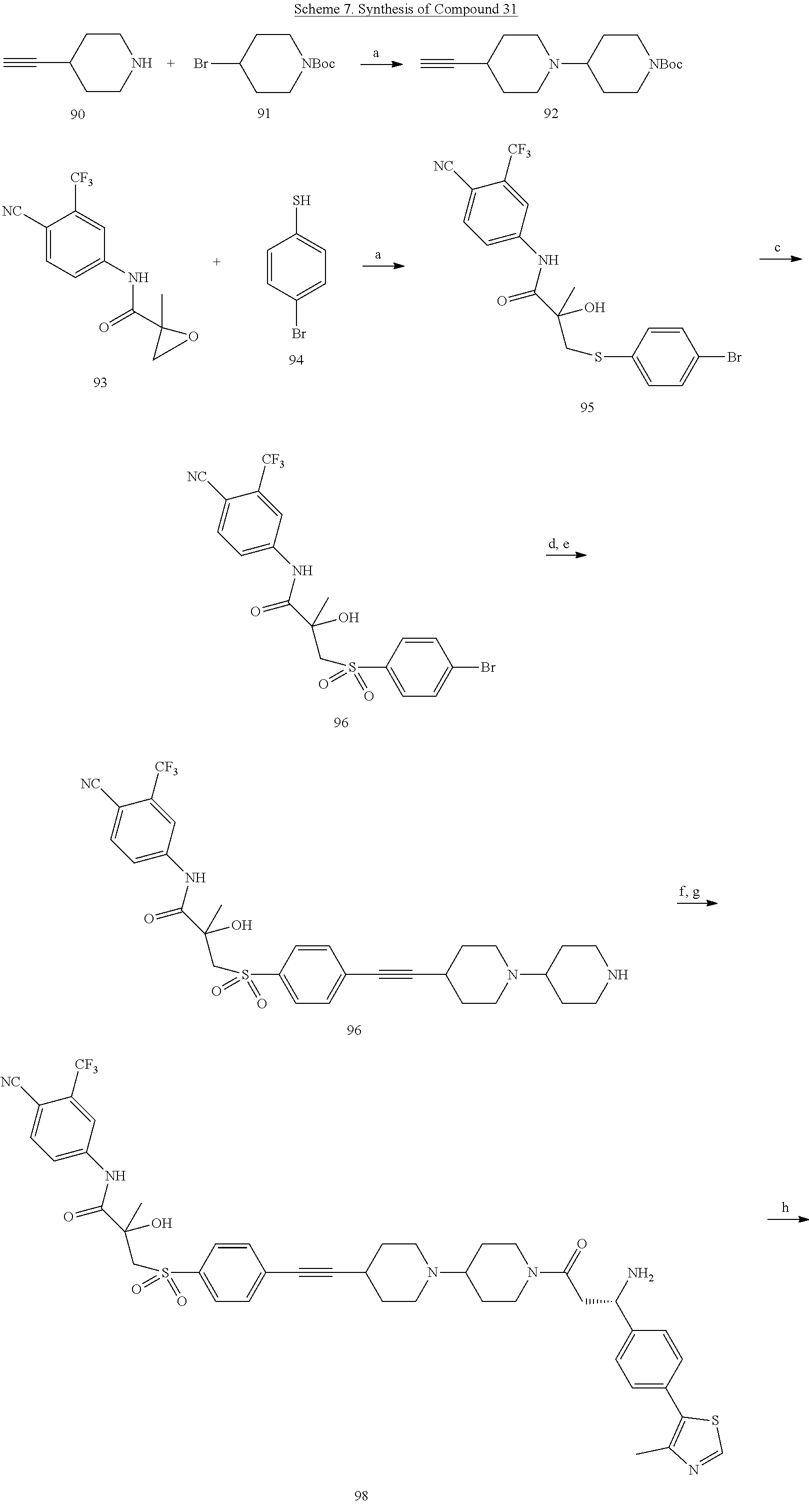

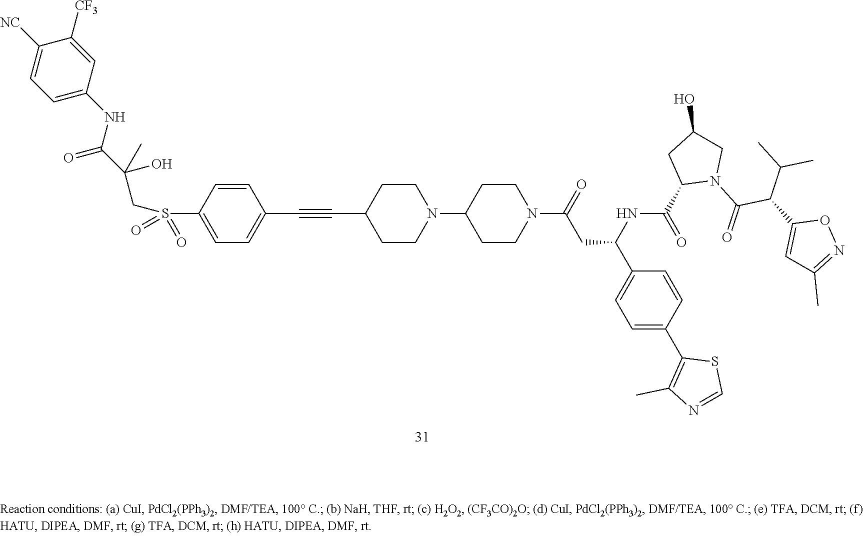 [SMILES: Cc1cc([C@H](C(=O)N2C[C@H](O)C[C@H]2C(=O)N[C@@H](CC(=O)N2CCC(N3CCC(C#Cc4ccc(S(=O)(=O)CC(C)(O)C(=O)Nc5ccc(C#N)c(C(F)(F)F)c5)cc4)CC3)CC2)c2ccc(-c3scnc3C)cc2)C(C)C)on1]

The synthesis of compound 33 is shown in Scheme 8. Compound 101 was synthesized by the Michael addition reaction of compound 99 with 100, and compound 104 was made by the reaction of compound 101 with NH₂NH₂. The reaction of compound 102 with compound 85 gave the AR antagonist (103). Sonogashira coupling reaction of 103 with 92 in the presence of CuI and PdCl₂(PPh₃)₂ at rt in DMF/TEA gave compound 104. Finally, the target compound 33 was obtained from an amidation reaction.

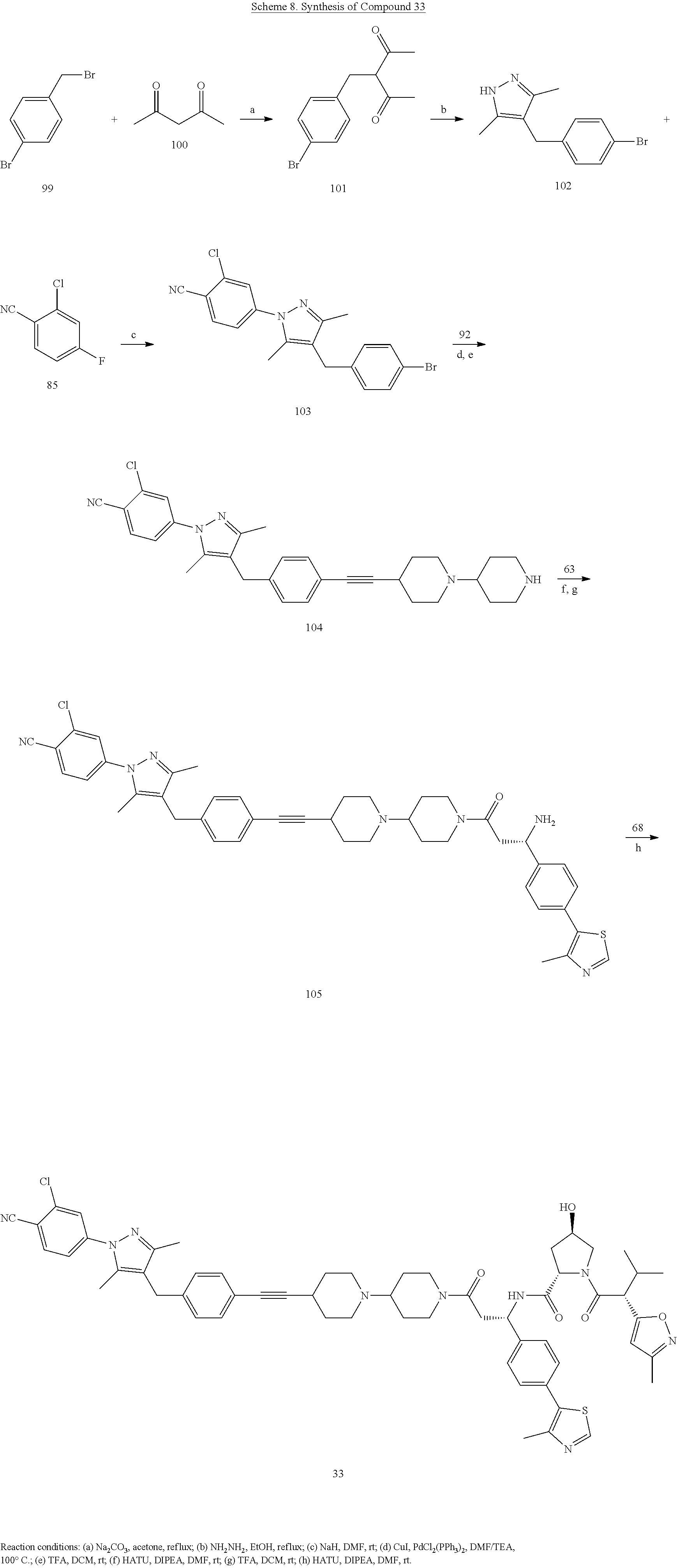

As shown in Scheme 9, compounds 39-41 were made according the following procedure. In detail, compound 107 was synthesized by the amidation reaction of compounds 87 and 106. Compound 108 was made by hydrolysis reaction of intermediate 107, and intermediate 109 was synthesized by the amidation of compound 108 with nonane-1,9-diamine. Finally, the target compound (39) was synthesized by two amidation reactions. Compound 41 was made through the substitution reaction of 88 with 111, and compound 40 was synthesized by the substitution reaction of 111 and 112, which was produced by amidation reaction of decane-1,10-diamine with compound 108.

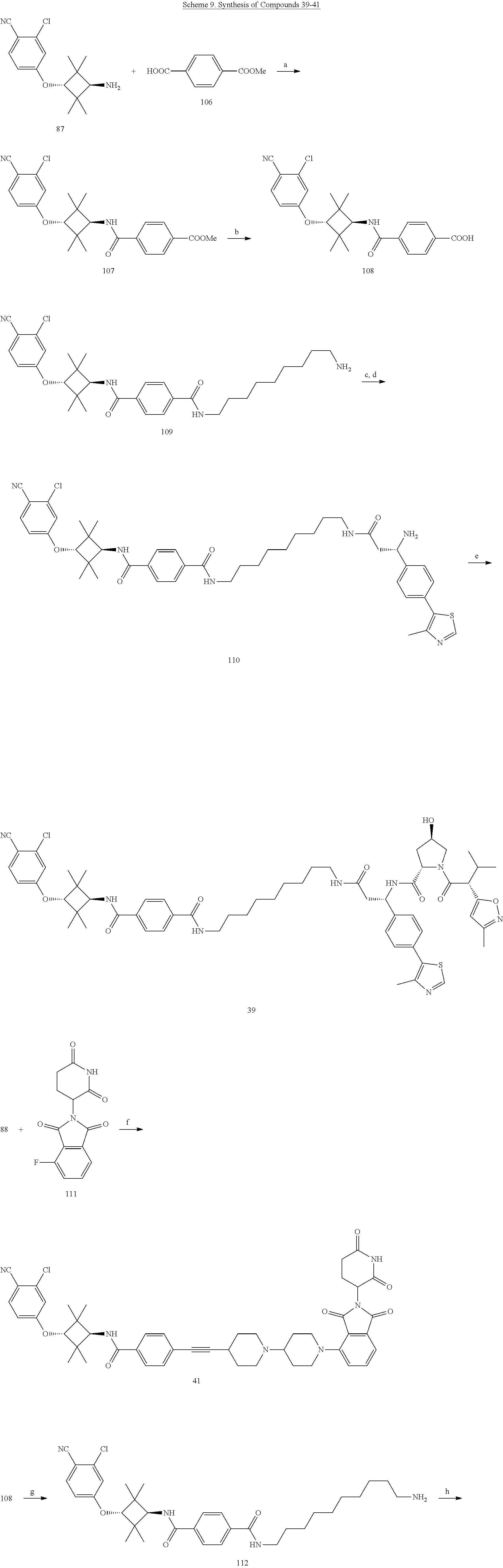

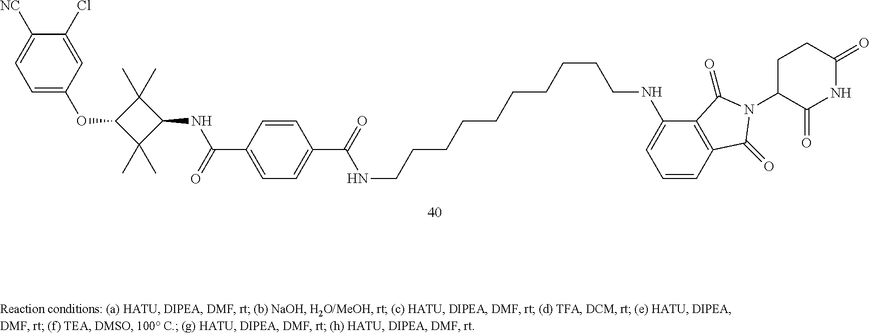 [SMILES: CC1(C)[C@H](NC(=O)c2ccc(C(=O)NCCCCCCCCCCNc3cccc4c3C(=O)N(C3CCC(=O)NC3=O)C4=O)cc2)C(C)(C)[C@H]1Oc1ccc(C#N)c(Cl)c1]

In Scheme 10, compounds 37 and 38 were synthesized according to previously published methods as shown in Scheme 4

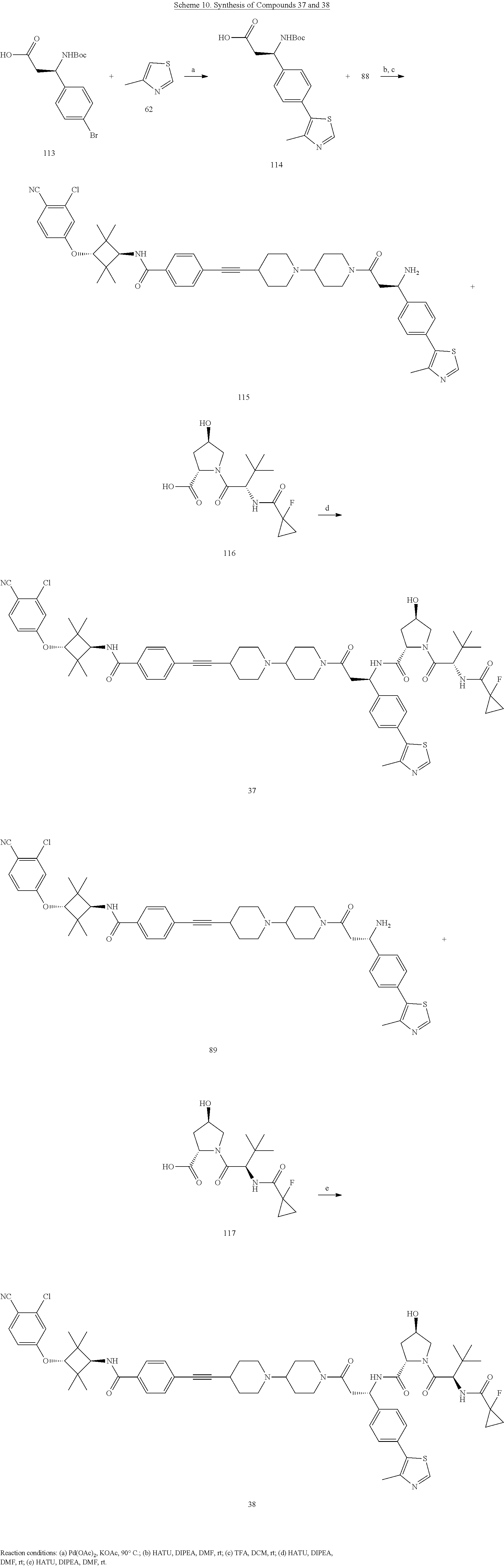

### Example 3

### Synthetic Procedures

Chemistry. General Experiment and Information. Unless otherwise noted, all purchased reagents were used as received without further purification. ¹H NMR and ¹³C NMR spectra were recorded on a Bruker Advance 400 MHz spectrometer. ¹H NMR spectra were reported in parts per million (ppm) downfield from tetramethylsilane (TMS). All ¹³C NMR spectra were reported in ppm and obtained with ¹H decoupling. In reported spectral data, the format (8) chemical shift (multiplicity, J values in Hz, integration) was used with the following abbreviations: s=singlet, d=doublet, t=triplet, q=quartet, m=multiplet. Mass spectral (MS) analysis was carried out with a Waters UPLC mass spectrometer. The final compounds were all purified by C18 reverse phase preparative HPLC column with solvent A (0.1% TFA in H₂O) and solvent B (0.1% TFA in CH₃CN) as eluents. The purity of all the final compounds was confirmed to be >95% by UPLC-MS or UPLC.

### General Procedure for Synthesis of Compounds 8-17

Conc. HCl (5 mL) was added to a solution of enzalutamide (4) (4.64 g, 10 mmol) in dioxane (50 mL). The reaction mixture was refluxed for 2 h. The reaction mixture was quenched with H₂O and extracted with DCM. The organic layer was separated, washed with brine, dried, and evaporated. The final compound 50 was obtained by flash column chromatography (hexane/EtOAc=4:1) with 95% yield.

DIPEA (5 eq.) and HATU (1.2 eq.) were added to a solution of compound 50 (451 mg, 1 mmol) and a series of linear amines (1.1 eq.) in DMF (2 mL). After 30 min at rt, the mixture was subject to HPLC purification to afford compound 51 with 80-90% yields.

A solution of compound 51 in 1:1 TFA/DCM was stirred at rt for 30 min. The solvents were evaporated under reduced pressure to give the corresponding deprotected intermediate 52 (TFA salt) that was used in the following reactions without further purification (95% yield).

DIPEA (5 eq.) and HATU (1.2 eq.) were added to a solution of compound 52 (52.2 mg, 0.1 mmol) and compound 49a (1.1 eq.) in DMF (2 mL). After 30 min at rt, the mixture was subject to HPLC purification to afford compound 8 in 90% yield. Following the procedures used to prepare compound 8, compounds 9-17 with different chain lengths were obtained by the same methods.

(4R)-1-((S)-2-(3-(4-(3-(4-Cyano-3-(trifluoromethyl)phenyl)-5,5-dimethyl-4-oxo-2-thioxoimidazolidin-1-yl)-2-fluorobenzamido)propanamido)-3,3-dimethylbutanoyl)-4-hydroxy-N-(4-(4-methylthiazol-5-yl)benzyl)pyrrolidine-2-carboxamide (8). ¹H NMR (400 MHz, MeOD-d₄) δ=8.94 (s, 1H), 8.16 (d, J=7.2 Hz, 2H), 7.99 (d, J=8.4 Hz, 1H), 7.52-7.31 (m, 6H), 4.63 (s, 1H), 4.59-4.45 (m, 3H), 4.36 (d, J=15.2 Hz, 1H), 3.90 (d, J=10.0 Hz, 1H), 3.83-3.75 (m, 1H), 3.40 (t, J=7.0 Hz, 2H), 2.49 (s, 3H), 2.44-2.00 (m, 4H), 1.60 (s, 6H), 1.04 (s, 9H), UPLC-MS calculated for C₄₅H₄₇F₄N₈O₆S₂ [M+H]⁺: 935.30, found: 935.52. UPLC-retention time: 4.77 min.

(4R)-1-((S)-2-(4-(4-(3-(4-Cyano-3-(trifluoromethyl)phenyl)-5,5-dimethyl-4-oxo-2-thioxoimidazolidin-1-yl)-2-fluorobenzamido)butanamido)-3,3-dimethylbutanoyl)-4-hydroxy-N-(4-(4-methylthiazol-5-yl)benzyl)pyrrolidine-2-carboxamide (9). ¹H NMR (400 MHz, MeOD-d₄) δ=8.93 (s, 1H), 8.16 (d, J=7.6 Hz, 2H), 7.99 (d, J=8.0 Hz, 1H), 7.50-7.32 (m, 6H), 4.63 (s, 1H), 4.61-4.45 (m, 3H), 4.35 (d, J=15.6 Hz, 1H), 3.91 (d, J=10.0 Hz, 1H), 3.83-3.55 (m, 3H), 3.41 (t, J=7.2 Hz, 2H), 2.48 (s, 3H), 2.33-2.15 (m, 3H), 2.12-2.00 (m, 3H), 1.60 (s, 6H), 1.04 (s, 9H), UPLC-MS calculated for C₄₆H₄₉F₄N₈O₆S₂ [M+H]⁺: 949.32, found: 949.58 (H⁺), UPLC-retention time: 4.92 min.

(4R)-1-((S)-2-(5-(4-(3-(4-Cyano-3-(trifluoromethyl)phenyl)-5,5-dimethyl-4-oxo-2-thioxoimidazolidin-1-yl)-2-fluorobenzamido)pentanamido)-3,3-dimethylbutanoyl)-4-hydroxy-N-(4-(4-methylthiazol-5-yl)benzyl)pyrrolidine-2-carboxamide (10). ¹H NMR (400 MHz, MeOD-d₄) δ=8.97 (s, 1H), 8.16 (d, J=7.4 Hz, 2H), 7.99 (d, J=8.4 Hz, 1H), 7.84 (dd, J=8.0 Hz, J=7.6 Hz, 1H), 7.53-7.39 (m, 4H), 7.38-7.37 (m, 2H), 4.63 (s, 1H), 4.60-4.46 (m, 3H), 4.36 (d, J=15.2 Hz, 1H), 3.90 (d, J=10.8 Hz, 1H), 3.83-3.75 (m, 1H), 3.40 (t, J=7.0 Hz, 2H), 2.51 (s, 3H), 2.38-2.15 (m, 3H), 2.13-2.02 (m, 1H), 1.69-1.55 (m, 10H), 1.04 (s, 9H), UPLC-MS calculated for C₄₇H₅₁F₄N₈O₆S₂ [M+H]⁺: 963.33, found: 963.54. UPLC-retention time: 5.15 min.

(4R)-1-((S)-2-(6-(4-(3-(4-Cyano-3-(trifluoromethyl)phenyl)-5,5-dimethyl-4-oxo-2-thioxoimidazolidin-1-yl)-2-fluorobenzamido)hexanamido)-3,3-dimethylbutanoyl)-4-hydroxy-N-(4-(4-methylthiazol-5-yl)benzyl)pyrrolidine-2-carboxamide (11). ¹H NMR (400 MHz, MeOD-d₄) δ=9.20 (s, 1H), 8.16 (d, J=7.2 Hz, 2H), 7.99 (d, J=8.4 Hz, 1H), 7.83 (dd, J=8.0 Hz, J=8.0 Hz, 1H), 7.52-7.40 (m, 4H), 7.35 (dd, J=7.6 Hz, J=8.0 Hz, 2H), 4.63 (s, 1H), 4.59-4.45 (m, 3H), 4.36 (d, J=15.6 Hz, 1H), 3.90 (d, J=10.4 Hz, 1H), 3.83-3.75 (m, 1H), 3.41 (t, J=6.4 Hz, 2H), 2.51 (s, 3H), 2.38-2.15 (m, 3H), 2.13-2.02 (m, 1H), 1.69-1.55 (m, 10H), 1.49-1.26 (m, 2H), 1.03 (s, 9H), UPLC-MS calculated for C₄₈H₅₃F₄N₈O₆S₂ [M+H]⁺: 991.35, found: 977.52. UPLC-retention time: 5.38 min.

(4R)-1-((S)-2-(7-(4-(3-(4-Cyano-3-(trifluoromethyl)phenyl)-5,5-dimethyl-4-oxo-2-thioxoimidazolidin-1-yl)-2-fluorobenzamido)heptanamido)-3,3-dimethylbutanoyl)-4-hydroxy-N-(4-(4-methylthiazol-5-yl)benzyl)pyrrolidine-2-carboxamide (12). ¹H NMR (400 MHz, MeOD-d₄) δ=9.38 (s, 1H), 8.16 (d, J=7.2 Hz, 2H), 7.99 (d, J=8.4 Hz, 1H), 7.82 (dd, J=8.0 Hz, J=7.6 Hz, 1H), 7.54-7.40 (m, 4H), 7.40-7.30 (m, 2H), 4.63 (s, 1H), 4.61-4.47 (m, 3H), 4.37 (d, J=15.6 Hz, 1H), 3.91 (d, J=11.2 Hz, 1H), 3.83-3.77 (m, 1H), 3.40 (t, J=7.2 Hz, 2H), 2.53 (s, 3H), 2.35-2.24 (m, 3H), 2.12-2.05 (m, 1H), 1.69-1.55 (m, 10H), 1.46-1.34 (m, 4H), 1.03 (s, 9H), UPLC-MS calculated for C₄₉H₅₅F₄N₈O₆S₂ [M+H]⁺: 991.36, found: 991.53. UPLC-retention time: 5.55 min.

(4R)-1-((S)-2-(8-(4-(3-(4-Cyano-3-(trifluoromethyl)phenyl)-5,5-dimethyl-4-oxo-2-thioxoimidazolidin-1-yl)-2-fluorobenzamido)octanamido)-3,3-dimethylbutanoyl)-4-hydroxy-N-(4-(4-methylthiazol-5-yl)benzyl)pyrrolidine-2-carboxamide (13). ¹H NMR (400 MHz, MeOD-d₄) δ=9.24 (s, 1H), 8.16 (d, J=7.6 Hz, 2H), 7.99 (d, J=8.4 Hz, 1H), 7.82 (dd, J=8.0 Hz, J=7.6 Hz, 1H), 7.54-7.40 (m, 4H), 7.39-7.30 (m, 2H), 4.64 (s, 1H), 4.62-4.46 (m, 3H), 4.36 (d, J=15.2 Hz, 1H), 3.90 (d, J=10.8 Hz, 1H), 3.83-3.55 (m, 3H), 3.40 (t, J=7.0 Hz, 2H), 2.51 (s, 3H), 2.35-2.17 (m, 3H), 2.12-2.01 (m, 1H), 1.68-1.54 (m, 10H), 1.47-1.32 (m, 6H), 1.02 (s, 9H), UPLC-MS calculated for C₅₀H₅₇F₄N₈O₆S₂ [M+H]⁺: 1005.38, found: 1005.57. UPLC-retention time: 5.74 min.

(4R)-1-((S)-2-(9-(4-(3-(4-Cyano-3-(trifluoromethyl)phenyl)-5,5-dimethyl-4-oxo-2-thioxoimidazolidin-1-yl)-2-fluorobenzamido)nonanamido)-3,3-dimethylbutanoyl)-4-hydroxy-N-(4-(4-methylthiazol-5-yl)benzyl)pyrrolidine-2-carboxamide (14). ¹H NMR (400 MHz, MeOD-d₄) δ=8.97 (s, 1H), 8.16 (d, J=8.0 Hz, 2H), 7.98 (d, J=8.0 Hz, 1H), 7.82 (dd, J=8.0 Hz, J=7.6 Hz, 1H), 7.49-7.38 (m, 4H), 7.40-7.30 (m, 2H), 4.64 (s, 1H), 4.61-4.47 (m, 3H), 4.35 (d, J=15.6 Hz, 1H), 3.91 (d, J=10.8 Hz, 1H), 3.83-3.75 (m, 1H), 3.40 (t, J=7.2 Hz, 2H), 2.48 (s, 3H), 2.35-2.15 (m, 3H), 2.13-2.05 (m, 1H), 1.67-1.56 (m, 10H), 1.45-1.30 (m, 8H), 1.03 (s, 9H), UPLC-MS calculated for C₅₁H₅₉F₄N₈O₆S₂ [M+H]⁺: 1019.39, found: 1019.42. UPLC-retention time: 5.93 min.

(4R)-1-((S)-2-(10-(4-(3-(4-Cyano-3-(trifluoromethyl)phenyl)-5,5-dimethyl-4-oxo-2-thioxoimidazolidin-1-yl)-2-fluorobenzamido)decanamido)-3,3-dimethylbutanoyl)-4-hydroxy-N-(4-(4-methylthiazol-5-yl)benzyl)pyrrolidine-2-carboxamide (15). ¹H NMR (400 MHz, MeOD-d₄) δ=8.90 (s, 1H), 8.16 (d, J=7.6 Hz, 2H), 8.03-7.94 (m, 1H), 7.85-7.78 (m, 1H), 7.50-7.30 (m, 6H), 4.63 (s, 1H), 4.61-4.47 (m, 3H), 4.35 (d, J=15.2 Hz, 1H), 3.90 (d, J=11.6 Hz, 1H), 3.83-3.77 (m, 1H), 3.40 (t, J=7.0 Hz, 2H), 2.47 (s, 3H), 2.32-2.18 (m, 3H), 2.14-2.03 (m, 1H), 1.70-1.55 (m, 10H), 1.47-1.25 (m, 10H), 1.03 (s, 9H). UPLC-MS calculated for C₅₂H₆₁F₄N₈O₆S₂ [M+H]⁺: 1033.41, found: 1033.41. UPLC-retention time: 6.15 min.

(4R)-1-((S)-2-(11-(4-(3-(4-Cyano-3-(trifluoromethyl)phenyl)-5,5-dimethyl-4-oxo-2-thioxoimidazolidin-1-yl)-2-fluorobenzamido)undecanamido)-3,3-dimethylbutanoyl)-4-hydroxy-N-(4-(4-methylthiazol-5-yl)benzyl)pyrrolidine-2-carboxamide (16). ¹H NMR (400 MHz, MeOD-d₄) δ=8.98 (s, 1H), 8.15 (d, J=8.4 Hz, 2H), 8.03-7.94 (m, 1H), 7.87-7.75 (m, 1H), 7.49-7.35 (m, 4H), 7.36-7.31 (m, 2H), 4.63 (s, 1H), 4.60-4.46 (m, 3H), 4.35 (d, J=15.6 Hz, 1H), 3.90 (d, J=11.2 Hz, 1H), 3.83-3.55 (m, 3H), 3.39 (t, J=7.0 Hz, 2H), 2.48 (s, 3H), 2.32-2.16 (m, 3H), 2.13-2.04 (m, 1H), 1.68-1.54 (m, 10H), 1.46-1.30 (m, 12H), 1.03 (s, 9H), UPLC-MS calculated for C₅₃H₆₃F₄N₈O₆S₂ [M+H]⁺: 1047.42, found: 1047.43. UPLC-retention time: 6.38 min.

(4R)-1-((S)-2-(12-(4-(3-(4-Cyano-3-(trifluoromethyl)phenyl)-5,5-dimethyl-4-oxo-2-thioxoimidazolidin-1-yl)-2-fluorobenzamido)dodecanamido)-3,3-dimethylbutanoyl)-4-hydroxy-N-(4-(4-methylthiazol-5-yl)benzyl)pyrrolidine-2-carboxamide (17). ¹H NMR (400 MHz, MeOD-d₄) δ 9.30 (s, 1H), 8.20-8.12 (m, 2H), 8.00 (dd, J=8.3, 1.7 Hz, 1H), 7.85 (t, J=8.0 Hz, 1H), 7.54-7.45 (m, 4H), 7.40-7.34 (m, 2H), 4.65 (s, 1H), 4.57 (dt, J=24.3, 10.3 Hz, 3H), 4.38 (d, J=15.6 Hz, 1H), 3.93 (d, J=11.1 Hz, 1H), 3.82 (dd, J=11.0, 3.8 Hz, 1H), 3.42 (t, J=7.0 Hz, 2H), 2.54 (d, J=3.4 Hz, 3H), 2.41-2.17 (m, 4H), 2.09 (ddd, J=19.6, 10.0, 5.4 Hz, 1H), 1.67-1.62 (m, 3H), 1.61 (s, 6H), 1.36 (d, J=24.1 Hz, 14H), 1.05 (s, 9H). UPLC-MS calculated for C₅₄H₆₅F₄N₈O₆S₂ [M+H]⁺: 1061.44, found 1061.40. UPLC-retention time: 6.5 min. General Procedure for Synthesis of Compound 18.

DIPEA (5 eq.) and HATU (1.2 eq.) were added to a solution of compound 50 (45.1 mg, 0.1 mmol) and tert-butyl 3-(2-(2-aminoethoxy)ethoxy)propanoate (1.1 eq.) in DMF (2 mL). After 30 min at rt, the mixture was subject to HPLC purification to afford the tert-butyl protected compound (53) with 92% yield. Then, compound 53 was obtained by a deprotection reaction in TFA/DCM solvent.

DIPEA (5 eq.) and HATU (1.2 eq.) were added to a solution of 53 (48.8 mg, 0.08 mmol) and 49a (1.1 eq.) in DMF (2 mL). After 30 min at rt, the mixture was subject to HPLC purification to afford compound 18 with 85% yield.

(4R)-1-((S)-13-(tert-Butyl)-1-(4-(3-(4-cyano-3-(trifluoromethyl)phenyl)-5,5-dimethyl-4-oxo-2-thioxoimidazolidin-1-yl)-2-fluorophenyl)-1,11-dioxo-5,8-dioxa-2,12-diazatetradecan-14-oyl)-4-hydroxy-N-(4-(4-methylthiazol-5-yl)benzyl)pyrrolidine-2-carboxamide (18). ¹H NMR (400 MHz, MeOD-d₄) δ=9.76 (s, 1H), 8.16 (d, J=8.4 Hz, 2H), 7.99 (d, J=8.4 Hz, 1H), 7.86 (dd, J=8.4 Hz, J=8.0 Hz, 1H), 7.57-7.45 (m, 4H), 7.41-7.31 (m, 2H), 4.65 (s, 1H), 4.60-4.46 (m, 3H), 4.36 (d, J=15.6 Hz, 1H), 3.92-3.56 (m, 12H), 2.57 (s, 3H), 2.58-2.41 (m, 2H), 2.22-2.00 (m, 2H), 1.59 (s, 6H), 1.39-1.32 (m, 3H), 1.02 (s, 9H). UPLC-MS calculated for C₄₉H₅₅F₄N₈O₈S₂ [M+H]⁺: 1023.35 found 1023.50. UPLC-retention time: 5.2 min.

### General Procedure for Synthesis of Compound 19.

n-BuLi (1 eq.) was added to a solution of compound 56 (2.5 g, 10 mmol) in THE at −78° C. Then, compound 55 (1 eq.) in THE was added at −78° C. slowly. After 2 h at rt, the reaction mixture was quenched with ice/H₂O and extracted with DCM. The organic layer was separated, washed with brine, dried, and evaporated. The final compound 57 was obtained by flash column chromatography (hexane:EtOAc=2:1) with 70% yield.

Compound 57 was placed in a 25 mL round bottomed flask (2.7 g, 7 mmol) with 4-iodoaniline (1 eq.), CuI (0.2 eq.), PdCl₂(PPh₃)₂(0.1 eq.) in DMF and TEA under Ar. Then the mixture was stirred for 4 h at 100° C. After this time, H₂O was added into the resulting complex which was extracted with EtOAc three times. The organic layer was again washed with H₂O before being dried over MgSO₄ and the solvent removed under vacuum, leaving the crude product. The pure product 58 was obtained by flash column chromatography (hexane:EtOAc=4:1) with 80% yield.

A solution of compound 58 (2.1 g, 5.6 mmol) in 2-hydroxy-2-methylpropanenitrile was refluxed for 8 h. The intermediate 59 was obtained by removing the solvent under reduced pressure and used in the next step without further purification.

A solution of compound 59 (2.2 g, 5 mmol) and 4-isothiocyanato-2-(trifluoromethyl)-benzonitrile (1.1 eq.) in 20 mL DMF was stirred at 80° C. for 8 h. Then, 10 mL MeOH and 10 mL 2N HCl were added and the mixture was refluxed for another 4 h. After UPLC-MS demonstrated the full conversion of starting materials, the reaction mixture was cooled to rt and H₂O was added into the mixture. The aqueous layer was extracted with EtOAc, and the combined organic layers were washed with brine, then dried over anhydrous Na₂SO₄. The solvent was removed on a rotary evaporator and the residue was purified by flash column chromatography to afford compound 60 in 60% yield.

A solution of compound 60 in 1:1 TFA/DCM was stirred at rt for 30 min. The solvents were evaporated under reduced pressure to give the corresponding deprotected intermediate 61 (TFA salt) that was used in the following reactions without further purification (95% yield).

DIPEA (5 eq.) and HATU (1.2 eq.) were added to a solution of 61 (62 mg, 0.1 mmol) and compound 49a (1.1 eq.) in DMF (2 mL). After 30 min at rt, the mixture was subject to HPLC purification to afford compound 19 in 90% yield.

(4R)-1-((S)-2-(7-(5-((4-(3-(4-Cyano-3-(trifluoromethyl)phenyl)-5,5-dimethyl-4-oxo-2-thioxoimidazolidin-1-yl)phenyl)ethynyl)pyridin-2-yl)heptanamido)-3,3-dimethylbutanoyl)-4-hydroxy-N-(4-(4-methylthiazol-5-yl)benzyl)pyrrolidine-2-carboxamide (19). ¹H NMR (400 MHz, MeOD-d₄) δ 8.97 (s, 1H), 8.37-8.33 (m, 1H), 8.24-8.14 (m, 3H), 8.02 (dd, J=8.3, 2.0 Hz, 1H), 7.75 (dd, J=8.8, 2.3 Hz, 1H), 7.67 (dd, J=9.1, 2.5 Hz, 2H), 7.51-7.42 (m, 7H), 6.95 (d, J=8.9 Hz, 1H), 4.61-4.53 (m, 3H), 4.39 (d, J=15.6 Hz, 1H), 3.96 (d, J=11.0 Hz, 1H), 3.87-3.82 (m, 1H), 3.70-3.57 (m, 2H), 3.21 (d, J=7.6 Hz, 3H), 2.72 (s, 5H), 2.49 (d, J=2.2 Hz, 3H), 2.41 (t, J=6.7 Hz, 2H), 2.29-2.21 (m, 1H), 2.12 (td, J=9.1, 4.6 Hz, 1H), 1.82-1.74 (m, 3H), 1.60 (s, 6H), 1.08 (s, 9H). UPLC-MS calculated for C₅₅H₅₈F₃N₈O₅S₂ [M+H]⁺: 1031.39, found 1031.48. UPLC-retention time: 5.1 min

### General Procedure for Synthesis of Compounds 20-24.

A solution of 61 (3.43 g, 10 mmol), 4-methylthiazole (2 eq.), KOAc (2 eq.) and Pd(OAc)₂ (1%) in DMF/TEA was stirred at 80° C. for 4 h. After the reaction was complete, the TEA was removed under reduced pressure then H₂O was added into the mixture, and the mixture was extracted 3 times by EtOAc. The solvent was collected, dried with Na₂SO₄ and evaporated under reduced pressure to give the corresponding intermediate 63 by flash column chromatography (hexane/EtOAc=4:1) with 80% yield.

To a solution of 64 (1.41 mg, 10 mmol) in MeOH was added H₂SO₄ (1 eq.). Then the mixture was stirred at 70° C. for 4 h. The mixture was quenched with H₂O and extracted with EtOAc three times. The organic layer was washed with H₂O, brine and dried with Na₂SO₄. The product 65 was obtained by removing the solvent and used without further purification.

t-BuOK (1.5 eq.) was added slowly to a solution of 65 (1.55 g, 10 mmol) in THF, then 2-iodopropane (1.3 eq.) was added dropwise at 0° C. After the addition was completed, the mixture was stirred at rt overnight. After UPLC-MS demonstrated the full conversion of the starting materials, the solvent was removed on a rotary evaporator and the residue was purified by flash column chromatography (hexane/EtOAc=6:1). The desired intermediate 66 was obtained through the hydrolysis by LiOH in THF/H₂O (82% yield).

DIPEA (6 equiv) and HATU (1.2 equiv) were added to a solution of 66 (1.46 g, 8 mmol) and benzyl (2S,4R)-4-hydroxypyrrolidine-2-carboxylate hydrochloride (1.1 eq.) in DMF. The mixture was stirred at rt overnight, then the desired intermediate 70 was isolated according to published method.³⁷

Pd—C(10%) was added under H₂ to a solution of 67 (386 mg, 1 mmol) in MeOH and stirred at rt for 2 h. Then the solvent was removed to afford the product 68 without further purification.

Compound 69 (1.17 g, 10 mmol), 70 (1 eq.), CuI (0.2 eq.), PdCl₂(PPh₃)₂(0.1 eq.) in DMF and TEA solvent were placed in a 25 mL round bottomed flask under Ar. Then the mixture was stirred for 4 h at 100° C. After this time, H₂O was added into the resulting complex and extracted with EtOAc three times. The organic layer was again washed with H₂O before being dried over MgSO₄ and the solvent removed under vacuum leaving the crude product. The pure product 71 was obtained by flash column chromatography (hexane/EtOAc=4:1) with 30% yield.

A solution of 71, tert-butyl 4-((4-aminophenyl)ethynyl)piperidine-1-carboxylate (1.1 g, 3 mmol) in 2-hydroxy-2-methylpropanenitrile was refluxed for 8 h. The intermediate (72) was obtained by removing the solvent under reduced pressure and was used in the next step without further purification.

A solution of 72 (1.3 g, 3 mmol) and 4-isothiocyanato-2-(trifluoromethyl)benzonitrile (1.1 eq.) in 20 mL DMF was stirred at 80° C. for 8 h. Then, 10 mL MeOH and 10 mL 2N HCl were added and the mixture was refluxed for another 4 h. After UPLC-MS demonstrated the full conversion of the starting materials, the reaction mixture was cooled to rt and H₂O was added into the mixture. The aqueous layer was extracted with EtOAc, the combined organic layers were washed with brine, then dried over anhydrous Na₂SO₄. The solvent was removed on a rotary evaporator and the residue was purified by flash column chromatography. The desired intermediate (73) was isolated in 80% yield by deprotection in TFA/DCM.

K₂CO₃ (1.2 equiv) and KI (0.2 equiv) were added to a solution of the intermediate 73 (57.4 mg, 0.1 mmol) and tert-butyl 2-bromoacetate (1.2 eq.) in CH₃CN. After stirring the mixture overnight at 100° C., the solvents were evaporated under reduced pressure to afford the corresponding crude compound that was purified by flash column chromatography (DCM:MeOH=20:1). Compound 74 was obtained through deprotection by TFA in DCM (75% yield).

DIPEA (5 eq.) and HATU (1.2 eq.) were added to a solution of 74 (45 mg, 0.075 mmol) and compound 49a (1.1 eq.) in DMF (2 mL). After 30 min at rt, the mixture was subject to HPLC purification to afford compound 24 with 88% yield. Following the procedures used to prepare 24, compounds 20-23 with different chain lengths were obtained by the same methods.

(4R)-1-((S)-2-(2-(4-(5-((4-(3-(4-Cyano-3-(trifluoromethyl)phenyl)-5,5-dimethyl-4-oxo-2-thioxoimidazolidin-1-yl)phenyl)ethynyl)pyridin-2-yl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (24). ¹H NMR (400 MHz, MeOD-d₄) δ 10.00-9.92 (m, 1H), 8.36-8.29 (m, 1H), 8.23-8.10 (m, 3H), 8.10-7.96 (m, 2H), 7.74 (dd, J=8.5, 2.6 Hz, 2H), 7.60-7.47 (m, 6H), 7.42 (dd, J=6.6, 3.5 Hz, 1H), 5.07 (d, J=6.3 Hz, 1H), 4.68 (s, 1H), 4.65-4.58 (m, 1H), 4.49 (s, 1H), 4.23-4.05 (m, 4H), 3.95 (d, J=11.0 Hz, 1H), 3.78 (dd, J=11.2, 4.2 Hz, 1H), 3.64 (s, 2H), 3.02 (s, 1H), 2.89 (s, 1H), 2.63 (s, 3H), 2.26 (dd, J=13.2, 7.8 Hz, 1H), 2.06-1.94 (m, 1H), 1.61 (s, 6H), 1.54 (d, J=7.0 Hz, 3H), 1.39 (dd, J=6.7, 3.5 Hz, 3H), 1.09 (d, J=9.9 Hz, 9H). UPLC-MS calculated for C₅₅H₅₈F₃N₁₀O₅S₂ [M+H]⁺: 1059.40, found 1059.45. UPLC-retention time: 5.2 min.

(4R)-1-((S)-2-(4-(4-(5-((4-(3-(4-Cyano-3-(trifluoromethyl)phenyl)-5,5-dimethyl-4-oxo-2-thioxoimidazolidin-1-yl)phenyl)ethynyl)pyridin-2-yl)piperazin-1-yl)butanamido)-3,3-dimethylbutanoyl)-4-hydroxy-N-(4-(4-methylthiazol-5-yl)benzyl)pyrrolidine-2-carboxamide (20). ¹H NMR (400 MHz, DMSO-d₆) δ=9.0 (s, 1H, CONH), 8.6 (t, J=4.0 Hz, 1H), 8.4 (m, 2H), 8.3 (m, 2H), 8.10 (m, 2H), 7.8 (m, 1H), 7.7 (m, 1H), 7.6 (m, 1H), 7.4 (m, 4H), 7.2 (s, 1H), 7.0 (s, CONH, 1H), 4.5 (d, 1H), 4.4 (d, 1H), 4.3 (m, 4H), 4.2 (m, 2H), 3.2 (m, 3H), 3.1 (m, 3H), 2.7 (m, 1H), 2.4 (s, 3H), 2.3 (m, 1H), 2.1 (m, 1H), 1.9 (m, 3H), 1.5 (s, 6H), 1.5 (s, OH, 1H), 1.3 (m, 2H), 1.2 (m, 1H), 0.9 (s, t-butyl, 9H). UPLC-MS calculated for C₅₆H₆₀F₃N₁₀O₅S₂ [M+H]⁺: 1073.41, found 1073.54. UPLC-retention time: 5.0 min.

(4R)-1-((S)-2-(4-(4-(5-((4-(3-(4-Cyano-3-(trifluoromethyl)phenyl)-5,5-dimethyl-4-oxo-2-thioxoimidazolidin-1-yl)phenyl)ethynyl)pyridin-2-yl)piperazin-1-yl)butanamido)-3,3-dimethylbutanoyl)-4-hydroxy-N—((S)-1-(4-(4-methylthiazol-5-yl)phenyl)ethyl)pyrrolidine-2-carboxamide (21). ¹H NMR (400 MHz, MeOD-d₄) δ 9.00-8.93 (m, 1H), 8.38 (d, J=2.3 Hz, 1H), 8.18 (d, J=8.8 Hz, 2H), 8.02 (dd, J=8.3, 1.9 Hz, 1H), 7.79 (dt, J=8.9, 1.9 Hz, 1H), 7.67 (dd, J=8.5, 5.4 Hz, 2H), 7.50-7.41 (m, 6H), 6.99 (d, J=9.0 Hz, 1H), 5.04 (d, J=6.9 Hz, 11H), 4.61-4.53 (m, 3H), 4.45 (d, J=4.8 Hz, 1H), 3.95 (d, J=10.9 Hz, 1H), 3.78-3.63 (m, 3H), 3.53-3.46 (m, 1H), 3.28-3.14 (m, 5H), 2.70-2.57 (m, 2H), 2.51 (t, J=2.5 Hz, 3H), 2.24-2.16 (m, 1H), 2.14-1.93 (m, 4H), 1.61 (d, J=3.6 Hz, 6H), 1.54 (d, J=6.9 Hz, 3H), 1.39 (dd, J=7.0, 3.6 Hz, 1H), 1.08 (d, J=17.4 Hz, 9H). UPLC-MS calculated for C₅₇H₆₂F₃N₁₀O₅S₂ [M+H]⁺: 1087.43, found 1087.55. UPLC-retention time: 5.2 min.

(2S,4R)-1-((S)-2-(5-(4-(5-((4-(3-(4-Cyano-3-(trifluoromethyl)phenyl)-5,5-dimethyl-4-oxo-2-thioxoimidazolidin-1-yl)phenyl)ethynyl)pyridin-2-yl)piperazin-1-yl)pentanamido)-3,3-dimethylbutanoyl)-4-hydroxy-N—((S)-1-(4-(4-methylthiazol-5-yl)phenyl)ethyl)pyrrolidine-2-carboxamide (22). ¹H NMR (400 MHz, MeOD-d₄) δ 9.01 (d, J=2.5 Hz, 1H), 8.37 (d, J=2.3 Hz, 1H), 8.20-8.16 (m, 2H), 8.02 (dd, J=8.3, 2.1 Hz, 1H), 7.82-7.75 (m, 1H), 7.71-7.58 (m, 3H), 7.51-7.41 (m, 7H), 6.99 (d, J=8.9 Hz, 1H), 5.03 (d, J=7.0 Hz, 2H), 4.73-4.52 (m, 4H), 4.47 (s, 1H), 3.93 (d, J=11.1 Hz, 1H), 3.78 (dd, J=11.0, 3.9 Hz, 1H), 3.64 (d, J=12.8 Hz, 1H), 3.52-3.46 (m, 1H), 3.23 (s, 4H), 2.50 (d, J=5.1 Hz, 3H), 2.42 (t, J=6.9 Hz, 2H), 2.22 (dd, J=12.8, 8.3 Hz, 1H), 2.00 (td, J=8.9, 4.6 Hz, 1H), 1.79 (dt, J=27.5, 7.7 Hz, 5H), 1.61 (q, J=2.5 Hz, 6H), 1.53 (d, J=6.9 Hz, 3H), 1.07 (d, J=13.3 Hz, 9H). UPLC-MS calculated for C₅₈H₆₄F₃N₁₀O₅S₂ [M+H]⁺: 1101.45, found 1101.53. UPLC-retention time: 4.9 min.

(4R)-1-((S)-2-(3-(4-(5-((4-(3-(4-Cyano-3-(trifluoromethyl)phenyl)-5,5-dimethyl-4-oxo-2-thioxoimidazolidin-1-yl)phenyl)ethynyl)pyridin-2-yl)piperazin-1-yl)propanamido)-3,3-dimethylbutanoyl)-4-hydroxy-N—((S)-1-(4-(4-methylthiazol-5-yl)phenyl)ethyl)pyrrolidine-2-carboxamide (23). ¹H NMR (400 MHz, MeOD-d₄) δ 8.89 (d, J=9.1 Hz, 1H), 8.53 (d, J=7.5 Hz, 1H), 8.38 (d, J=2.3 Hz, 1H), 8.19 (d, J=1.6 Hz, 2H), 8.02 (dt, J=8.2, 2.2 Hz, 1H), 7.78 (dd, J=8.8, 2.3 Hz, 1H), 7.69-7.66 (m, 2H), 7.44 (dd, J=9.2, 3.0 Hz, 6H), 6.98 (d, J=8.9 Hz, 1H), 5.04 (t, J=7.1 Hz, 1H), 4.57 (d, J=5.3 Hz, 2H), 4.46 (s, 1H), 3.95 (d, J=11.0 Hz, 1H), 3.78-3.71 (m, 2H), 3.63 (d, J=11.2 Hz, 1H), 3.52-3.46 (m, 1H), 3.30-3.15 (m, 5H), 2.60 (t, J=6.3 Hz, 2H), 2.50 (d, J=1.9 Hz, 3H), 2.21 (dd, J=13.1, 7.6 Hz, 1H), 2.09 (t, J=6.9 Hz, 2H), 1.98 (td, J=9.2, 4.6 Hz, 1H), 1.61 (d, J=3.6 Hz, 6H), 1.53 (d, J=7.0 Hz, 3H), 1.39 (dd, J=6.7, 3.5 Hz, 1H), 1.10 (s, 9H). UPLC-MS calculated for C₅₆H₆₀F₃N₁₀O₅S₂ [M+H]⁺: 1073.41, found 1073.49. UPLC-retention time: 4.8 min.

### General Procedure for Synthesis of Compound 25.

Triphosgene (1 eq.) was added to a solution of compound 49b (44.4 ng, 0.1 mmol) in DCM and DIPEA (4 eq.) at it. The reaction mixture was stirred at rt for 30 min, and then compound 73 (1 eq.) was added. After an additional 1 h at rt, the mixture was subject to HPLC purification to afford compound 25 with 83% yield.

4-(5-((4-(3-(4-Cyano-3-(trifluoromethyl)phenyl)-5,5-dimethyl-4-oxo-2-thioxoimid-azolidin-1-yl)phenyl)ethynyl)pyridin-2-yl)-N-((2S)-1-((4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)piperazine-1-carboxamide (25). ¹H NMR (400 MHz, MeOD-d₄) δ 9.01 (s, 1H), 8.29 (d, J=2.3 Hz, 1H), 8.23-8.16 (m, 2H), 8.02 (dd, J=8.3, 2.0 Hz, 1H), 7.92 (dt, J=9.3, 2.5 Hz, 1H), 7.75-7.67 (m, 2H), 7.55 (d, J=8.5 Hz, 1H), 7.50-7.42 (m, 5H), 7.15 (dd, J=9.4, 3.5 Hz, 1H), 6.84 (dd, J=8.5, 2.3 Hz, 1H), 5.04 (d, J=7.0 Hz, 1H), 4.60 (d, J=8.5 Hz, 1H), 4.47 (d, J=4.8 Hz, 1H), 3.95 (d, J=11.2 Hz, 1H), 3.78 (q, J=3.9 Hz, 4H), 3.69 (d, J=5.0 Hz, 3H), 3.65-3.59 (m, 1H), 3.23 (d, J=7.4 Hz, 11H), 2.51 (s, 2H), 2.26-2.18 (m, 1H), 1.99 (ddd, J=13.1, 9.0, 4.4 Hz, 1H), 1.61 (s, 6H), 1.54 (d, J=7.1 Hz, 3H), 1.39 (dd, J=6.7, 3.5 Hz, 3H), 1.20 (t, J=7.1 Hz, 1H), 1.09 (s, 9H). UPLC-MS calculated for C₅₄H₅₆F₃N₁₀O₅S₂ [M+H]⁺: 1045.38, found 1045.42. UPLC-retention time: 6.4 min.

### General Procedure for Synthesis of Compound 26.

DIPEA (5 eq.) and HATU (1.2 eq.) were added to a solution of compound 50 (45.1 mg, 0.1 mmol) and tert-butyl-(9-aminononyl)carbamate (1.1 eq.) in DMF (2 mL). After 30 min at rt, the mixture was subject to HPLC purification to afford Boc protected compound 54 with 90% yield. Then, compound 54 was obtained by a deprotection reaction in TFA/DCM solvent.

DIPEA (5 eq.) and HATU (1.2 eq.) were added to a solution of the 54 (47 mg, 0.08 mmol) and 63 (1.1 eq.) in DMF (2 mL). After 30 min at rt, the mixture was subject to HPLC purification to afford compound (S)—N-(9-(3-amino-3-(4-(4-methylthiazol-5-yl)phenyl)propanamido)nonyl)-4-(3-(4-cyano-3-(trifluoromethyl)phenyl)-5,5-dimethyl-4-oxo-2-thioxoimidazolidin-1-yl)-2-fluorobenzamide with 80% yield after deprotection in TFA/DCM.

DIPEA (5 eq.) and HATU (1.2 eq.) were added to a solution of the (S)—N-(9-(3-amino-3-(4-(4-methylthiazol-5-yl)phenyl)propanamido)nonyl)-4-(3-(4-cyano-3-(trifluoromethyl)phenyl)-5,5-dimethyl-4-oxo-2-thioxoimidazolidin-1-yl)-2-fluorobenzamide (66 mg, 0.08 mmol) and 68 (1.1 eq.) in DMF (2 mL). After 30 min at rt, the mixture was subject to HPLC purification to afford compound 26 with 84% yield.

(2S,4R)—N—((S)-3-((9-(4-(3-(4-Cyano-3-(trifluoromethyl)phenyl)-5,5-dimethyl-4-oxo-2-thioxoimidazolidin-1-yl)-2-fluorobenzamido)nonyl)amino)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (26). ¹H NMR (400 MHz, MeOD-d₄) δ 9.78 (s, 1H), 8.26-8.15 (m, 2H), 8.02 (dd, J=8.3, 2.0 Hz, 1H), 7.85 (t, J=8.1 Hz, 1H), 7.56 (s, 4H), 7.40 (d, J=10.1 Hz, 1H), 6.24 (s, 1H), 5.37 (dd, J=8.0, 6.0 Hz, 1H), 4.49 (dd, J=17.4, 9.4 Hz, 2H), 3.97-3.71 (m, 2H), 3.67-3.57 (m, 1H), 3.41 (t, J=7.0 Hz, 3H), 3.11 (td, J=6.8, 3.9 Hz, 2H), 3.02 (s, 1H), 2.94-2.69 (m, 3H), 2.60 (s, 3H), 2.43 (q, J=9.4, 8.6 Hz, 1H), 2.27 (s, 2H), 2.18 (t, J=10.8 Hz, 11H), 1.98 (ddd, J=13.1, 8.5, 5.1 Hz, 11H), 1.61 (s, 6H), 1.47-1.33 (m, 6H), 1.25 (d, J=31.7 Hz, 6H), 1.07 (d, J=6.5 Hz, 3H), 0.89 (dd, J=12.4, 6.6 Hz, 3H). UPLC-MS calculated for C₅₆H₆₄F₄N₉O₇S₂ [M+H]⁺: 1114.43, found 1114.49. UPLC-retention time: 6.0 min.

### General Procedure for Synthesis of Compounds 27-28.

K₂CO₃ (1.2 equiv) and KI (0.2 equiv) were added to a solution of the intermediate 73 (57.4 mg, 0.1 mmol) and tert-butyl (3-bromopropyl)carbamate (1.2 eq.) in CH₃CN was added. After stirring the mixture overnight at 100° C., the solvents were evaporated under reduced pressure to afford the corresponding crude compound that was purified by flash column chromatography (DCM:MeOH=20:1). Then 4-(3-(4-((6-(4-(3-aminopropyl)piperazin-1-yl)pyridin-3-yl)ethynyl)phenyl)-4,4-dimethyl-5-oxo-2-thioxoimidazolidin-1-yl)-2-(trifluoromethyl)benzonitrile was obtained through deprotection by TFA in DCM (72% yield).

DIPEA (5 eq.) and HATU (1.2 eq.) were added to a solution of the compound 4-(3-(4-((6-(4-(3-aminopropyl)piperazin-1-yl)pyridin-3-yl)ethynyl)phenyl)-4,4-dimethyl-5-oxo-2-thioxoimidazolidin-1-yl)-2-(trifluoromethyl)benzonitrile (44 mg, 0.07 mmol) and 63 (1.1 eq.) in DMF (2 mL). After 30 min at rt, the mixture was subject to HPLC purification to afford compound (S)-3-amino-N-(3-(4-(5-((4-(3-(4-cyano-3-(trifluoromethyl)phenyl)-5,5-dimethyl-4-oxo-2-thioxoimidazolidin-1-yl)phenyl)ethynyl)pyridin-2-yl)piperazin-1-yl)propyl)-3-(4-(4-methylthiazol-5-yl)phenyl)propanamide in 81% yield after deprotection with TFA/DCM.

DIPEA (5 eq.) and HATU (1.2 eq.) were added to a solution of the (S)-3-amino-N-(3-(4-(5-((4-(3-(4-cyano-3-(trifluoromethyl)phenyl)-5,5-dimethyl-4-oxo-2-thioxoimidazolidin-1-yl)phenyl)ethynyl)pyridin-2-yl)piperazin-1-yl)propyl)-3-(4-(4-methylthiazol-5-yl)phenyl)propanamide (44 mg, 0.05 mmol) and 68 (1.1 eq.) in DMF (2 mL). After 30 min at rt, the mixture was subject to HPLC purification to afford compound 27 with 84% yield. Following the procedures used to prepare compound 27, compound 28 with different chain lengths was obtained with the same methods.

(2S,4R)—N—((S)-3-((3-(4-(5-((4-(3-(4-Cyano-3-(trifluoromethyl)phenyl)-5,5-dimethyl-4-oxo-2-thioxoimidazolidin-1-yl)phenyl)ethynyl)pyridin-2-yl)piperazin-1-yl)propyl)amino)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (27). ¹H NMR (400 MHz, MeOD-d₄) δ 9.02-8.94 (m, 1H), 8.38 (t, J=4.2 Hz, 1H), 8.22-8.14 (m, 2H), 8.02 (dd, J=8.2, 2.0 Hz, 1H), 7.79 (td, J=8.4, 2.4 Hz, 1H), 7.73-7.67 (m, 2H), 7.64 (s, 1H), 7.58-7.42 (m, 4H), 6.98 (dd, J=26.1, 8.9 Hz, 1H), 5.44-5.32 (m, 1H), 4.72-4.66 (m, 1H), 4.60 (s, 1H), 4.49 (s, 1H), 3.89 (d, J=11.0 Hz, 1H), 3.77 (dd, J=11.0, 4.2 Hz, 1H), 3.68-3.49 (m, 2H), 3.31-2.83 (m, 7H), 2.53 (d, J=5.3 Hz, 3H), 2.30-2.11 (m, 2H), 2.00 (s, 3H), 1.78 (d, J=8.1 Hz, 2H), 1.66 (d, J=6.6 Hz, 1H), 1.61 (s, 6H), 1.48-1.27 (m, 2H), 1.08 (s, 1H), 1.00 (s, 6H). UPLC-MS calculated for C₆₀H₆₃F₃N₁₁O₆S₂ [M+H]⁺: 1154.44, found 1154.56. UPLC-retention time: 5.6 min.

(2S,4R)—N—((S)-3-(4-(5-((4-(((1r,3r)-3-(3-Chloro-4-cyanophenoxy)-2,2,4,4-tetramethylcyclobutyl)carbamoyl)phenyl)ethynyl)pyridin-2-yl)piperazin-1-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (28). ¹H NMR (400 MHz, MeOD-d₄) δ 9.01 (s, 1H), 8.28 (s, 1H), 7.86 (d, J=7.8 Hz, 3H), 7.75 (d, J=8.7 Hz, 1H), 7.65 (d, J=7.9 Hz, 2H), 7.57-7.41 (m, 4H), 7.16 (s, 1H), 7.10 (d, J=8.2 Hz, 11H), 7.01 (d, J=8.8 Hz, 1H), 6.24 (d, J=28.0 Hz, 1H), 5.45 (d, J=26.8 Hz, 1H), 4.61-4.46 (m, 2H), 4.32 (s, 1H), 4.20 (s, 1H), 3.93-3.87 (m, 1H), 3.80-3.48 (m, 10H), 3.07 (ddd, J=22.9, 19.4, 11.3 Hz, 2H), 2.48 (s, 3H), 2.39 (d, J=6.2 Hz, 11H), 2.26 (d, J=6.0 Hz, 3H), 2.05-1.97 (m, 1H), 1.32 (s, 6H), 1.26 (s, 6H), 1.11-1.02 (m, 3H), 0.92-0.83 (m, 3H). UPLC-MS calculated for C₆₀H₆₅ClN₉O₇S [M+H]⁺: 1090.44, found 1090.56. UPLC-retention time: 6.6 min.

### General Procedure for Synthesis of Compounds 29-30.

Compound 75 (2.19 g, 10 mmol), 76 (1.1 eq.), CuI (0.2 eq.), PdCl₂(PPh₃)₂(0.1 eq.) in DMF and TEA were placed in a 25 mL round bottomed flask under Ar. Then the mixture was stirred 4 h at 100° C. After this time, H₂O was added into the resulting complex and extracted with EtOAc three times. The organic layer was again washed with H₂O before being dried over MgSO₄ and the solvent was removed under vacuum leaving the crude product. The pure product (77) was obtained by flash column chromatography (hexane/EtOAc=4:1) with 80% yield.

A solution of 77 (2.4 g, 8 mmol) in 2-hydroxy-2-methylpropanenitrile was refluxed for 8 h. Then by removing the solvent under reduced pressure the intermediate 78 was obtained and used in the next step without further purification.

A solution of 78 (2.9 g, 8 mmol) and 4-isothiocyanato-2-(trifluoromethyl)benzonitrile (1.1 eq.) in 20 mL DMF was stirred at 80° C. for 8 h. Then, 10 mL MeOH and 10 mL 2N HCl were added and the mixture was refluxed for another 4 h. After UPLC-MS demonstrated the full conversion of starting materials, the reaction mixture was cooled to rt and H₂O was added into the mixture. The aqueous layer was extracted with EtOAc, the combined organic layers were washed with brine, then dried over anhydrous Na₂SO₄. The solvent was removed on a rotary evaporator and the residue was purified by flash column chromatography. The desired intermediate 79 was isolated in 85% yield by the deprotection in TFA/DCM.

K₂CO₃ (1.2 equiv) and KI (0.2 equiv) were added to a solution of the intermediate 79 (3 g, 6 mmol) and tert-butyl 4-bromopiperidine-1-carboxylate (1.2 eq.) in CH₃CN. After stirring the mixture overnight at 100° C., the solvents were evaporated under reduced pressure to afford the corresponding crude compound that was purified by flash column chromatography (DCM:MeOH=20:1). Then 80 was obtained through the deprotection by TFA in DCM (82% yield).

DIPEA (5 eq.) and HATU (1.2 eq.) were added to a solution of the compound 80 (57.9 mg, 0.1 mmol) and 63 (1.1 eq.) in DMF (2 mL). After 30 min at rt, the mixture was subject to HPLC purification to afford compound 81 with 85% yield after deprotection in TFA/DCM.

DIPEA (5 eq.) and HATU (1.2 eq.) were added to a solution of the compound 81 (70 mg, 0.85 mmol) and 68 (1.1 eq.) in DMF (2 mL). After 30 min at rt, the mixture was subject to HPLC purification to afford compound 29 with 86% yield. Following the procedures used to prepare compound 29, compound 30 was obtained with the same methods.

(2S,4R)—N—((S)-3-(4-((4-(3-(4-Cyano-3-(trifluoromethyl)phenyl)-5,5-dimethyl-4-oxo-2-thioxoimidazolidin-1-yl)phenyl)ethynyl)-[1,4′-bipiperidin]-1′-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (29). ¹H NMR (400 MHz, MeOD-d₄) δ 9.76 (s, 1H), 8.28-8.16 (m, 2H), 8.01 (dt, J=8.2, 2.6 Hz, 1H), 7.88-7.81 (m, 1H), 7.71-7.50 (m, 5H), 7.47-7.35 (m, 1H), 7.10 (dd, J=61.1, 9.5 Hz, 1H), 6.33-6.21 (m, 1H), 5.56-5.32 (m, 2H), 4.70 (s, 1H), 4.57-4.24 (m, 3H), 4.04-3.41 (m, 7H), 3.29-2.85 (m, 7H), 2.75-2.66 (m, 1H), 2.60 (d, J=5.4 Hz, 2H), 2.48-2.31 (m, 2H), 2.30-2.14 (m, 6H), 2.06-1.77 (m, 4H), 1.66-1.44 (m, 6H), 1.41-1.23 (m, 2H), 1.11-0.99 (m, 3H), 0.87 (qd, J=10.6, 9.5, 5.8 Hz, 3H). UPLC-MS calculated for C₅₈H₆₃F₃N₉O₆S₂ [M+H]⁺: 1102.43, found 1102.37. UPLC-retention time: 4.9 min.

(2S,4R)—N—((S)-3-(4-((4-(7-(6-Cyano-5-(trifluoromethyl)pyridin-3-yl)-8-oxo-6-thioxo-5,7-diazaspiro[3.4]octan-5-yl)phenyl)ethynyl)-[1,4′-bipiperidin]-1′-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (30). ¹H NMR (400 MHz, MeOD-d₄) δ 9.18 (t, J=2.8 Hz, 1H), 8.93 (s, 1H), 8.66 (t, J=2.9 Hz, 1H), 7.89 (t, J=9.4 Hz, 1H), 7.70 (s, 1H), 7.63 (d, J=7.8 Hz, 1H), 7.58-7.38 (m, 5H), 6.26 (d, J=15.8 Hz, 1H), 5.53-5.33 (m, 3H), 4.57-4.41 (m, 2H), 4.30 (s, 2H), 3.95-3.73 (m, 3H), 3.58 (d, J=28.3 Hz, 5H), 3.26-3.07 (m, 5H), 2.95 (d, J=53.1 Hz, 3H), 2.72 (s, 2H), 2.52 (t, J=5.8 Hz, 3H), 2.27 (d, J=6.9 Hz, 3H), 2.19 (d, J=19.2 Hz, 4H), 1.98 (d, J=15.5 Hz, 2H), 1.63 (s, 2H), 1.39 (dd, J=6.7, 3.4 Hz, 1H), 1.32 (d, J=8.2 Hz, 2H), 1.14-1.02 (m, 3H), 0.94-0.84 (m, 3H). UPLC-MS calculated for C₅₈H₆₂F₃N₁₀O₆S₂ [M+H]⁺: 1115.42, found 1115.49. UPLC-retention time: 5.1 min.

(2S,4R)—N-((1S)-3-(4-((4-((3-((4-Cyano-3-(trifluoromethyl)phenyl)amino)-2-hydroxy-2-methyl-3-oxopropyl)sulfinyl)phenyl)ethynyl)-[1,4′-bipiperidin]-1′-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (31). ¹H NMR (400 MHz, MeOD-d₄) δ 8.93 (s, 1H), 8.61 (dd, J=16.4, 8.6 Hz, 1H), 8.26 (s, 1H), 8.07-7.83 (m, 4H), 7.47 (d, J=28.4 Hz, 5H), 6.31-6.20 (m, 1H), 5.45 (d, J=25.2 Hz, 2H), 4.71 (s, 1H), 4.59-4.41 (m, 2H), 4.29 (s, 2H), 4.12 (dd, J=14.8, 1.7 Hz, 1H), 3.96-3.70 (m, 3H), 3.70-3.44 (m, 6H), 3.27-3.05 (m, 4H), 2.95 (s, 2H), 2.70-2.60 (m, 1H), 2.52 (t, J=5.1 Hz, 3H), 2.39 (d, J=32.8 Hz, 2H), 2.27 (d, J=6.3 Hz, 3H), 2.16 (s, 2H), 2.03-1.89 (m, 2H), 1.79-1.66 (m, 1H), 1.58-1.47 (m, 3H), 1.41-1.27 (m, 2H), 1.12-0.96 (m, 3H), 0.89 (s, 3H). UPLC-MS calculated for C₅₆H₆₄F₄N₉O₇S₂[M+H]⁺: 1125.42, found 1125.49. UPLC-retention time: 4.0 min.

### General Procedure for Synthesis of Compound 33.

Compound 99 (1.0 equiv) in acetone was added slowly at 0° C. to a solution of the intermediate 100 (0.4 g, 4 mmol) and Na₂CO₃ (1.5 equiv) in acetone. Then, the mixture was refluxed overnight. After UPLC-MS demonstrated the full conversion of starting materials, the solvents were evaporated under reduced pressure to afford the corresponding crude compound 101 that was purified by flash column chromatography (hexane/EtOAc=4:1) with 95% yield.

A solution of the intermediate 101 (1.02 g, 3.8 mmol) and NH₂NH₂ (1.1 equiv) in EtOH was refluxed for 2 h. After UPLC-MS demonstrated the full conversion of starting materials, the solvents were evaporated under reduced pressure to afford the corresponding crude compound 102 that was purified by flash column chromatography (DCM:MeOH=20:1) with 91% yield.

Compound 85 and 2-chloro-4-fluorobenzonitrile (1.5 eq.) were added at 0° C. to a mixture of 102 (0.8 g, 3 mmol) and NaH (1.2 eq.) in DMF and stirred for 5 min. The mixture was then stirred at rt for 3 h. After UPLC-MS demonstrated the full conversion of starting materials, ice/H₂O and brine were added into the mixture and the organic layer was collected and dried with Na₂SO₄. The solvent was removed on a rotary evaporator giving the desired intermediate (103) which was purified by flash column chromatography (hexane/EtOAc=2:1) in 86% yield.

Compounds 103, 4-(4-(4-bromobenzyl)-3,5-dimethyl-1H-pyrazol-1-yl)-2-chlorobenzonitrile (399 mg, 1 mmol), 92, tert-butyl 4-ethynyl-[1,4′-bipiperidine]-1′-carboxylate (1.1 eq.), CuI (0.2 eq.), PdCl₂(PPh₃)₂(0.1 eq.) in DMF and TEA solvent were placed in a 25 mL round bottomed flask under Ar. Then the mixture was stirred 4 h at 100° C., then H₂O was added into the resulting complex which was extracted with EtOAc three times. The organic layer was again washed with H₂O before being dried over MgSO₄ and the solvent removed under vacuum leaving the crude product. The pure product was obtained by flash column chromatography (DCM:MeOH=20:1). Compound 104 was obtained through deprotection by TFA in DCM (80% yield).

DIPEA (5 eq.) and HATU (1.2 eq.) were added to a solution of compounds 104 (51.1 mg, 0.1 mmol) and 63 (1.1 eq.) in DMF (2 mL). After 30 min at rt, the mixture was subject to HPLC purification to afford compound 105 with 84% yield after deprotection in TFA/DCM.

DIPEA (5 eq.) and HATU (1.2 eq.) were added to a solution of the compounds 105 (60 mg, 0.08 mmol) and 68 (1.1 eq.) in DMF (2 mL). After 30 min at rt, the mixture was subject to HPLC purification to afford compound 33 in 81% yield.

(2S,4R)—N—((S)-3-(4-((4-((1-(3-Chloro-4-cyanophenyl)-3,5-dimethyl-1H-pyrazol-4-yl)methyl)phenyl)ethynyl)-[1,4′-bipiperidin]-1′-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (33). ¹H NMR (400 MHz, MeOD-d₄) δ=8.94 (s, 1H), 7.92 (d, J=8.4 Hz, 2H), 7.84 (s, 1H), 7.64 (d, J=8.4 Hz, 1H), 7.50-7.45 (m, 4H), 7.39-7.26 (m, 2H), 7.14 (dd, J=8.8 Hz, J=8.4 Hz, 2H), 6.22 (s, 1H), 4.46 (s, 1H), 3.85-3.76 (m, 6H), 2.56-2.44 (m, 6H), 2.25 (s, 3H), 2.13 (s, 3H), 1.10-0.95 (m, 6H), 0.94-0.78 (m, 6H), UPLC-MS calculated for C₅₈H₆₅ClN₉O₅S [M+H]⁺: 1034.45, found 1034.65. UPLC-retention time: 5.1 min.

### General Procedure for Synthesis of Compounds 32, 34-39, 43-44.

Compound 82 (2.62 g, 10 mmol), 76 (1.1 eq.), CuI (0.2 eq.), PdCl₂(PPh₃)₂ (0.1 eq.) in DMF and TEA solvent were placed in a 25 mL round bottomed flask under Ar. Then the mixture was stirred for 4 h at 100° C. After this time, H₂O was added into the resulting complex and extracted with EtOAc three times. The organic layer was again washed with H₂O before being dried over MgSO₄ and the solvent removed under vacuum leaving the crude product. The pure product was obtained by flash column chromatography (hexane/EtOAc=4:1). Then, 83 was obtained through deprotection by TFA in DCM with 90% yield.

K₂CO₃ (1.2 equiv) and KI (0.2 equiv) were added to a solution of the deprotected intermediate 83 (1.94 g, 8 mmol), tert-butyl 4-bromopiperidine-1-carboxylate (1.2 equiv) in CH₃CN. After stirring the mixture overnight at 100° C., the solvents were evaporated under reduced pressure to afford the corresponding crude compound 4-((4-(methoxycarbonyl)phenyl)ethynyl)-[1,4′-bipiperidine]-1′-carboxylate which was purified by flash column chromatography (DCM:MeOH=20:1) with 85% yield. NaOH (2 eq.) was added to a solution of tert-butyl 4-((4-(methoxycarbonyl)phenyl)ethynyl)-[1,4′-bipiperidine]-1′-carboxylate in MeOH/H₂O and stirred at rt for 2 h. Then MeOH was removed under reduced pressure, the pH was adjusted with 2N HCl and the mixture was extracted with EtOAc. The solvent was removed to afford the product 84 which was used without further purification.

To a solution of 86 (2.43 g, 10 mmol) in dry DMF was added NaH (1.2 eq.) at 0° C. After stirring the mixture at 0° C. for 20 min, 85 was added and the mixture was stirred at rt for 4 h. After UPLC-MS demonstrated the full conversion of starting materials, H₂O was added and the mixture was extracted with EtOAc, the combined organic layers were washed with brine, then dried over anhydrous Na₂SO₄. The solvent was removed on a rotary evaporator. The desired intermediate 87 was obtained by deprotection with TFA in DCM in 88% yield.

DIPEA (5 eq.) and HATU (1.2 eq.) were added to a solution of the compound 87 (278 mg, 1 mmol) and 84 (1.1 eq.) in DMF (2 mL). After 30 min at rt, the mixture was subjected to HPLC purification to afford compound 88 with 88% yield after deprotection in TFA/DCM.

DIPEA (5 eq.) and HATU (1.2 eq.) were added to a solution of the compound 88 (57.2 mg, 0.1 mmol) and 63 (1.1 eq.) in DMF (2 mL). After 30 min at rt, the mixture was subject to HPLC purification to afford compound 89 with 80% yield after deprotection in TFA/DCM.

DIPEA (5 eq.) and HATU (1.2 eq.) were added to a solution of the compound 89 (40.8 mg, 0.05 mmol) and 68 (1.1 eq.) in DMF (2 mL). After 30 min at rt, the mixture was subject to HPLC purification to afford compound 32 with 83% yield. Following the procedures used to prepare compound 32, compound 34-38 were obtained using the same methods.

(2S,4R)—N—((S)-3-(4-((4-(((1r,3r)-3-(3-Chloro-4-cyanophenoxy)-2,2,4,4-tetramethyl-cyclobutyl)carbamoyl)phenyl)ethynyl)-[1,4′-bipiperidin]-1′-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide (32). ¹H NMR (400 MHz, MeOD-d₄) δ 9.16 (t, J=5.8 Hz, 1H), 7.81 (t, J=8.8 Hz, 2H), 7.74 (d, J=8.7 Hz, 1H), 7.59 (d, J=7.5 Hz, 1H), 7.52 (dd, J=9.1, 5.7 Hz, 5H), 7.14 (d, J=2.1 Hz, 1H), 7.00 (dd, J=8.7, 2.1 Hz, 1H), 6.33-6.20 (m, 1H), 5.53-5.37 (m, 1H), 4.70 (t, J=12.9 Hz, 1H), 4.46 (t, J=18.4 Hz, 2H), 4.31 (s, 2H), 4.17 (s, 1H), 3.90 (dd, J=10.8, 3.8 Hz, 1H), 3.85-3.75 (m, 1H), 3.67-3.51 (m, 4H), 3.28-3.08 (m, 4H), 3.01-2.88 (m, 1H), 2.67 (dd, J=24.1, 11.9 Hz, 1H), 2.56-2.51 (m, 3H), 2.48-2.32 (m, 2H), 2.27 (d, J=4.2 Hz, 3H), 2.25-2.06 (m, 6H), 1.98 (dd, J=10.1, 5.0 Hz, 3H), 1.77 (dd, J=29.2, 14.2 Hz, 1H), 1.62-1.42 (m, 1H), 1.30 (s, 6H), 1.25 (s, 6H), 1.07 (dd, J=13.4, 6.7 Hz, 3H), 0.91-0.84 (m, 3H). UPLC-MS calculated for C₆₁H₇₂ClN₈O₇S [M+H]⁺: 1095.49, found 1095.55. UPLC-retention time: 5.4 min.

(2S,4R)—N—((S)-3-(4-((4-(((1r,3r)-3-(3-Chloro-4-cyanophenoxy)-2,2,4,4-tetramethyl-cyclobutyl)carbamoyl)phenyl)ethynyl)-[1,4′-bipiperidin]-1′-yl)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (34). ¹H NMR (400 MHz, MeOD-d₄) δ 9.19 (s, 1H), 7.81 (dd, J=10.6, 7.6 Hz, 2H), 7.76-7.71 (m, 1H), 7.54 (t, J=12.6 Hz, 6H), 7.15 (d, J=5.2 Hz, 1H), 7.00 (t, J=6.6 Hz, 1H), 5.42 (s, 1H), 4.68 (dd, J=38.4, 26.4 Hz, 3H), 4.48 (s, 1H), 4.31 (d, J=4.9 Hz, 1H), 4.20 (t, J=12.0 Hz, 2H), 3.98-3.75 (m, 2H), 3.51 (s, 4H), 3.23-2.84 (m, 5H), 2.65 (d, J=13.2 Hz, 1H), 2.60-2.52 (m, 3H), 2.20 (dt, J=90.9, 52.2 Hz, 9H), 1.73-1.36 (m, 4H), 1.31 (d, J=5.0 Hz, 6H), 1.25 (d, J=5.7 Hz, 6H), 1.08 (d, J=5.8 Hz, 9H). ¹³C NMR (100 MHz, MeOD-d₄) δ 171.80, 170.39, 170.12, 169.93, 169.04, 162.97, 152.32, 141.82, 137.56, 135.37, 134.11, 133.98, 131.45, 131.26, 129.99, 129.09, 127.35, 126.30, 126.07, 116.61, 116.06, 115.78, 114.31, 113.24, 104.36, 92.08, 90.65, 84.38, 80.80, 79.03, 76.73, 69.56, 63.45, 59.57, 59.51, 59.06, 57.37, 56.75, 50.63, 50.43, 46.07, 44.04, 43.93, 40.34, 39.94, 38.11, 37.95, 37.44, 36.40, 35.86, 29.44, 27.74, 26.49, 26.08, 25.57, 23.99, 23.07, 22.31, 14.09, 12.75, 12.61, 12.51. UPLC-MS calculated for C₆₂H₇₅ClFN₈O₇S [M+H]⁺: 1129.52, found 1129.46. UPLC-retention time: 4.8 min.

(2S,4R)—N—((S)-3-(4-((4-(((1r,3r)-3-(3-Chloro-4-cyanophenoxy)-2,2,4,4-tetramethyl-cyclobutyl)carbamoyl)phenyl)ethynyl)-[1,4′-bipiperidin]-1′-yl)-1-(4-(4-methylthiazol-5-yl)phenyl)-3-oxopropyl)-1-((S)-2-(1-cyanocyclopropane-1-carboxamido)-3,3-dimethylbutanoyl)-4-hydroxypyrrolidine-2-carboxamide (35). ¹H NMR (400 MHz, MeOD-d₄) δ 9.77 (s, 1H), 7.85-7.77 (m, 2H), 7.74 (d, J=8.6 Hz, 1H), 7.64-7.48 (m, 6H), 7.15 (s, 1H), 7.00 (d, J=8.6 Hz, 1H), 5.46 (d, J=5.4 Hz, 1H), 4.68 (d, J=6.0 Hz, 2H), 4.58 (d, J=7.4 Hz, 1H), 4.47 (s, 1H), 4.31 (s, 1H), 4.18 (s, 1H), 3.79 (d, J=12.6 Hz, 2H), 3.58 (d, J=9.4 Hz, 3H), 3.41 (s, 1H), 3.14 (d, J=20.9 Hz, 3H), 2.94 (d, J=6.5 Hz, 1H), 2.88-2.69 (m, 1H), 2.61 (d, J=4.1 Hz, 3H), 2.46-2.04 (m, 7H), 1.99 (d, J=7.0 Hz, 2H), 1.74-1.51 (m, 6H), 1.31 (s, 6H), 1.25 (s, 6H), 1.13-1.03 (m, 9H). UPLC-MS calculated for C₆₃H₇₄ClN₉O₇S [M+H]⁺: 1136.52, found 1136.56. UPLC-retention time: 4.6 min.

(2S,4R)-1-((S)-2-Acetamido-3,3-dimethylbutanoyl)-N—((S)-3-(4-((4-(((1r,3r)-3-(3-chloro-4-cyanophenoxy)-2,2,4,4-tetramethylcyclobutyl)carbamoyl)phenyl)ethynyl)-[1,4′-bipiperidin]-1′-yl)-1-(4-(4-methylthiazol-5-yl)phenyl)-3-oxopropyl)-4-hydroxypyrrolidine-2-carboxamide (36). ¹H NMR (400 MHz, MeOD-d₄) δ 8.96 (s, 1H), 7.88-7.73 (m, 4H), 7.59 (d, J=8.0 Hz, 1H), 7.51 (d, J=6.7 Hz, 6H), 7.15 (d, J=2.6 Hz, 1H), 7.00 (dd, J=8.8, 2.6 Hz, 1H), 5.37 (s, 2H), 4.75-4.44 (m, 9H), 4.31 (s, 1H), 4.25-4.11 (m, 2H), 3.85 (dd, J=48.9, 10.8 Hz, 2H), 3.53 (s, 3H), 3.09 (s, 6H), 2.90 (d, J=13.8 Hz, 1H), 2.64 (d, J=14.0 Hz, 1H), 2.55-2.47 (m, 3H), 2.16 (s, 6H), 2.01 (dd, J=7.3, 3.4 Hz, 3H), 1.61 (s, 1H), 1.31 (s, 6H), 1.25 (s, 6H), 1.05 (d, J=5.2 Hz, 9H). UPLC-MS calculated for C₆₀H₇₄ClN₈O₇S [M+H]⁺: 1085.51, found 1085.62. UPLC-retention time: 5.1 min.

(2S,4R)—N—((R)-3-(4-((4-(((1r,3r)-3-(3-Chloro-4-cyanophenoxy)-2,2,4,4-tetramethyl-cyclobutyl)carbamoyl)phenyl)ethynyl)-[1,4′-bipiperidin]-1′-yl)-1-(4-(4-methylthiazol-5-yl)phenyl)-3-oxopropyl)-1-((S)-2-(1-fluorocyclopropane-1-carboxamido)-3,3-dimethyl-butanoyl)-4-hydroxypyrrolidine-2-carboxamide (37). ¹H NMR (400 MHz, MeOD-d₄) δ 9.56 (s, 1H), 7.81 (dd, J=10.1, 8.1 Hz, 2H), 7.74 (d, J=8.8 Hz, 1H), 7.72-7.64 (m, 2H), 7.60 (d, J=8.1 Hz, 1H), 7.53 (dt, J=16.5, 5.9 Hz, 3H), 7.15 (d, J=2.4 Hz, 1H), 7.00 (dd, J=8.8, 2.5 Hz, 1H), 5.46-5.40 (m, 1H), 4.77-4.49 (m, 4H), 4.31 (d, J=2.4 Hz, 1H), 4.18 (d, J=2.9 Hz, 2H), 3.88-3.74 (m, 2H), 3.57 (s, 4H), 3.21-2.88 (m, 5H), 2.74-2.65 (m, 1H), 2.58 (d, J=5.2 Hz, 3H), 2.41-1.96 (m, 9H), 1.86-1.39 (m, 4H), 1.31 (d, J=2.6 Hz, 6H), 1.25 (s, 6H), 1.06-0.96 (m, 9H). UPLC-MS calculated for C₆₂H₇₄ClFN₈O₇S [M+H]⁺: 1129.52, found 1129.58. UPLC-retention time: 4.9 min.

(2S,4R)—N—((S)-3-(4-((4-(((1r,3r)-3-(3-Chloro-4-cyanophenoxy)-2,2,4,4-tetramethyl-cyclobutyl)carbamoyl)phenyl)ethynyl)-[1,4′-bipiperidin]-1′-yl)-1-(4-(4-methylthiazol-5-yl)phenyl)-3-oxopropyl)-1-((R)-2-(1-fluorocyclopropane-1-carboxamido)-3,3-dimethyl-butanoyl)-4-hydroxypyrrolidine-2-carboxamide (38). ¹H NMR (400 MHz, MeOD-d₄) δ 9.55 (s, 1H), 7.81 (dd, J=12.3, 8.3 Hz, 2H), 7.73 (d, J=8.7 Hz, 1H), 7.63-7.49 (m, 6H), 7.13 (d, J=2.4 Hz, 11H), 6.99 (dt, J=8.9, 1.7 Hz, 11H), 5.44 (d, J=6.6 Hz, 1H), 4.72 (s, 1H), 4.59 (dt, J=17.8, 4.7 Hz, 2H), 4.49 (s, 1H), 4.30 (d, J=2.2 Hz, 1H), 4.17 (d, J=2.7 Hz, 2H), 3.95 (dd, J=10.1, 4.0 Hz, 1H), 3.75 (dt, J=12.5, 4.3 Hz, 1H), 3.56 (s, 3H), 3.40 (s, 1H), 3.22-2.95 (m, 5H), 2.74-2.64 (m, 1H), 2.58 (d, J=5.6 Hz, 3H), 2.36-1.98 (m, 9H), 1.87-1.45 (m, 3H), 1.41-1.34 (m, 2H), 1.30 (d, J=2.5 Hz, 6H), 1.24 (s, 6H), 1.17-1.10 (m, 9H). UPLC-MS calculated for C₆₂H₇₄ClFN₈O₇S [M+H]⁺: 1129.52, found 1129.56. UPLC-retention time: 4.8 min.

### General Procedure for Synthesis of Compound 31.

K₂CO₃ (1.2 equiv) and KI (0.2 equiv) were added to a solution of the intermediate 90 (1 g, 10 mmol) and 91 (1.2 eq.) in CH₃CN. After stirring the mixture overnight at 100° C., the solvents were evaporated under reduced pressure to afford the corresponding crude compound 92 that was purified by flash column chromatography (DCM:MeOH=20:1) with 85% yield.

A solution of 94 (1.3 eq.) in THE was added to a mixture of NaH (1.3 eq.) in THE at 0° C. and stirred for 5 min. Then a solution of 93 (2.7 g, 10 mmol) in THE was added slowly. The mixture was stirred at rt for 3 h. After UPLC-MS demonstrated the full conversion of starting materials, the solvent THE was distilled off and some EtOAc was added and the solution was washed with brine and H₂O. The combined organic layers were dried over anhydrous Na₂SO₄. The solvent was removed on a rotary evaporator giving the desired intermediate 95 which was used without further purification.

Compound 95 (460 mg, 1 mmol) was dissolved with DCM and 30% H₂O₂(8 eq.) was added then the mixture was cooled to −55° C. and trifluoroacetic anhydride (6 eq.) was added very slowly, keeping the reaction temperature below 0° C. After the addition was complete, the reaction mixture was stirred at rt for 16 h. After UPLC-MS demonstrated the full conversion of starting materials, ice/H₂O and brine was added into the mixture which was stirred for another 20 min, then the organic layer was collected and dried with Na₂SO₄. The solvent was removed on a rotary evaporator giving the desired intermediate 96 which was purified by flash column chromatography (hexane/EtOAc=2:1) with 80% yield.

Compounds 96 (390 mg, 0.8 mmol) and 92, and tert-butyl 4-ethynyl-[1,4′-bipiperidine]-1′-carboxylate (1.1 eq.), CuI (0.2 eq.), PdCl₂(PPh₃)₂(0.1 eq.) in DMF and TEA solvent were placed in a 25 mL round bottom flask under Ar. Then the mixture was stirred for 4 h at 100° C. Then H₂O was added into the resulting complex which was extracted with EtOAc three times. The organic layer was again washed with H₂O before being dried over MgSO₄ and the solvent was removed under vacuum leaving the crude product. The pure product was obtained by flash column chromatography (DCM:MeOH=20:1). Then, compound 97 was obtained through the deprotection by TFA in DCM (82% yield).

DIPEA (5 eq.) and HATU (1.2 eq.) were added to a solution of compounds 97 (60.2 mg, 0.1 mmol) and 63 (1.1 eq.) in DMF (2 mL). After 30 min at rt, the mixture was subject to HPLC purification to afford compound 98 in 88% yield after deprotection in TFA/DCM.

DIPEA (5 eq.) and HATU (1.2 eq.) were added to a solution of the compound 98 (67.7 mg, 0.08 mmol) and 68 (1.1 eq.) in DMF (2 mL). After 30 min at rt, the mixture was subject to HPLC purification to afford compound 31 in 83% yield.

### General Procedure for Synthesis of Compound 39.

DIPEA (5 eq.) and HATU (1.2 eq.) were added to a solution of compounds 87 (27.8 mg, 0.1 mmol) and 106 (1.1 eq.) in DMF (2 mL). After 30 min at rt, the mixture was subject to HPLC purification to afford compound 107 in 80% yield.

NaOH (2 eq.) was added to a solution of 107 (35 mg, 0.08 mmol) in MeOH/H₂O and stirred at rt for 2 h. Then the MeOH was removed under reduced pressure, the pH was adjusted to acidity with 2M HCl and the mixture was extracted with EtOAc. The solvent was removed to afford the product 108 which was used without further purification.

DIPEA (5 eq.) and HATU (1.2 eq.) were added to a solution of the compound 108 (33 mg, 0.08 mmol) and tert-butyl (9-aminononyl)carbamate (1.1 eq.) in DMF (2 mL). After 30 min at rt, the mixture was subject to HPLC purification to afford compound 109 with 85% yield.

DIPEA (5 eq.) and HATU (1.2 eq.) were added to a solution of the compound 109 (39 mg, 0.07 mmol) and 63 (1.1 eq.) in DMF (2 mL). After 30 min at rt, the mixture was subject to HPLC purification to afford compound 110 with 84% yield after deprotection in TFA/DCM.

DIPEA (5 eq.) and HATU (1.2 eq.) were added to a solution of the compound 110 (40.5 mg, 0.05 mmol) and 68 (1.1 eq.) in DMF (2 mL). After 30 min at rt, the mixture was subject to HPLC purification to afford compound 39 with 84% yield.

N₁-((1r,3r)-3-(3-Chloro-4-cyanophenoxy)-2,2,4,4-tetramethylcyclobutyl)-N4-(9-((S)-3-((2S,4R)-4-hydroxy-1-((R)-3-methyl-2-(3-methylisoxazol-5-yl)butanoyl)pyrrolidine-2-carboxamido)-3-(4-(4-methylthiazol-5-yl)phenyl)propanamido)nonyl)terephthalamide (39). ¹H NMR (400 MHz, MeOD-d₄) δ 8.95 (s, 1H), 7.99-7.89 (m, 4H), 7.74 (dd, J=8.8, 1.4 Hz, 1H), 7.48 (t, J=4.8 Hz, 4H), 7.15 (t, J=1.9 Hz, 1H), 7.00 (dt, J=8.7, 1.8 Hz, 1H), 6.29-6.18 (m, 1H), 5.41-5.31 (m, 1H), 4.99 (s, 2H), 4.50 (dd, J=17.3, 9.2 Hz, 2H), 4.31 (s, 1H), 4.20 (s, 1H), 3.89 (dd, J=10.7, 4.3 Hz, 1H), 3.83-3.75 (m, 1H), 3.61 (d, J=10.4 Hz, 1H), 3.38 (dd, J=14.0, 7.1 Hz, 7H), 3.16-3.03 (m, 2H), 2.91-2.73 (m, 2H), 2.51 (d, J=1.4 Hz, 3H), 2.42 (dq, J=13.2, 6.8 Hz, 1H), 2.32-2.24 (m, 3H), 2.17 (t, J=10.5 Hz, 1H), 2.00 (dq, J=17.5, 6.5, 5.2 Hz, 1H), 1.68-1.56 (m, 2H), 1.35-1.24 (m, 18H), 1.16 (s, 1H), 1.11-1.04 (m, 3H), 0.93-0.84 (m, 3H). UPLC-MS calculated for C₅₉H₇₄ClN₈O₈S [M+H]⁺: 1089.50, found 1089.57. UPLC-retention time: 6.1 min.

### General Procedure for Synthesis of Compound 40.

DIPEA (5 eq.) and HATU (1.2 eq.) were added to a solution of the compound 108 (42.6 mg, 0.1 mmol) and tert-butyl-(10-aminodecyl)carbamate (1.1 eq.) in DMF (2 mL). After 30 min at rt, the mixture was subject to HPLC purification to afford compound 112 in 80% yield.

DIPEA (5 eq.) was added to a solution of the compound 112 (46.4 mg, 0.08 mmol) and 2-(2,6-dioxopiperidin-3-yl)-4-fluoroisoindoline-1,3-dione (1.1 eq.) in DMSO (2 mL). After 4 h at 80° C., the mixture was subject to HPLC purification to afford compound 40 with 90% yield.

N1-((1r,3r)-3-(3-Chloro-4-cyanophenoxy)-2,2,4,4-tetramethylcyclobutyl)-N4-(10-((2-(2,6-dioxopiperidin-3-yl)-1,3-dioxoisoindolin-4-yl)amino)decyl)terephthalamide (40). ¹H NMR (400 MHz, DMSO-d₆) δ 11.10 (s, 1H), 8.56 (t, J=5.6 Hz, 1H), 8.01-7.87 (m, 7H), 7.64-7.56 (m, 1H), 7.21 (d, J=2.4 Hz, 1H), 7.08 (d, J=8.6 Hz, 1H), 7.04-6.95 (m, 2H), 6.52 (t, J=5.9 Hz, 111), 5.06 (dd, J=12.9, 5.4 Hz, 1H), 4.34 (s, 1H), 4.13-4.08 (m, 1H), 3.30-3.24 (m, 4H), 2.94-2.84 (m, 111), 2.68-2.51 (m, 3H), 2.05 (ddd, J=12.7, 6.9, 2.9 Hz, 1H), 1.56 (dq, J=14.2, 6.7 Hz, 5H), 1.33-1.28 (m, 10H), 1.25 (s, 6H), 1.15 (s, 6H). UPLC-MS calculated for C₄₆H₅₄ClN₆O₇[M+H]⁺: 837.37, found 837.46. UPLC-retention time: 6.8 min.

### General Procedure for Synthesis of Compound 41.

DIPEA (5 eq.) was added to a solution of the compound 88 (57.2 mg, 0.1 mmol) and 2-(2,6-dioxopiperidin-3-yl)-4-fluoroisoindoline-1,3-dione (1.1 eq.) in DMSO (2 mL). After 4 h at 80° C., the mixture was subject to HPLC purification to afford compound 41 in 88% yield.

N-((1r,3r)-3-(3-Chloro-4-cyanophenoxy)-2,2,4,4-tetramethylcyclobutyl)-4-((1′-(2-(2,6-dioxopiperidin-3-yl)-1,3-dioxoisoindolin-4-yl)-[1,4′-bipiperidin]-4-yl)ethynyl)benzamide (41). ¹H NMR (400 MHz, DMSO-d₆) δ=11.1 (br, NH(CO)₂, 1H), 9.37 (s, CONH, 1H), 7.9 (d, 1H), 7.8 (d, 1H), 7.7 (d, 1H), 7.5 (dd, 1H), 7.4 (t, 1H), 7.2 (s, 1H), 7.0 (d, 1H), 6.5 (m, 3H), 5.7 (s, 4H), 5.1 (m, 1H), 4.1 (m, 3H), 3.8 (s, 1H), 3.5 (m, 2H), 3.2 (m, 311), 3.0 (m, 311), 2.6 (m, 1H), 2.2 (m, 3H), 2.1 (m, 2H), 1.9 (m, 2H), 1.2 (m, 12H). UPLC-MS calculated for C₄₇H₅₀ClN₆O₆[M+H]⁺: 829.35, found 829.38. UPLC-retention time: 5.5 min.

### Example 4

### General Pharmacological Methods

Cell lines and Cell Culture: All the LNCaP, VCaP and 22RV1 cells used were purchased from American Type Culture Collection (ATCC). LNCaP and 22RV1 cells were grown in RPMI 1640 (Invitrogen), VCaP cells were grown in DMEM with Glutamax (Invitrogen). All of the cells were supplemented with 10% fetal bovine serum (Invitrogen) at 37° C. in a humidified 5% CO₂ incubator.

Antibodies and Reagents.

| REAGENT or | | |
|---|---|---|
| RESOURCE | SOURCE | IDENTIFIER |
| Antibodies | | |
| GAPDH(14C10) | CST | Cat# 3683S |
| AR | ABCAM | Cat# ab194196 EPR1535(2) |
| Secondary antibody | Invitrogen | Cat# A16172 |
| conjugated with HRP | | |
| Chemicals | | |
| Enzalutamide | 1Click Chemistry | Cat# 915087-33-1 |
| MG132 Proteasome | Selleck Chemicals | Cat# S2619 |
| inhibitor | | |
| PRIMER | | |
| TMPRSS2 qPCR | Applied Biosystems | Cat# Hs01122322_m1 |
| FKBP5 qPCR | Applied Biosystems | Cat# Hs01561006_m1 |
| KLK3 qPCR | Applied Biosystems | Cat# Hs02576345_m1 |
| AR qPCR | Applied Biosystems | Cat# Hs00171172_m1 |
| GAPDH qPCR | Applied Biosystems | Cat# Hs99999905_m1 |
| ERG qPCR | Applied Biosystems | Cat# Hs01554634_m1 |

Cell viability was evaluated by a WST-8 assay (Dojindo) following the manufacturer's instructions. Western blot analysis was performed as previously described.41,42

Quantitative Real-Time Polymerase Chain Reaction (qRT-PCR). Real-time PCR was performed using QuantStudio 7 Flex Real-Time PCR System as described previously.^{43,44} RNA was purified using the Qiagen RNase-Free DNase set, then after quantification, the extracted RNA was converted to cDNA using High Capacity RNA-to-cDNA Kit from Applied Biosystems (Thermo Fisher Scientific). The levels of AR, TMPRSS2, FKBP5, PSA(KLK3) and GAPDH were quantified using TaqMan Fast Advanced Master Mix from Applied Biosystems. The level of gene expression was evaluated using comparative CT method, which compares the CT value to GAPDH (ACT) and then to vehicle control (ΔΔCT).

Cloning and Purification of VHL-ElonginBC complex. The DNA sequence of VHL (coding for residues 54-213) was constructed by PCR and inserted into a His-TEV expression vector⁴⁵ using ligation-independent cloning. The DNA sequences of Elongin B (encoding residues 1-118) and Elongin C (encoding residues 1-96) were constructed by PCR and inserted into pCDFDuet 1 using Gibson assembly⁴⁶. BL21(DE3) cells were transformed simultaneously with both plasmids and grown in Terrific Broth at 37° C. until an OD600 of 1.2. The cells were induced overnight with 0.4 mM IPTG at 24° C. Pelleted cells were freeze-thawed then resuspended in 20 mM Tris HCl pH 7.0, 200 mM NaCl and 0.1% β-mercaptoethanol (bME) containing protease inhibitors. The cell suspension was lysed by sonication and debris removed via centrifugation. The supernatant was incubated at 4° C. for 1 hr with N1-NTA (Qiagen) pre-washed in 20 mM Tris-HCl pH 7.0, 200 mM NaCl and 10 mM Imidazole. The protein complex was eluted in 20 mM Tris-HCl pH 7.0, 200 mM NaCl and 300 mM Imidazole, dialyzed into 20 mM Tris-HCl pH 7.0, 150 mM NaCl, and 0.01% bME and incubated with TEV protease overnight at 4° C. The protein sample was reapplied to the N1-NTA column to remove the His-tag. The flow through containing the VHL complex was diluted to 75 mM NaCl and applied to a HiTrap Q column (GE Healthcare). The sample was eluted with a salt gradient (0.075-1 M NaCl), concentrated and further purified on a Superdex S75 column (GE Healthcare) pre-equilibrated with 20 mM Bis-Tris 7.0, 150 mM NaCl and 1 mM DTT. Samples were aliquoted and stored at −80° C.

Binding Affinities of VHL Ligands to VHL-ElonginBC Complex Protein. The IC₅₀ and Kᵢ values of compounds were determined in competitive binding experiments. Mixtures of 5 μL of solutions of compounds in DMSO and 95 μL of preincubated protein/tracer complex solution were added into assay plates which were incubated at room temperature for 60 min with gentle shaking. The final concentrations of VHL protein and fluorescent probe were both 5 nM. Negative controls containing protein/probe complex only (equivalent to 0% inhibition) and positive controls containing only free probes (equivalent to 100% inhibition) were included in each assay plate. FP values in millipolarization units (mP) were measured using the Infinite M-1000 plate reader (Tecan U.S., Research Triangle Park, N.C.) in Microfluor 1 96-well, black, round-bottom plates (Thermo Scientific, Waltham, Mass.) at an excitation wavelength of 485 nm and an emission wavelength of 530 nm. IC₅₀ values were determined by nonlinear regression fitting of the competition curves. Kᵢ values of competitive inhibitors were obtained directly by nonlinear regression fitting, based upon the K_{D} values of the probe and concentrations of the protein and probe in the competitive assays. All the FP competitive experiments were performed in duplicate in three independent experiments.

Western Blotting. Treated cells were lysed by RIPA buffer supplemented with protease and phosphatase inhibitors. The cell lysates were separated by 4-12% SDS-PAGE gels and blotted into PVDF membranes. Software ImageJ was used to quantify the percentage of AR degradation. The net protein bands and loading controls are calculated by deducting the background from the inverted band value. The final relative quantification values are the ratio of net band to net loading control.

Pharmacodynamics Studies in the VCaP Xenograft Models in Mice. All animal experiments were performed under the guidelines of the University of Michigan Committee for Use and Care of Animals and using an approved animal protocol (PI, Shaomeng Wang). Xenograft tumors were established by injecting 5×106 VCaP cells in 50% Matrigel subcutaneously on the dorsal side of severe combined immunodeficient (SCID) mice, obtained from Charles River, one tumor per mouse. When tumors reached ˜100 mm³, mice were randomly assigned to treatment and vehicle control groups. Animals were monitored daily for any signs of toxicity and weighed 2-3 times per week during the treatment period and at least weekly after the treatment ended. For pharmacodynamics analysis, resected control and treated VCaP xenograft tumor tissues were ground into powder in liquid nitrogen and lysed in CST lysis buffer with halt proteinase inhibitors. Twenty micrograms of whole tumor clarified lysates were separated on 4-20% or 4-12% Novex gels. Western blots were performed as detailed in the previous section.

### Example 5

### Compounds of the Disclosure Degrade AR Protein in LNCaP Cells

Enzalutamide (4) was tethered to the terminal amide group in VHL ligands using various linkers to give compounds of Formula H and Formula I. The percent AR protein degradation induced by these compounds at various concentrations in LNCaP cells is shown in Table 1 and Table 2, respectively.

TABLE 1

| | |
|---|---|
| | II |
| 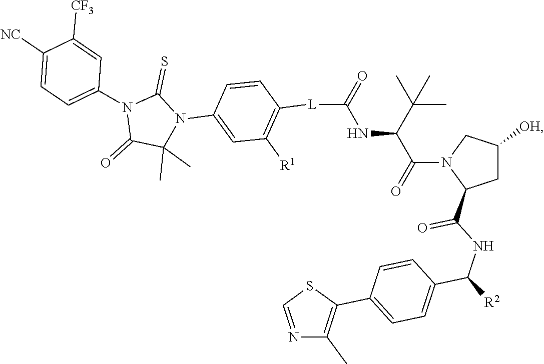 | |

| | | | | % AR protein degradation | | | |
|---|---|---|---|---|---|---|---|
| | L | | | in LNCaP Cells (μM) | | | |
| Compound | (Linker) | R¹ | R² | 0.01 | 0.1 | 1 | 10 |
| DMSO | — | | | 0 | 0 | 0 | 0 |
| 4 | — | | | — | — | 30 | 19 |
| 8 | 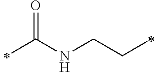 | F | H | — | 26 | 35 | 28 |
| 9 | 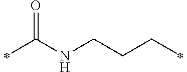 | F | H | — | 15 | 23 | 33 |
| 10 | 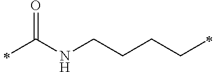 | F | H | — | 16 | 20 | 25 |
| 11 | 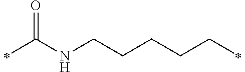 | F | H | — | 11 | 25 | 29 |
| 12 | 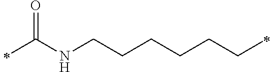 | F | H | — | 54 | 84 | 64 |
| 13 | 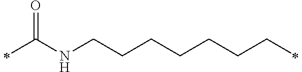 | F | H | 8 | 29 | 65 | — |
| 14 | 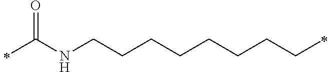 | F | H | 15 | 66 | 87 | — |
| 15 | 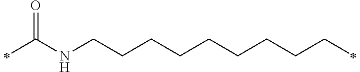 | F | H | 10 | 48 | 88 | — |
| 16 | 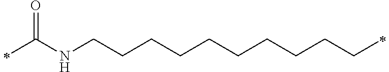 | F | H | 11 | 48 | 86 | — |
| 17 | 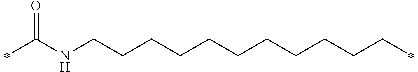 | F | H | — | 30 | 69 | 75 |
| 18 | 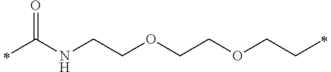 | F | H | — | 32 | 35 | 40 |
| 19 | 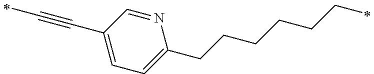 | H | H | 30 | 63 | 96 | — |
| 20 | 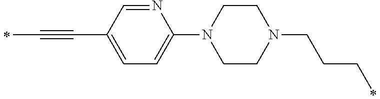 | H | H | — | 50 | 80 | 80 |
| 21 | 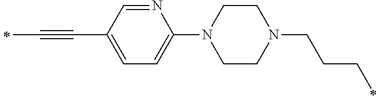 | H | Me | 37 | 61 | 92 | — |
| 22 | 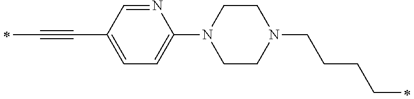 | H | Me | 28 | 48 | 89 | — |
| 23 | 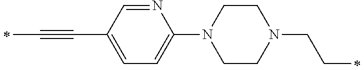 | H | Me | 34 | 51 | 78 | — |
| 24 | 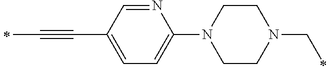 | H | Me | — | 3 | 31 | 28 |
| 25 | 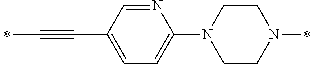 | H | Me | — | 0 | 12 | 0 |

All the data were average of three independent experiments with a treatment time of 6 h Table 2

TABLE 2

| | |
|---|---|
| | III |
| 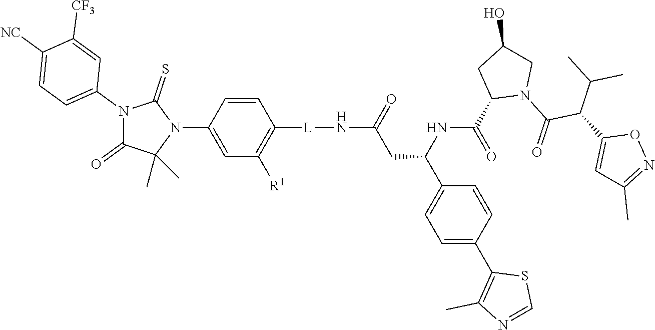 | |

| | | | % AR protein | | | |
|---|---|---|---|---|---|---|
| | | | degradation in LNCaP | | | |
| | L | | Cells (μM) | | | |
| Compound | (Linker) | R¹ | 0.01 | 0.1 | 1 | 10 |
| DMSO | — | | 0 | 0 | 0 | 0 |
| 4 | — | | — | — | 30 | 19 |
| 26 | 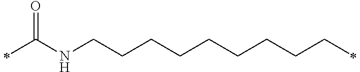 | F | 11 | 50 | 87 | — |
| 27 | 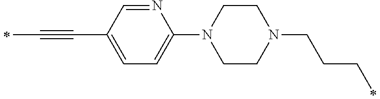 | H | 27 | 75 | 94 | — |
| 28 | 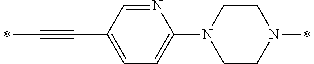 | H | 22 | 72 | 93 | — |
| 29 | 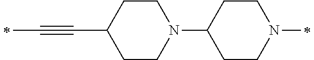 | H | 20 | 81 | 97 | — |

All the data were average of three independent experiments with a treatment time of 6 h

Various androgen receptor antagonists were tethered to a VHL ligand to give compounds of Formula IV. The percent AR protein degradation induced by these compounds at various concentrations in LNCaP cells is shown in Table 3.

TABLE 3

| | |
|---|---|
| | IV |
| 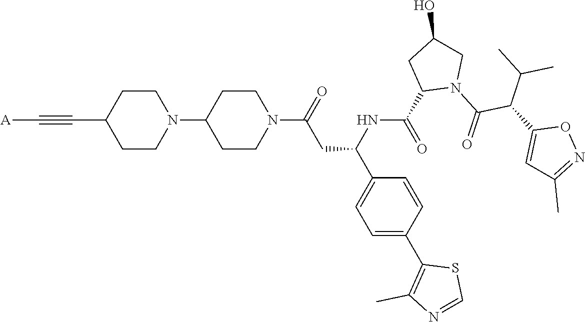 | |

| | | % AR protein degradation in | | | |
|---|---|---|---|---|---|
| | A | LNCaP Cells (μM) | | | |
| Compound | (AR antagonist) | 0.01 | 0.1 | 1 | 10 |
| DMSO | — | 0 | 0 | 0 | 0 |
| 29 | 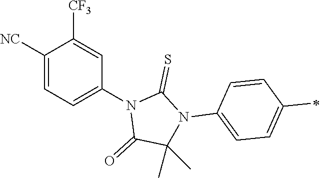 | 20 | 81 | 97 | — |
| 30 | 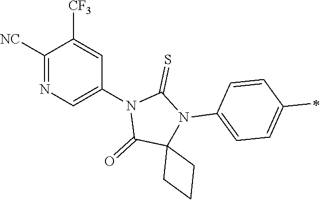 | 38 | 80 | 95 | — |
| 31 | 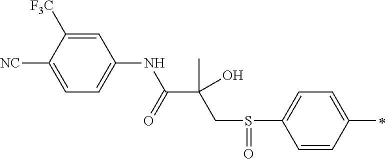 | — | 18 | 34 | 35 |
| 32 | 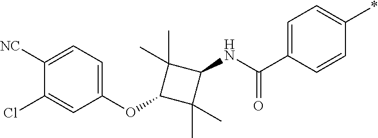 [SMILES: *c1ccc(C(=O)N[C@H]2C(C)(C)[C@H](Oc3ccc(C#N)c(Cl)c3)C2(C)C)cc1] | 76 | 98 | 99 | — |
| 33 | 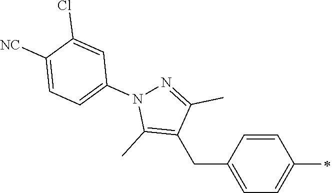 | — | 65 | 93 | 78 |

All the data were average of three independent experiments with a treatment time of 6 h

Various VHL ligands were tethered to androgen receptor antagonist to give compounds of Formula V. The percent AR protein degradation induced by these compounds at various concentrations in LNCaP cells is shown in Table 4.

TABLE 4

| | |
|---|---|
| | V |
| 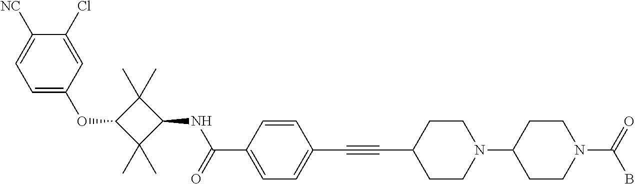 | |

| | | % AR protein degradation in | | | |
|---|---|---|---|---|---|
| | | LNCaP Cells (μM) | | | |
| Compound | B | 0.01 | 0.1 | 1 | 10 |
| DMSO | — | 0 | 0 | 0 | 0 |
| 6 | — | — | — | 24 | 47 |
| 32 | 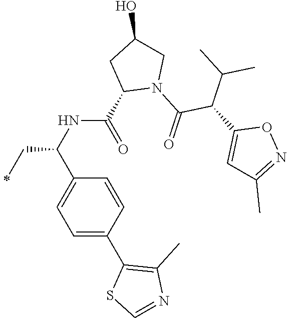 | 76 | 98 | 99 | — |
| 34 (ARD-69) | 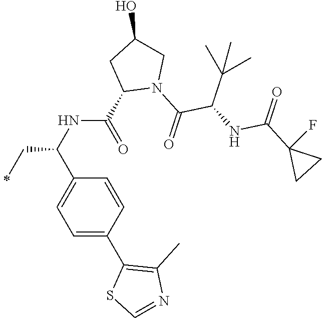 | 89 | 99 | 100 | — |
| 35 | 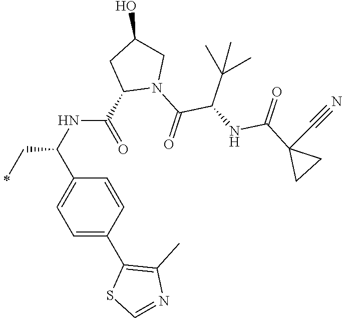 | 76 | 99 | 100 | — |
| 36 | 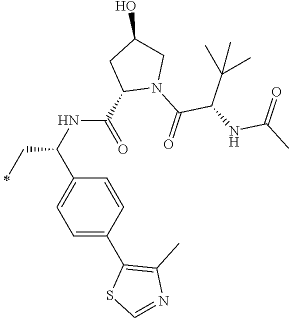 | 39 | 77 | 99 | — |
| 37 | 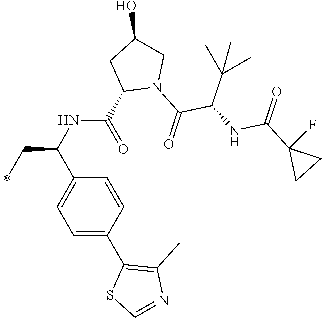 | — | <5 | <5 | <5 |
| 38 | 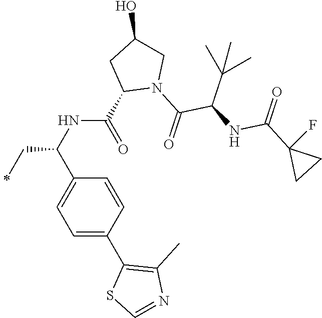 | — | <5 | <5 | <5 |

All the data were average of three independent experiments with a treatment time of 6 h

Various VHL ligands were tethered to an androgen receptor antagonist using to give compounds of Formula VI. The percent AR protein degradation induced by these compounds at various concentrations in LNCaP cells is shown in Table 5

TABLE 5

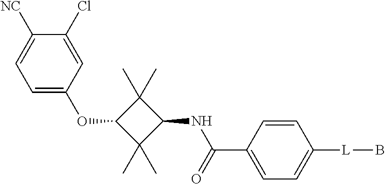

| | | | % of AR protein | | | |
|---|---|---|---|---|---|---|
| | | | degradation | | | |
| | L | B | in LNCaP Cells (μM) | | | |
| Compound | (Linker) | (E3 Ligand) | 0.01 | 0.1 | 1 | 10 |
| DMSO | — | | 0 | 0 | 0 | 0 |
| 39 32 | L1 L2 | 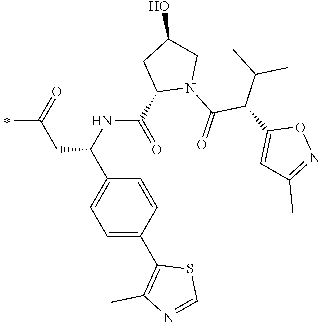 | 43 76 | 91 98 | 96 99 | — — |
| 40 41 | L1 L2 | 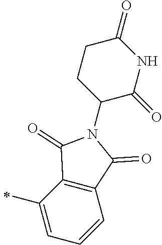 | — — | 7 2 | 20 25 | 5 20 |

L1 = 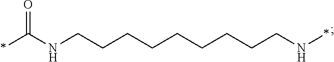 [SMILES: *NCCCCCCCCCNC(*)=O]
L2 =

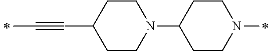

All the data were average of three independent experiments with a treatment time of 6 h

### Example 6

### AR Degradation Studies

Compounds 32, 34 and 35 were evaluated or their dose-dependent AR degradation in the LNCaP cell line, with compound 6 included as the AR antagonist control. Western blotting data showed that each of these three AR degraders induces AR degradation in a dose-dependent manner, whereas the AR antagonist (6) is completely ineffective, even at 10 μM. While compounds 32, 34 and 35 induce partial AR degradation at 10 nM, they all induce essentially complete AR degradation at both 100 nM and 1 μM. Based upon the data obtained at 10 nM, compound 34 (ARD-69 was the most potent AR degrader among 32, 34 and 35

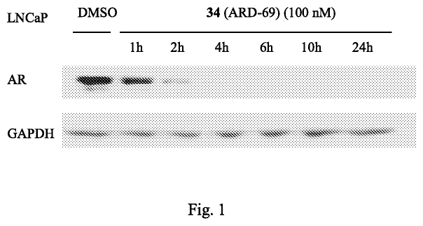

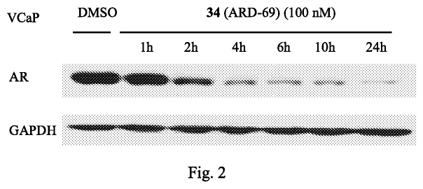

The kinetics of ARD-69 in induction of AR degradation at 100 nM in the LNCaP and VCaP AR+ cell lines was evaluated. The data showed that ARD-69 effectively reduced the AR protein level within 2 h and achieved near-complete AR depletion with a 4 h treatment indicating that induced AR degradation by ARD-69 in LNCaP and VCaP cells was rapid. See FIG. 1 and FIG. 2.

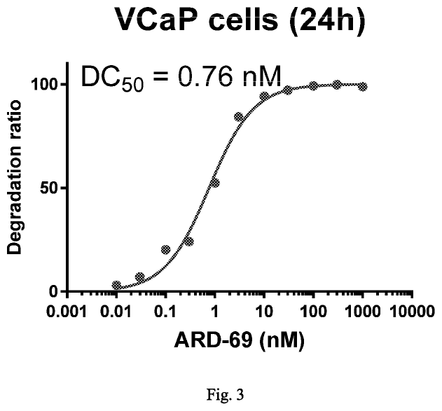

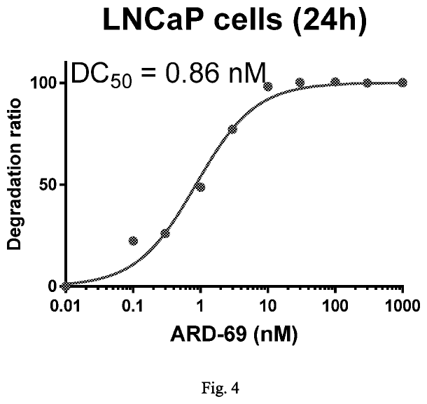

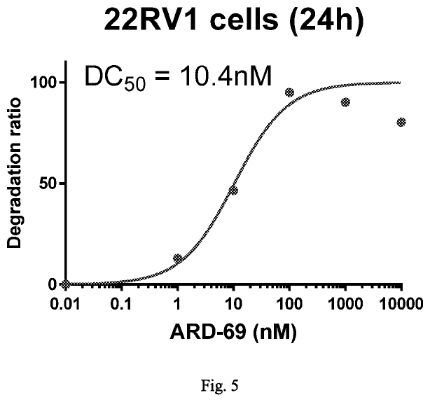

ARD-69 was evaluated in LNCaP, VCaP and 22RV1 cell lines for its potency in inducing AR degradation with a 24 h treatment time. ARD-69 achieves DC₅₀ (the drug concentration that results in 50% protein degradation) values of 0.86 nM and 0.76 nM in the LNCaP (FIG. 3) and VCaP (FIG. 4) cell lines and >95% AR degradation at 10 nM in both cell lines. ARD-69 achieves a DC₅₀ of 10.4 nM and near complete degradation at 1 μM in 22RV1 cells (FIG. 5).

The ability of ARD-69 to suppress AR-regulated gene expression in LNCaP and VCaP cell lines, with AR antagonist (6) included as the control, was investigated. The data showed that ARD-69 effectively suppressed the expression of PSA, TMPRSS2 and FKBP5 genes in both LNCaP and VCaP cell lines in a dose-dependent manner and is capable of reducing the mRNA level of both PSA and TMPRSS2 genes by >50% at 10 nM. In addition, ARD-69 suppresses the ERG gene in VCaP cell lines in a dose-dependent manner. In direct comparison, ARD-69 is >100-times more potent than AR antagonist 6 in suppressing the AR-regulated gene transcription in both LNCaP and VCaP cell lines. Data not shown.

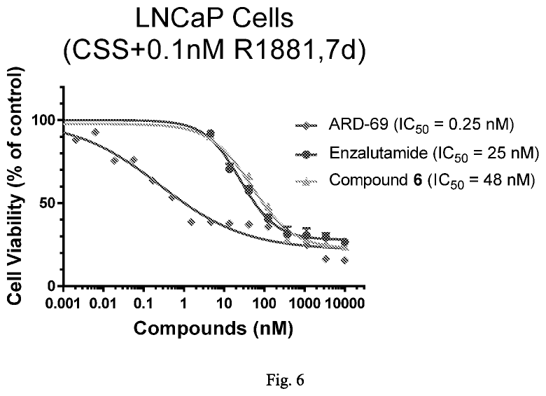

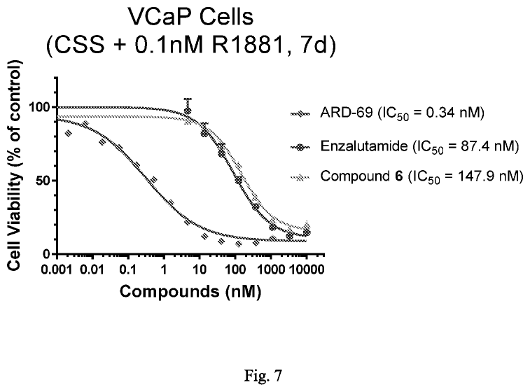

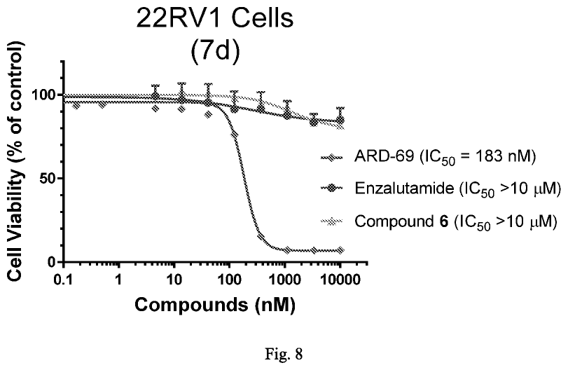

Because AR signaling drives cell growth for AR-positive prostate cancer cells, the ability of ARD-69 to inhibit cell growth, with enzalutamide (4) and compound 6 included as the AR antagonist controls, was tested. The data showed that ARD-69 is inhibits cell growth in both LNCaP and VCaP cell lines and achieves IC₅₀ values of 0.25 nM and 0.34 nM in the LNCaP (FIG. 6) and VCAP (FIG. 7) cell lines, respectively. In the 22RV1 cell line, compound 34 (ARD-69) achieves an IC₅₀ value of 183 nM and AR antagonists 4 and 6 have IC₅₀ values of >10 μM (FIG. 8). In sum, ARD-69 is >100-times more potent than enzalutamide (4) and compound 6 in LNCaP, VCaP and 22RV1 AR+ prostate cancer cell lines.

The mechanism of AR degradation induced by ARD-69 in LNCaP and VCAP cells was investigated. AR degradation induced by ARD-69 can be effectively blocked by pretreatment with an AR antagonist (6), a VHL ligand (VHL-d), a NEDD8 activating E1 enzyme inhibitor (MLN4924) or a proteasome inhibitor (MG132) in both LNCaP and VCAP cell lines (data not shown). These mechanistic data demonstrate that ARD-69 is a PROTAC AR degrader.

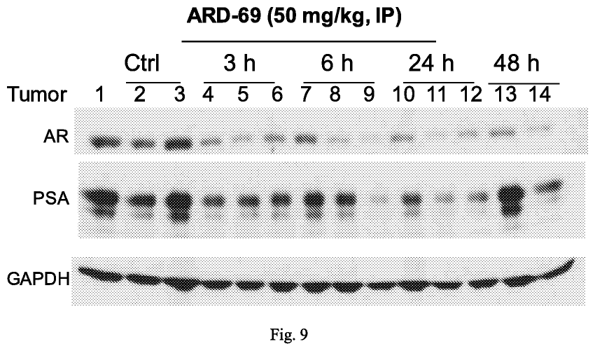

The pharmacodynamics (PD) of ARD-69 in the VCaP xenograft tumor tissue in mice was examined. The PD data showed that a single administration of ARD-69 at 50 mg/kg via intraperitoneal (IP) injection effectively reduced the level of AR protein, starting at 3 h and with the effect persisting for at least 48 h. Consistent with the profound decrease of the AR protein, the level of PSA protein was also effectively reduced at the 3 h time-point with the effect persisting for 24-48 h. See FIG. 9.

### Example 7

### Synthesis of VHL Ligands

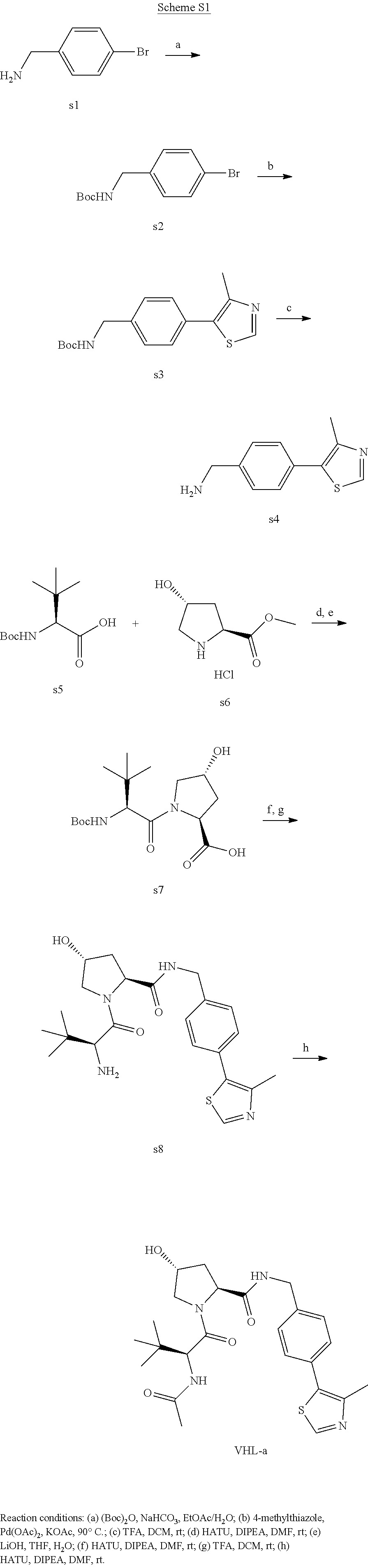

### General Procedure for Synthesis of Compounds VHL-a-b.

s1 (4-bromophenyl)methanamine (1.85 g, 10 mmol) was dissolved in EtOAc/H₂O (20 mL/20 mL) and then sodium bicarbonate (0.7 eq.) and Boc₂O (1.2 eq.) were added at stirring. The resulting reaction mixture was stirred for 1 h at room temperature. The aqueous layer was extracted with ethyl acetate and combined organic layers were washed with brine and dried over anhydrous Na₂SO₄. The solvent was removed on a rotary evaporator and the white residue s2 was used for the next step without further purification (95% yield).

s2 (2.7 g, 9.5 mmol), 4-methylthiazole (2 eq.), KOAc (2 eq.) and Pd(OAc)₂ (1%) were added to a round-bottom flask under Ar. DMF (30 mL) was added at room temperature. The solution was heated to 90° C. and stirred for 2 h. After UPLC-MS demonstrated the full conversion of starting materials, the reaction mixture was cooled to room temperature and water was added into the mixture. The aqueous layer was extracted with ethyl acetate, the combined organic layers were washed with brine, then dried over anhydrous Na₂SO₄. The solvent was removed on a rotary evaporator and the residue was purified by flash column chromatogram. The desired intermediate s3 was isolated in 85% yield.

A solution of s3 methyl tert-butyl (4-(4-methylthiazol-5-yl)benzyl)carbamate in 1:1 TFA:DCM was stirred at room temperature for 30 min. The solvents were evaporated under reduced pressure to give the corresponding deprotected intermediate s4 (4-(4-methylthiazol-5-yl)phenyl)methanamine (TFA salt) that was used in the following reactions without further purification (95% yield).

To a solution of the s5 (S)-2-((tert-butoxycarbonyl)amino)-3,3-dimethylbutanoic acid (2.32 g, 10 mmol) and S6 methyl (2S,4R)-4-hydroxypyrrolidine-2-carboxylate hydrogen chloride (1.1 equiv) in DMF was added DIPEA (6 equiv) and HATU (1.2 equiv). Then the mixture was stirred at r.t. overnight. The mixture was quenched with water and extracted with EA three times. The organic layer was washed with 5% (v/v) citric acid, NaHCO₃ solution, brine and dried with Na₂SO₄. The product was obtained by removing the solvent for the next step without further purification. Then, s7 can be obtained through the hydrolysis by LiOH in THF/H₂O (85% yield).

To a solution of the s7 (2S,4R)-1-((S)-2-((tert-butoxycarbonyl)amino)-3,3-dimethylbutanoyl)-4-hydroxypyrrolidine-2-carboxylic acid (2.92 g, 8.5 mmol) and s4 (1.1 equiv) in DMF was added DIPEA (6 equiv) and HATU (1.2 equiv). Then the mixture was stirred at r.t. overnight. The mixture was quenched with water and extracted with EA three times. The organic layer was washed with 5% (v/v) citric acid, NaHCO₃ solution, brine and dried with Na₂SO₄. The product was obtained by removing the solvent for the next step without further purification. Then, S8 can be obtained through the deprotection by TFA in DCM (88% yield).

To a solution of the s8 (2S,4R)-1-((S)-2-amino-3,3-dimethylbutanoyl)-4-hydroxy-N-(4-(4-methylthiazol-5-yl)benzyl)pyrrolidine-2-carboxamide (2.15 g, 5 mmol) and acetic acid (1.1 equiv) in DMF was added DIPEA (6 equiv) and HATU (1.2 equiv). Then the mixture was stirred at r.t. overnight. The mixture was quenched with water and extracted with EA three times. The organic layer was washed with 5% (v/v) citric acid, NaHCO₃ solution, brine and dried with Na₂SO₄. The product was obtained by removing the solvent and purified by flash column. The final product VHL-a can be obtained through removing the solvent as white solid (80% yield). Following the procedures used to prepare compound VHL-a, VHL-b was obtained with the same methods.

(2S,4R)-1-((S)-2-acetamido-3,3-dimethylbutanoyl)-4-hydroxy-N-(4-(4-methylthiazol-5-yl)benzyl)pyrrolidine-2-carboxamide (VHL-a). ¹H NMR (400 MHz, MeOD-d₄) δ 9.07 (s, 1H), 7.49 (d, J=4.1 Hz, 2H), 7.46-7.42 (m, 2H), 4.64 (s, 1H), 4.60-4.57 (m, 1H), 4.55-4.50 (m, 2H), 4.38 (d, J=15.5 Hz, 1H), 3.93 (dt, J=11.1, 1.8 Hz, 1H), 3.82 (dd, J=11.0, 3.9 Hz, 1H), 2.51 (s, 3H), 2.27-2.20 (m, 1H), 2.11 (td, J=8.9, 4.5 Hz, 1H), 2.02 (s, 3H), 1.05 (d, J=5.3 Hz, 9H). UPLC-MS calculated for C₂₄H₃₃N₄O₄S [M+H]⁺: 473.22, found: 473.25. UPLC-retention time: 2.8 min.

(2S,4R)-1-((S)-2-acetamido-3,3-dimethylbutanoyl)-4-hydroxy-N—((S)-1-(4-(4-methylthiazol-5-yl)phenyl)ethyl)pyrrolidine-2-carboxamide (VHL-b). ¹H NMR (400 MHz, MeOD-d₄) δ 9.39 (d, J=3.1 Hz, 1H), 7.53-7.46 (m, 4H), 4.66-4.57 (m, 2H), 4.49-4.42 (m, 1H), 3.91 (d, J=11.0 Hz, 1H), 3.76 (dd, J=11.1, 4.0 Hz, 1H), 2.55 (s, 3H), 2.27-2.17 (m, 1H), 2.02 (s, 3H), 2.02-1.94 (m, 2H), 1.53 (d, J=7.0 Hz, 3H), 1.07 (d, J=3.6 Hz, 9H). UPLC-MS calculated for C₂₅H₃₅N₄O₄S [M+H]⁺: 487.24, found: 487.21. UPLC-retention time: 3.0 min.

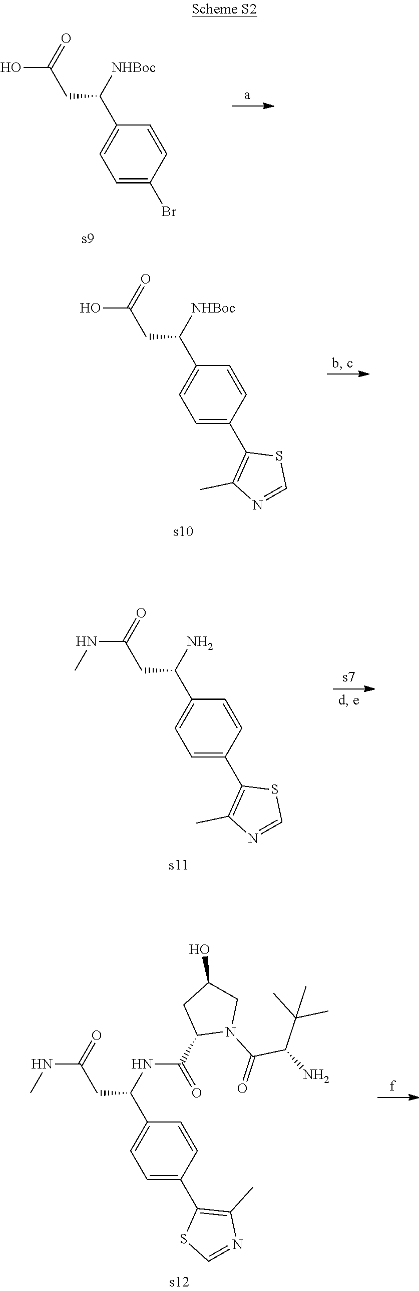 [SMILES: CC(C)(C)OC(=O)N[C@@H](CC(=O)O)c1ccc(Br)cc1 ; Cc1ncsc1-c1ccc([C@H](CC(=O)O)NC(=O)OC(C)(C)C)cc1 ; CNC(=O)C[C@H](N)c1ccc(-c2scnc2C)cc1 ; CNC(=O)C[C@H](NC(=O)[C@@H]1C[C@@H](O)CN1C(=O)[C@@H](N)C(C)(C)C)c1ccc(-c2scnc2C)cc1]

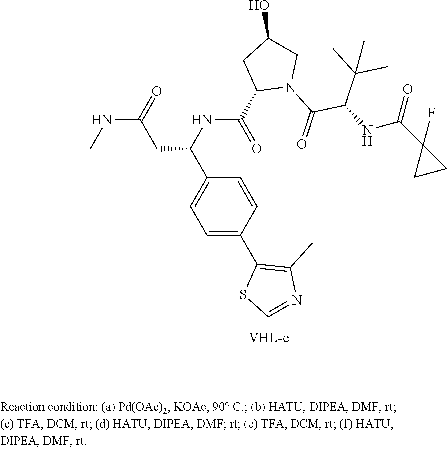 [SMILES: CNC(=O)C[C@H](NC(=O)[C@@H]1C[C@@H](O)CN1C(=O)[C@@H](NC(=O)C1(F)CC1)C(C)(C)C)c1ccc(-c2scnc2C)cc1]

### General Procedure for Synthesis of Compounds VHL-c-h.

A solution of s9 (S)-4-(4-bromophenyl)-4-((tert-butoxycarbonyl)amino)-2-oxobutanoic acid, 4-methylthiazole, KOAc and Pd(OAc)₂ in DMF/TEA was stirred at 80° C. for 4 h. After the reaction completed, TEA was removed under reduced pressure then added water into the mixture, the mixture was extracted by EA 3 times. The solvent was collected, dried with NaSO₄ and evaporated under reduced pressure to give the corresponding deprotected intermediate s10 (S)-4-((tert-butoxycarbonyl)amino)-4-(4-(4-methylthiazol-5-yl)phenyl)-2-oxobutanoic acid.

DIPEA (5 eq.) and HATU (1.2 eq.) were added to a solution of the s10 (72.4 mg, 0.2 mmol) and compound methylamine (1.1 eq.) in DMF (2 mL). After 30 min at room temperature, the mixture was subject to HPLC purification to afford compound s11 with 86% yield after deprotected by TFA/DCM.

DIPEA (5 eq.) and HATU (1.2 eq.) were added to a solution of the s11 (27.5 mg, 0.1 mmol) and compound s7 (1.1 eq.) in DMF (2 mL). After 30 min at room temperature, the mixture was subject to HPLC purification to afford compound s12 with 82% yield after deprotected by TFA/DCM.

DIPEA (5 eq.) and HATU (1.2 eq.) were added to a solution of the s12 (41 mg, 0.08 mmol) and compound fluorocyclopropane (1.1 eq.) in DMF (2 mL). After 30 min at room temperature, the mixture was subject to HPLC purification to afford compound s12 with 89% yield. Following the procedures used to prepare compound VHL-e, VHL-c, d, f, g and h were obtained with the same methods.

(2S,4R)-1-((S)-2-acetamido-3,3-dimethylbutanoyl)-4-hydroxy-N—((S)-3-(methylamino)-1-(4-(4-methylthiazol-5-yl)phenyl)-3-oxopropyl)pyrrolidine-2-carboxamide (VHL-c). ¹H NMR (400 MHz, MeOD-d₄) δ 9.33 (s, 1H), 7.50 (s, 4H), 5.33 (t, J=7.1 Hz, 1H), 4.63 (s, 1H), 4.59-4.53 (m, 1H), 4.46 (tt, J=4.2, 1.8 Hz, 1H), 3.90 (d, J=11.0 Hz, 1H), 3.78 (dd, J=11.0, 4.0 Hz, 1H), 2.89-2.74 (m, 2H), 2.68 (s, 3H), 2.54 (s, 3H), 2.25-2.13 (m, 1H), 2.02 (s, 3H), 1.96 (ddd, J=13.3, 9.0, 4.6 Hz, 1H), 1.06 (s, 9H). UPLC-MS calculated for C₂₇H₃₈N₅O₅S [M+H]⁺: 544.26, found: 544.24. UPLC-retention time: 2.0 min.

(2S,4R)-4-hydroxy-1-((R)-3-methyl-2-(3-methylisoxazol-5-yl)butanoyl)-N—((S)-3-(methylamino)-1-(4-(4-methylthiazol-5-yl)phenyl)-3-oxopropyl)pyrrolidine-2-carboxamide (VHL-d). ¹H NMR (400 MHz, MeOD-d₄) δ 9.02 (s, 1H), 7.47 (s, 4H), 6.23 (d, J=9.8 Hz, 1H), 5.40-5.29 (m, 1H), 4.56-4.42 (m, 2H), 3.89 (dd, J=10.8, 4.3 Hz, 1H), 3.80 (d, J=9.8 Hz, 1H), 3.65-3.57 (m, 1H), 2.84 (dt, J=10.0, 7.2 Hz, 1H), 2.79-2.72 (m, 1H), 2.68 (d, J=2.2 Hz, 3H), 2.51 (s, 3H), 2.47-2.39 (m, 1H), 2.27 (d, J=2.7 Hz, 3H), 2.21-2.14 (m, 1H), 2.03-1.94 (m, 1H), 1.41-1.30 (m, 1H), 1.08 (dd, J=6.5, 3.3 Hz, 3H), 0.90 (dd, J=12.5, 6.7 Hz, 3H). UPLC-MS calculated for C₂₈H₃₆N₅O₅S [M+H]⁺: 554.24, found: 554.23. UPLC-retention time: 2.5 min.

(2S,4R)-1-((S)-2-(1-fluorocyclopropane-1-carboxamido)-3,3-dimethylbutanoyl)-4-hydroxy-N—((S)-3-(methylamino)-1-(4-(4-methylthiazol-5-yl)phenyl)-3-oxopropyl)pyrrolidine-2-carboxamide (VHL-e). ¹H NMR (400 MHz, MeOD-d₄) δ 9.29 (s, 1H), 7.50 (s, 4H), 5.35 (t, J=7.1 Hz, 1H), 4.80-4.73 (m, 1H), 4.65-4.57 (m, 1H), 4.47 (tt, J=4.0, 1.8 Hz, 11H), 3.88-3.76 (m, 2H), 3.22 (t, J=7.3 Hz, 2H), 3.04-2.71 (m, 3H), 2.67 (d, J=13.6 Hz, 3H), 2.54 (s, 3H), 2.23 (ddt, J=13.2, 7.7, 1.8 Hz, 1H), 1.99 (ddt, J=13.4, 9.3, 4.6 Hz, 1H), 1.33 (dd, J=9.3, 5.4 Hz, 6H), 1.07 (d, J=5.3 Hz, 9H). UPLC-MS calculated for C₂₉H₃₉FN₅O₅S [M+H]⁺: 588.27, found: 588.28. UPLC-retention time: 2.8 min.

(2S,4R)-1-((S)-2-(1-cyanocyclopropane-1-carboxamido)-3,3-dimethylbutanoyl)-4-hydroxy-N—((S)-3-(methylamino)-1-(4-(4-methylthiazol-5-yl)phenyl)-3-oxopropyl)pyrrolidine-2-carboxamide (VHL-f). ¹H NMR (400 MHz, MeOD-d₄) δ 9.39 (s, 1H), 7.60-7.44 (m, 4H), 5.34 (t, J=7.1 Hz, 1H), 4.80-4.74 (m, 1H), 4.61-4.46 (m, 2H), 3.91-3.78 (m, 2H), 2.92-2.83 (m, 11H), 2.76 (dd, J=14.3, 7.7 Hz, 1H), 2.67 (d, J=12.5 Hz, 3H), 2.55 (s, 3H), 2.33-2.17 (m, 1H), 2.11-1.96 (m, 1H), 1.43-1.27 (m, 6H), 1.09 (d, J=2.9 Hz, 9H). UPLC-MS calculated for C₃₀H₃₉N₆O₅S [M+H]⁺: 595.27, found: 595.25. UPLC-retention time: 1.6 min.

(2S,4R)-1-((S)-2-(1-fluorocyclopropane-1-carboxamido)-3,3-dimethylbutanoyl)-4-hydroxy-N—((R)-3-(methylamino)-1-(4-(4-methylthiazol-5-yl)phenyl)-3-oxopropyl)pyrrolidine-2-carboxamide (VHL-g). ¹H NMR (400 MHz, MeOD-d₄) δ 9.27 (s, 1H), 7.50 (s, 4H), 5.39-5.31 (m, 1H), 4.78-4.74 (m, 1H), 4.64-4.57 (m, 1H), 4.47 (pd, J=4.0, 2.6, 1.8 Hz, 1H), 3.89-3.76 (m, 2H), 3.23 (q, J=7.3 Hz, 3H), 2.87 (dd, J=14.4, 6.7 Hz, 11H), 2.78 (q, J=7.2 Hz, 1H), 2.67 (d, J=13.6 Hz, 3H), 2.54 (s, 3H), 2.23 (ddt, J=13.2, 7.6, 1.8 Hz, 1H), 1.98 (ddd, J=13.4, 9.3, 4.4 Hz, 1H), 1.38-1.31 (m, 6H), 1.07 (d, J=5.3 Hz, 9H). UPLC-MS calculated for C₂₉H₃₉FN₅O₅S [M+H]⁺: 588.27, found: 588.28. UPLC-retention time: 2.8 min.

(2S,4R)-1-((R)-2-(1-fluorocyclopropane-1-carboxamido)-3,3-dimethylbutanoyl)-4-hydroxy-N—((S)-3-(methylamino)-1-(4-(4-methylthiazol-5-yl)phenyl)-3-oxopropyl)pyrrolidine-2-carboxamide (VHL-h). ¹H NMR (400 MHz, MeOD-d₄) δ 9.20 (d, J=3.2 Hz, 1H), 7.49 (d, J=3.3 Hz, 4H), 5.41-5.30 (m, 1H), 4.76 (d, J=6.3 Hz, 1H), 4.61 (dq, J=12.0, 4.2, 3.6 Hz, 1H), 4.51-4.44 (m, 1H), 3.90-3.76 (m, 2H), 3.23 (q, J=7.2 Hz, 3H), 2.93-2.82 (m, 1H), 2.78 (dt, J=9.9, 5.8 Hz, 1H), 2.69 (d, J=3.3 Hz, 3H), 2.53 (d, J=3.2 Hz, 3H), 2.23 (ddt, J=13.2, 7.4, 1.9 Hz, 1H), 1.98 (ddd, J=13.3, 9.3, 4.4 Hz, 1H), 1.33 (td, J=7.4, 3.2 Hz, 6H), 1.08 (d, J=3.4 Hz, 10H). UPLC-MS calculated for C₂₉H₃₉FN₅O₅S [M+H]⁺: 588.27, found: 588.28. UPLC-retention time: 2.8 min.

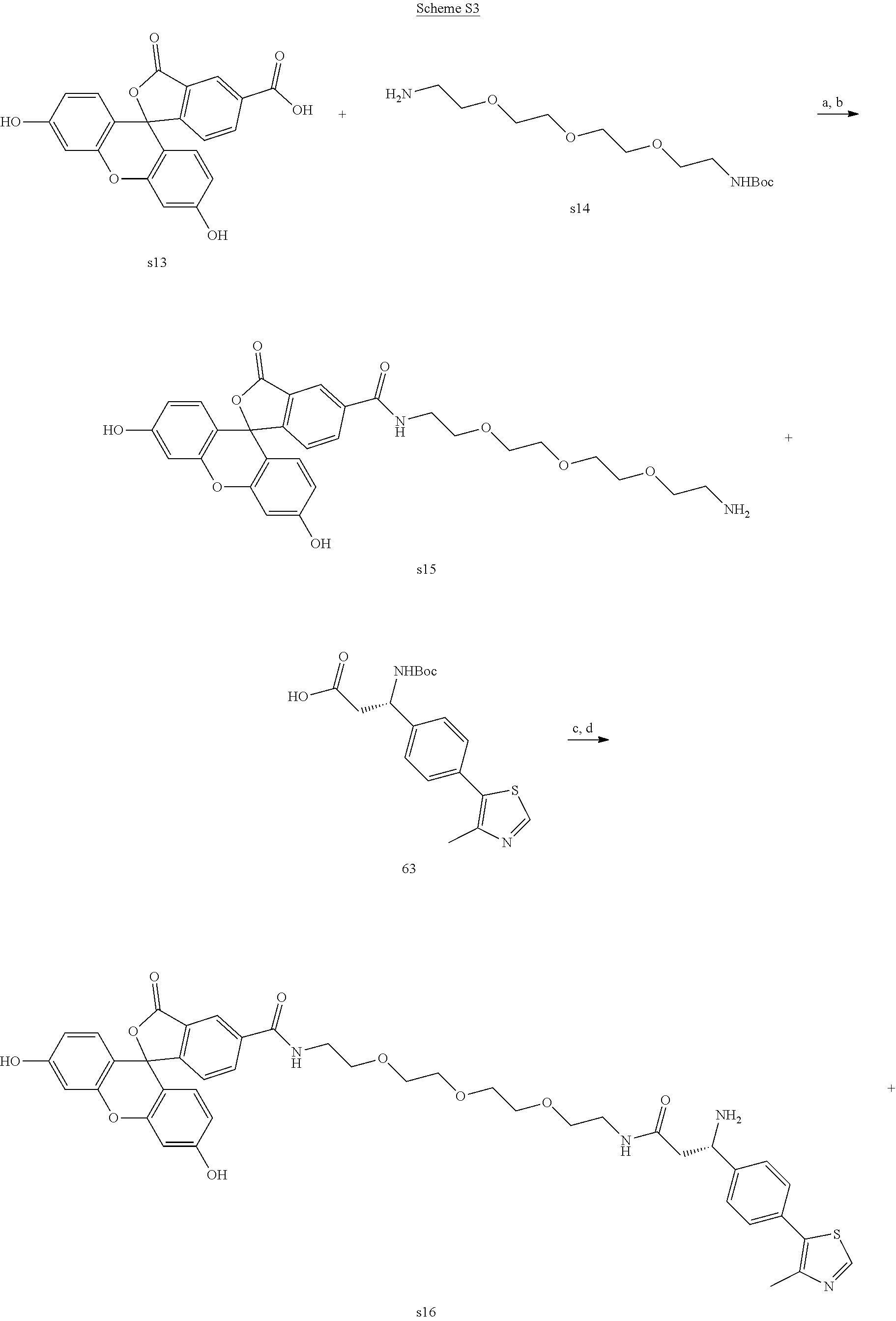 [SMILES: O=C(O)c1ccc2c(c1)C(=O)OC21c2ccc(O)cc2Oc2cc(O)ccc21 ; CC(C)(C)OC(=O)NCCOCCOCCOCCN ; NCCOCCOCCOCCNC(=O)c1ccc2c(c1)C(=O)OC21c2ccc(O)cc2Oc2cc(O)ccc21 ; Cc1ncsc1-c1ccc([C@H](CC(=O)O)NC(=O)OC(C)(C)C)cc1 ; Cc1ncsc1-c1ccc([C@@H](N)CC(=O)NCCOCCOCCOCCNC(=O)c2ccc3c(c2)C(=O)OC32c3ccc(O)cc3Oc3cc(O)ccc32)cc1]

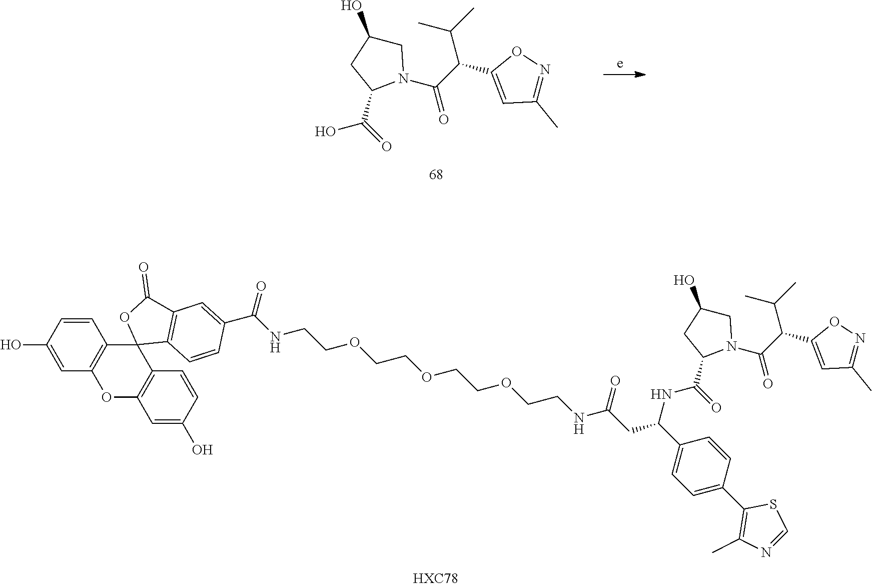 [SMILES: Cc1cc([C@H](C(=O)N2C[C@H](O)C[C@H]2C(=O)O)C(C)C)on1 ; Cc1cc([C@H](C(=O)N2C[C@H](O)C[C@H]2C(=O)N[C@@H](CC(=O)NCCOCCOCCOCCNC(=O)c2ccc3c(c2)C(=O)OC32c3ccc(O)cc3Oc3cc(O)ccc32)c2ccc(-c3scnc3C)cc2)C(C)C)on1]

### General Procedure for Synthesis of Compound HXC78.

DIPEA (5 eq.) and HATU (1.2 eq.) were added to a solution of the s13 (75.2 mg, 0.2 mmol) and compound s14 (1.1 eq.) in DMF (2 mL). After 30 min at room temperature, the mixture was subject to HPLC purification to afford compound s15 with 84% yield after deprotected by TFA/DCM.

DIPEA (5 eq.) and HATU (1.2 eq.) were added to a solution of the s15 (55 mg, 0.1 mmol) and compound 63 (1.1 eq.) in DMF (2 mL). After 30 min at room temperature, the mixture was subject to HPLC purification to afford compound s16 with 88% yield after deprotected by TFA/DCM.

DIPEA (5 eq.) and HATU (1.2 eq.) were added to a solution of the s16 (40 mg, 0.05 mmol) and compound 68 (1.1 eq.) in DMF (2 mL). After 30 min at room temperature, the mixture was subject to HPLC purification to afford compound HXC78 with 90% yield.

(2S,4R)—N—((S)-1-(3′,6′-dihydroxy-3-oxo-3H-spiro[isobenzofuran-1,9′-xanthen]-5-yl)-17-(4-(4-methylthiazol-5-yl)phenyl)-1,15-dioxo-5,8,11-trioxa-2,14-diazaheptadecan-17-yl)-4-hydroxy-1-((R)-3-methyl-2-(3-methylisoxazol-5-yl)butanoyl)pyrrolidine-2-carboxamide (HXC78). ¹H NMR (400 MHz, MeOD-d₄) δ 9.34 (s, 1H), 8.72-8.65 (m, 1H), 8.28 (dt, J=8.3, 2.6 Hz, 1H), 7.49 (q, J=12.7, 10.3 Hz, 5H), 7.10 (s, 3H), 6.93 (d, J=8.8 Hz, 2H), 6.21 (d, J=5.9 Hz, 11H), 5.36 (dd, J=8.0, 6.2 Hz, 2H), 4.49 (dd, J=18.4, 10.4 Hz, 2H), 3.88 (dd, J=10.8, 4.2 Hz, 1H), 3.86-3.51 (m, 14H), 3.44 (dt, J=9.6, 5.1 Hz, 2H), 3.24-3.01 (m, 3H), 2.91-2.71 (m, 3H), 2.53 (d, J=1.5 Hz, 3H), 2.41 (dt, J=9.8, 6.6 Hz, 1H), 2.25 (d, J=5.0 Hz, 3H), 1.97 (ddd, J=13.1, 8.6, 4.7 Hz, 1H), 1.41-1.27 (m, 2H), 1.11-0.98 (m, 3H), 0.88 (dd, J=12.8, 6.6 Hz, 3H). UPLC-MS calculated for C₅₆H₆₁N₆O₁₄S [M+H]⁺: 1073.40, found: 1073.41. UPLC-retention time: 3.4 min.

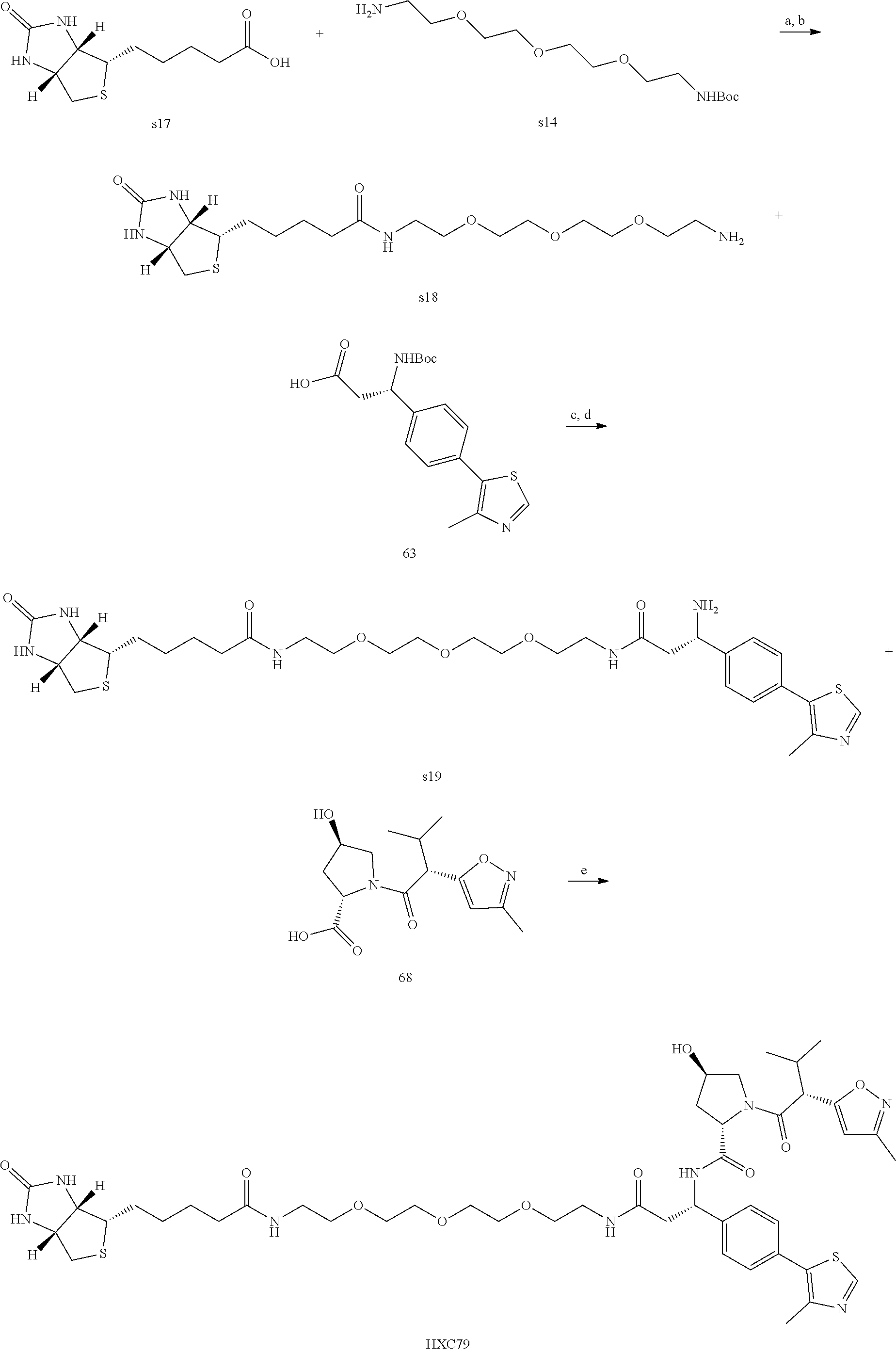

### General Procedure for Synthesis of Compounds HXC79

DIPEA (5 eq.) and HATU (1.2 eq.) were added to a solution of the s17 (48.8 mg, 0.2 mmol) and compound s14 (1.1 eq.) in DMF (2 mL). After 30 min at room temperature, the mixture was subject to HPLC purification to afford compound s18 with 87% yield after deprotected by TFA/DCM.

DIPEA (5 eq.) and HATU (1.2 eq.) were added to a solution of the s18 (41.8 mg, 0.1 mmol) and compound 63 (1.1 eq.) in DMF (2 mL). After 30 min at room temperature, the mixture was subject to HPLC purification to afford compound s19 with 82% yield after deprotected by TFA/DCM.

DIPEA (5 eq.) and HATU (1.2 eq.) were added to a solution of the s19 (33 mg, 0.05 mmol) and compound 68 (1.1 eq.) in DMF (2 mL). After 30 min at room temperature, the mixture was subject to HPLC purification to afford compound HXC79 with 88% yield.

(2S,4R)-4-hydroxy-1-((R)-3-methyl-2-(3-methylisoxazol-5-yl)butanoyl)-N—((S)-1-(4-(4-methylthiazol-5-yl)phenyl)-3,17-dioxo-21-((3aS,4S,6aR)-2-oxohexahydro-1H-thieno[3,4-d]imidazol-4-yl)-7,10,13-trioxa-4,16-diazahenicosyl)pyrrolidine-2-carboxamide (HXC79). ¹H NMR (400 MHz, MeOD-d₄) δ 9.19 (s, 1H), 7.55-7.46 (m, 4H), 6.23 (d, J=10.6 Hz, 1H), 5.37 (ddd, J=10.4, 6.4, 3.7 Hz, 1H), 4.56-4.45 (m, 3H), 4.32 (dd, J=7.9, 4.4 Hz, 1H), 3.93-3.76 (m, 2H), 3.62 (s, 6H), 3.57-3.52 (m, 4H), 3.45 (d, J=5.0 Hz, 1H), 3.37 (t, J=7.2 Hz, 3H), 3.26-3.10 (m, 2H), 3.03-2.76 (m, 4H), 2.72 (d, J=12.7 Hz, 1H), 2.42 (ddd, J=19.7, 10.2, 5.6 Hz, 2H), 2.32-2.24 (m, 3H), 2.25-2.17 (m, 3H), 2.12-1.91 (m, 3H), 1.68 (tdt, J=25.6, 13.3, 5.9 Hz, 5H), 1.51-1.34 (m, 3H), 1.08-0.88 (m, 6H). UPLC-MS calculated for C₄₅H₆₅N₈O₁₀S₂ [M+H]⁺: 941.43, found: 941.45. UPLC-retention time: 2.7 min.

Protein: The VHL complex is a multi-subunit ubiquitin ligase composed of VHL, Elongin B and Elongin C. Tracer: HXC78, Kd=2.4 nM f 0.3 nM.

The binding affinities of the VHL ligands are shown in Table Si.

TABLE S1

| ligand | VHL-a | VHL-b | VHL-c | VHL-d | VHL-e | VHL-f | VHL-g | VHL-h |
|---|---|---|---|---|---|---|---|---|
| IC₅₀ ± SD | 458 ± 33 | 164 ± 13 | 130 ± 5 | 26.8 ± 1.1 | 190 ± 36 | 28.6 ± 1.1 | 179 ± 37 | 78 ± 6 |
| (nM) | | | | | | | (μM) | (μM) |

### REFERENCES

(1) Hamdy, F. C.; Donovan, J. L.; Lane, J. A.; Mason, M.; Metcalfe, C.; Holding, P.; Davis, M.; Peters, T. J.; Turner, E. L.; Martin, R. M.; Oxley, J.; Robinson, M.; Staffurth, J.; Walsh, E.; Bollina, P.; Catto, J.; Doble, A.; Doherty, A.; Gillatt, D.; Kockelbergh, R.; Kynaston, H.; Paul, A.; Powell, P.; Prescott, S.; Rosario, D. J.; Rowe, E.; Neal, D. E. 10-Year Outcomes after Monitoring, Surgery, or Radiotherapy for Localized Prostate Cancer. N Engl J Med, 2016, 375, 1415-1424.
(2) Litwin, M. S.; Tan, H. J. The Diagnosis and Treatment of Prostate Cancer. JAMA, 2017, 317, 2532-2542.
(3) Karantanos, T.; Corn, P. G.; Thompson, T. C. Prostate cancer progression after androgen deprivation therapy: mechanisms of castrate resistance and novel therapeutic approaches. Oncogene. 2013, 32, 5501-511.
(4) Harris, W. P.; Mostaghel, E. A.; Nelson, P. S.; Montgomery, B. Androgen deprivation therapy: progress in understanding mechanisms of resistance and optimizing androgen depletion. Nat Clin Pract Urol, 2009, 6, 76-85.
(5) Narayanan, R.; Ponnusamy, S.; Miller, D. D. Destroying the androgen receptor (AR)-potential strategy to treat advanced prostate cancer. Oncoscience. 2017, 4, 175-177.
(6) Crowder, C. M.; Lassiter, C. S.; Gorelick, D. A. Nuclear Androgen Receptor Regulates Testes Organization and Oocyte Maturation in Zebrafish. Endocrinology. 2018, 159, 980-993.
(7) Sundén, H.; Holland, M. C.; Poutiainen, P. K.; Jssskeliinen, T.; Pulkkinen, J. T.; Palvimo, J. J.; Olsson, R. Synthesis and Biological Evaluation of Second-Generation Tropanol-Based Androgen Receptor Modulators. J. Med. Chem. 2015, 58, 1569-1574.
(8) Oksala, R.; Moilanen, A.; Riikonen, R.; Rummakko, P.; Karjalainen, A.; Passiniemi, M.; Wohlfahrt, G.; Taavitsainen, P.; Malmström, C.; Ramela, M.; Metsinkyli, H. M.; Huhtaniemi, R.; Kallio, P. J.; Mustonen, M. V. Discovery and Development of ODM-204: A Novel Nonsteroidal Compound for the Treatment of Castration-Resistant Prostate Cancer by blocking the Androgen Receptor and Inhibiting CYP17A1. J Steroid Biochem Mol Biol. 2018, doi: 10.1016/j.jsbmb.2018.02.004.
(9) Watson, P. A.; Arora, V. K.; Sawyers, C. L. Emerging mechanisms of resistance to androgen receptor inhibitors in prostate cancer. Nat Rev Cancer. 2015, 15, 701-711.
(10) Guo, C.; Linton, A.; Kephart, S.; Ornelas, M.; Pairish, M.; Gonzalez, J.; Greasley, S.; Nagata, A.; Burke, B. J.; Edwards, M.; Hosea, N.; Kang, P.; Hu, W.; Engebretsen, J.; Briere, D.; Shi, M.; Gukasyan, H.; Richardson, P.; Dack, K.; Underwood, T.; Johnson, P.; Morell, A.; Felstead, R.; Kuruma, H.; Matsimoto, H.; Zoubeidi, A.; Gleave, M.; Los, G.; Fanjul, A. N. Discovery of Aryloxy Tetramethylcyclobutanes as Novel Androgen Receptor Antagonists. J. Med. Chem. 2011, 54, 7693-7704.
(11) Moilanen, A. M.; Riikonen, R.; Oksala, R.; Ravanti, L.; Aho, E.; Wohlfahrt, G.; Nykänen, P. S.; Törmäkangas, O. P.; Palvimo, J. J.; Kallio, P. J. Discovery of ODM-201, a new generation androgen receptor inhibitor targeting resistance mechanisms to androgen signaling-directed prostate cancer therapies. Sci Rep. 2015, 5, 12007.
(12) Guerrini, A.; Tesei, A.; Ferroni, C.; Paganelli, G.; Zamagni, A.; Carloni, S. D.; Donato, M.; Castoria, G.; Leonetti, C.; Porru, M. D.; Cesare, M.; Zaffaroni, N.; Beretta, G. L. D; Rio, A.; Varchi, G. A New Avenue toward Androgen Receptor Pan-antagonists: C2 Sterically Hindered Substitution of Hydroxy-propanamides. J. Med. Chem. 2014, 57, 7263-7279.
(13) Jung, M. E.; Ouk, S.; Yoo, D.; Sawyers, C. L.; Chen, C.; Tran, C.; Wongvipat, J. Structure-activity relationship for thiohydantoin androgen receptor antagonists for castration-resistant prostate cancer (CRPC). J. Med. Chem. 2010, 53, 2779-2796.
(14) Yamamoto, S.; Tomita, N.; Suzuki, Y.; Suzaki, T.; Kaku, T.; Hara, T.; Yamaoka, M.; Kanzaki. N.; Hasuoka, A.; Baba, A.; Ito, M. Design, synthesis, and biological evaluation of 4-arylmethyl-1-phenylpyrazole and 4-aryloxy-1-phenylpyrazole derivatives as novel androgen receptor antagonists. Bioorg Med Chem. 2012, 20, 2338-2352.
(15) Balbas, M. D.; Evans, M. J.; Hosfield, D. J.; Wongvipat, J.; Arora, V. K.; Watson, P. A.; Chen, Y.; Greene, G. L.; Shen, Y.; Sawyers, C. L. Overcoming mutation-based resistance to antiandrogens with rational drug design. Elife. 2013, 2, e00499.
(16) Lottrup, G.; Jørgensen, A.; Nielsen, J. E.; Jørgensen, N.; Duno, M.; Vinggaard, A. M.; Skakkebæk, N. E.; Rajpert-De Meyts, E. Identification of a novel androgen receptor mutation in a family with multiple components compatible with the testicular dysgenesis syndrome. J Clin Endocrinol Metab. 2013, 98, 2223-2229.
(17) Zhu, S.; Zhao, D.; Yan, L.; Jiang, W.; Kim, J. S.; Gu, B.; Liu, Q.; Wang, R.; Xia, B.; Zhao, J. C.; Song, G.; Mi, W.; Wang, R. F.; Shi, X.; Lam, H. M.; Dong, X.; Yu, J.; Chen, K.; Cao, Q. BMI1 regulates androgen receptor in prostate cancer independently of the polycomb repressive complex 1. Nat Commun. 2018, 9, 500.
(18) Munuganti, R. S.; Hassona, M. D.; Leblanc, E.; Frewin, K.; Singh, K.; Ma, D.; Ban, F.; Hsing, M.; Adomat, H.; Lallous, N.; Andre, C.; Jonadass, J. P.; Zoubeidi, A.; Young, R. N.; Guns, E. T.; Rennie, P. S.; Cherkasov, A. Identification of a potent antiandrogen that targets the BF3 site of the androgen receptor and inhibits enzalutamide-resistant prostate cancer. Chem Biol. 2014, 21, 1476-485.
(19) Raina, K.; Lu, J.; Qian, Y.; Altieri, M.; Gordon, D.; Rossi, A. M.; Wang, J.; Chen, X.; Dong, H.; Siu. K.; Winkler, J. D.; Crew, A. P.; Crews, C. M.; Coleman, K. G. PROTAC-induced BET protein degradation as a therapy for castration-resistant prostate cancer. Proc Natl Acad Sci USA. 2016, 113, 7124-7129.
(20) Zhou, B.; Hu, J.; Xu, F.; Chen, Z.; Bai, L.; Fernandez-Salas, E.; Lin, M.; Liu, L.; Yang, C. Y.; Zhao. Y.; McEachern, D.; Przybranowski, S.; Wen, B.; Sun, D.; Wang, S. Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481.
(21) Gadd, M. S.; Testa, A.; Lucas, X.; Chan, K. H.; Chen, W.; Lamont, D. J.; Zengerle, M.; Ciulli, A. Structural basis of PROTAC cooperative recognition for selective protein degradation. Nat Chem. Biol. 2017, 13, 514-521.
(22) Toure, M.; Crews, C. M. Small-molecule PROTACS: new approaches to protein degradation. Angew. Chem. Int. Edn. 2016, 55, 1966-1973.
(23) Qin, C.; Hu, Y.; Zhou, B.; Fernandez-Salas, E.; Yang, C. Y.; Liu, L.; McEachern, D.; Przybranowski, S.; Wang, M.; Stuckey, J.; Meagher, J.; Bai, L.; Chen, Z.; Lin, M.; Yang, J.; Ziazadeh, D. N.; Xu, F.; Hu, J.; Xiang, W.; Huang, L.; Li, S.; Wen, B.; Sun, D.; Wang, S. Discovery of QCA570 as an Exceptionally Potent and Efficacious Proteolysis Targeting Chimera (PROTAC) Degrader of the Bromodomain and Extra-Terminal (BET) Proteins Capable of Inducing Complete and Durable Tumor Regression. J. Med. Chem. 2018, 61, 6685-6704.
(24) Hatcher, J. M.; Wang, E. S.; Johannessen, L.; Kwiatkowski, N.; Sim, T.; Gray, N. S. Development of Highly Potent and Selective Steroidal Inhibitors and Degraders of CDK8. ACS Med. Chem. Lett. 2018, 9, 540-545.
(25) Gollavilli, P. N.; Pawar, A.; Wilder-Romans, K.; Natesan, R.; Engelke, C. G.; Dommeti, V. L.; Krishnamurthy, P. M.; Nallasivam, A.; Apel, I. J.; Xu, T.; Qin, Z. S.; Feng, F. Y.; Asangani, I. A. EWS/ETS-Driven Ewing Sarcoma Requires BET Bromodomain Proteins. Cancer Res. 2018, 78, 4760-4773.
(26) Bondeson, D. P.; Crews, C. M. Targeted Protein Degradation by Small Molecules. Annu Rev Pharmacol Toxicol. 2017, 57, 107-123.
(27) Salami, J.; Alabi, S.; Willard, R. R.; Vitale, N. J.; Wang, J.; Dong, H.; Jin, M.; McDonnell, D. P.; Crew, A. P.; Neklesa, T. K.; Crews, C. M. Androgen receptor degradation by the proteolysis-targeting chimera ARCC-4 outperforms enzalutamide in cellular models of prostate cancer drug resistance. Commun Biol. 2018, 1, 100.
(28) Pal, S. K.; Patel, J.; He, M.; Foulk, B.; Kraft, K.; Smirnov, D. A.; Twardowski, P.; Kortylewski, M.; Bhargava, V.; Jones, J. O. Identification of mechanisms of resistance to treatment with abiraterone acetate or enzalutamide in patients with castration-resistant prostate cancer (CRPC). Cancer. 2018, 124, 1216-1224.
(29) Wang, C.; Peng, G.; Huang, H.; Liu, F.; Kong, D. P.; Dong, K. Q.; Dai, L. H.; Zhou, Z.; Wang, K. J.; Yang, J.; Cheng, Y. Q.; Gao, X.; Qu, M.; Wang, H. R.; Zhu, F.; Tian, Q. Q.; Liu, D.; Cao, L.; Cui, X. G.; Xu, C. L.; Xu, D. F.; Sun, Y. H. Blocking the Feedback Loop between Neuroendocrine Differentiation and Macrophages Improves the Therapeutic Effects of Enzalutamide (MDV3100) on Prostate Cancer. Clin Cancer Res. 2018, 24, 708-723.
(30) Gustafson, J. L.; Neklesa, T. K.; Cox, C. S.; Roth, A. G.; Buckley, D. L.; Tae, H. S.; Sundberg, T. B.; Stagg, D. B.; Hines, J.; McDonnell, D. P.; Norris, J. D.; Crews, C. M. Small-Molecule-Mediated Degradation of the Androgen Receptor through Hydrophobic Tagging. Angew. Chem. Int. Ed. 2015, 54, 9659-9662.
(31) Shibata, N.; Nagai, K.; Morita, Y.; Ujikawa, O.; Ohoka, N.; Hattori, T.; Koyama, R.; Sano, O.; Imaeda, Y.; Nara, H.; Cho, N.; Naito, M. Development of Protein Degradation Inducers of Androgen Receptor by Conjugation of Androgen Receptor Ligands and Inhibitor of Apoptosis Protein Ligands. J. Med. Chem. 2018, 61, 543-575.
(32) Crew, A. P.; Dong, H. Q.; Wang, J.; Ferraro, C.; Chen, X.; Qian, Y. M. U.S. Pat. Appl. Publ. 2017, US 20170327469 A120171116.
(33) Pereira de Jésus-Tran, K.; Côté, P. L.; Cantin, L.; Blanchet, J.; Labrie, F.; Breton, R. Comparison of crystal structures of human androgen receptor ligand-binding domain complexed with various agonists reveals molecular determinants responsible for binding affinity. Protein Sci. 2006, 15, 987-999.
(34) Galdeano, C.; Gadd, M. S.; Soares, P.; Scaffidi, S.; Van Molle, I.; Birced, I.; Hewitt, S.; Dias, D. M.; Ciulli, A. Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J. Med. Chem. 2014, 57, 8657-8663.
(35) Soares, P.; Gadd, M. S.; Frost, J.; Galdeano, C.; Ellis, L.; Epemolu, O.; Rocha, S.; Read, K. D.; Ciulli, A. Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J. Med. Chem. 2018, 61, 599-618.
(36) Buckley, D. L.; Van Molle, I.; Gareiss, P. C.; Tae, H. S.; Michel, J.; Noblin, D. J.; Jorgensen, W. L.; Ciulli, A.; Crews, C. M. Targeting the von Hippel-Lindau E3 ubiquitin ligase using small molecules to disrupt the VHL/HIF-1α interaction. J. Am. Chem. Soc. 2012, 134, 4465-4468.
(37) Frost, J.; Galdeano, C.; Soares, P.; Gadd, M. S.; Grzes, K. M.; Ellis, L.; Epemolu, O.; Shimamura, S.; Bantscheff, M.; Grandi, P.; Read, K. D.; Cantrell, D. A.; Rocha, S.; Ciulli, A. Potent and selective chemical probe of hypoxic signalling downstream of HIF-alpha hydroxylation via VHL inhibition. Nat Commun, 2016, 7, 13312-13312.
(38) Berlin, M.; Zimmerman, K.; Snyder, L.; Crew, A. P.; Crews, C. M.; Chen, X.; Dong, H. Q.; Ferraro, C.; Jin, M. H.; Qian, Y. M.; Siu, K.; Wang, J. Compounds and Methods for the Enhanced Degradation of Targeted Proteins. WO2016149668A1, Sep. 22, 2016.
(39) Ishoey, M.; Chorn, S.; Singh, N.; Jaeger, M. G.; Brand, M.; Paulk, J.; Bauer, S.; Erb, M. A.; Parapatics, K.; Müller, A. C.; Bennett, K. L.; Ecker, G. F.; Bradner, J. E.; Winter, G. E. Translation Termination Factor GSPT1 Is a Phenotypically Relevant Off-Target of Heterobifunctional Phthalimide Degraders. ACS Chem. Biol. 2018, 13, 553-560.
(40) Powell, C. E.; Gao, Y.; Tan, L.; Donovan, K. A.; Nowak, R. P.; Loehr, A.; Bahcall, M.; Fischer, E. S.; Jinne, P. A.; George, R. E.; Gray, N. S. Chemically Induced Degradation of Anaplastic Lymphoma Kinase (ALK). J. Med. Chem. 2018, 61, 4249-4255.
(41) Liu, V. W. S.; Yau, W. L.; Tam, C. W.; Yao, K. M.; Shiu, S. Y. W. Melatonin Inhibits Androgen Receptor Splice Variant-7 (AR-V7)-Induced Nuclear Factor-Kappa B (NF-κB) Activation and NF-κB Activator-Induced AR-V7 Expression in Prostate Cancer Cells: Potential Implications for the Use of Melatonin in Castration-Resistant Prostate Cancer (CRPC) Therapy. Int J Mol Sci. 2017, 18, E1130.
(42) Sun, H.; Nikolovska-Coleska, Z.; Lu, J.; Meagher, J. L.; Yang, C. Y.; Qiu, S.; Tomita, Y.; Ueda, Y.; Jiang, S.; Krajewski, K.; Roller, P. P.; Stuckey, J. A.; Wang, S. Design, synthesis, and characterization of a potent, nonpeptide, cell-permeable, bivalent Smac mimetic that concurrently targets both the BIR2 and BIR3 domains in XIAP. J. Am. Chem. Soc. 2007, 129, 15279-15294.
(43) Lu, J.; Bai, L.; Sun, H.; Nikolovska-Coleska, Z.; McEachern, D.; Qiu, S.; Miller, R. S.; Yi, H.; Shangary, S.; Sun, Y.; Meagher, J. L.; Stuckey, J. A.; Wang, S. SM-164: a novel, bivalent Smac mimetic that induces apoptosis and tumor regression by concurrent removal of the blockade of cIAP-1/2 and XIAP. Cancer Res. 2008, 68, 9384-9393.
(44) Bai, L.; Zhou, B.; Yang, C. Y.; Ji, J.; McEachern, D.; Przybranowski, S.; Jiang, H.; Hu, J.; Xu, F.; Zhao, Y.; Liu, L.; Fernandez-Salas, E.; Xu, J.; Dou, Y.; Wen, B.; Sun, D.; Meagher, J.; Stuckey, J.; Hayes, D. F.; Li, S.; Ellis, M. J.; Wang, S. Targeted Degradation of BET Proteins in Triple-Negative Breast Cancer. Cancer Res. 2017, 77, 2476-2487.
(45) Stols, L.; Gu, M.; Dieckman, L.; Raffen, R.; Collart, F. R.; Donnelly, M. I. A new vector for high-throughput, ligation-independent cloning encoding a tobacco etch virus protease cleavage site. Protein Expr Purif. 2002, 25, 8-15.
(46) Benoit, R. M.; Ostermeier, C.; Geiser, M.; Li, J. S.; Widmer, H.; Auer, M. Seamless Insert-Plasmid Assembly at High Efficiency and Low Cost. PLoS One. 2016, 11, e0153158.

It is to be understood that the foregoing embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I:
A-L-B
wherein:
A is a radical of an androgen receptor antagonist selected from the group consisting of: 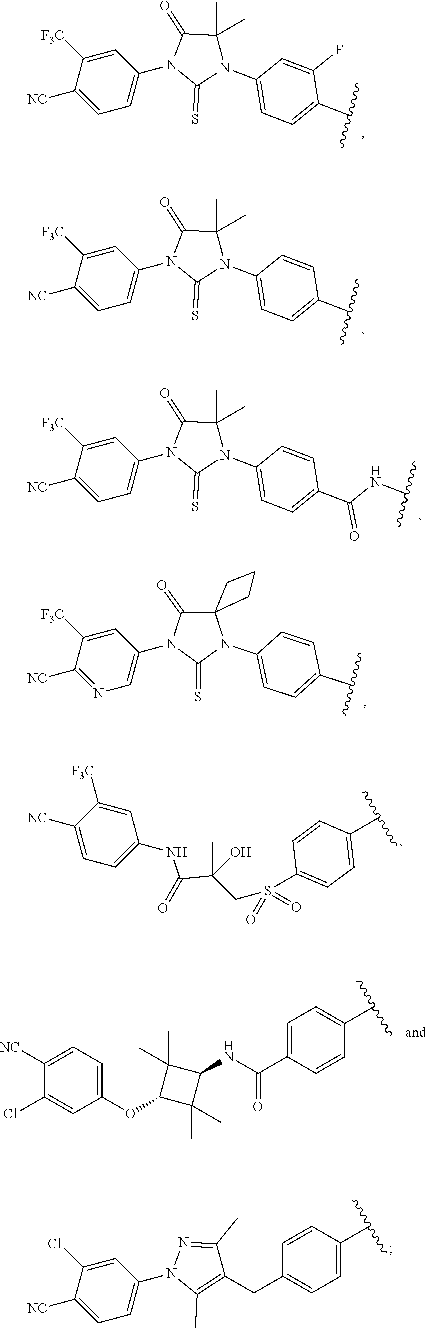
L is a linker; and
B is a radical of an E3 ligase ligand selected from the group consisting of: 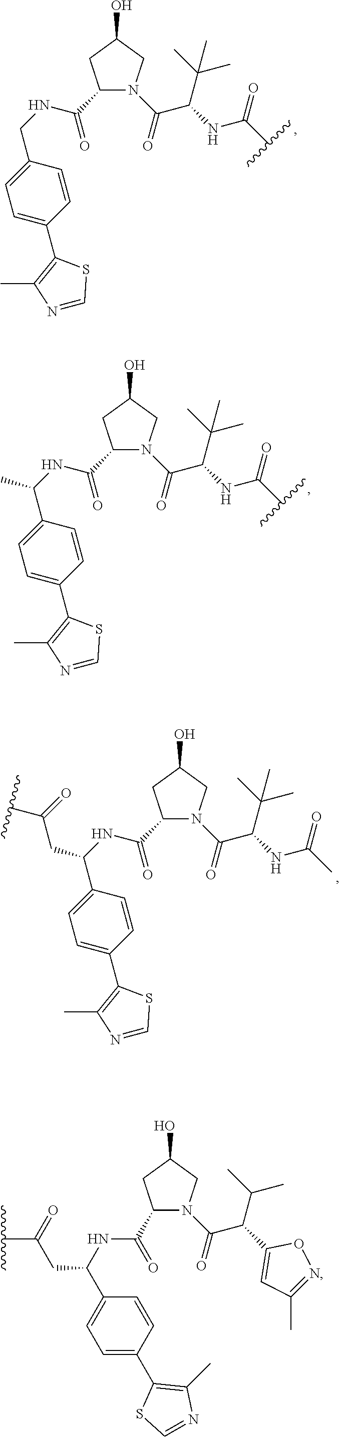 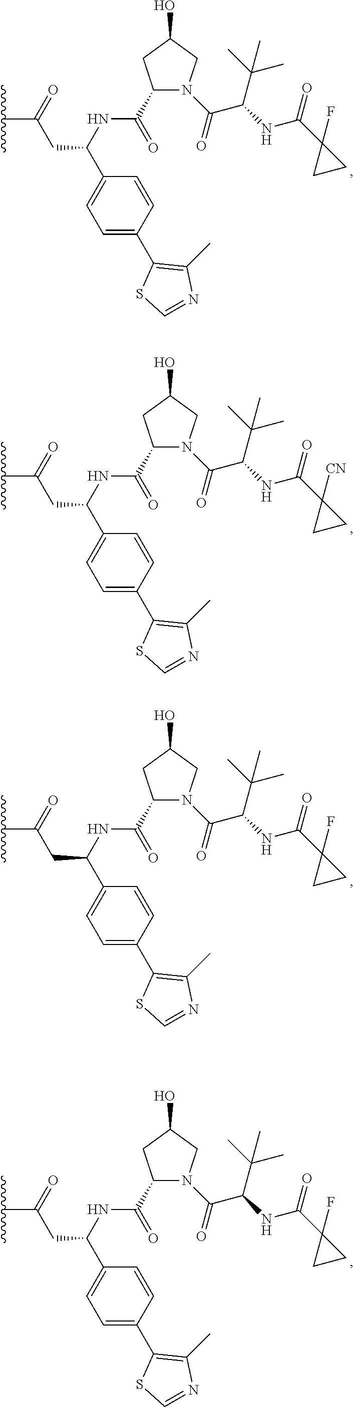 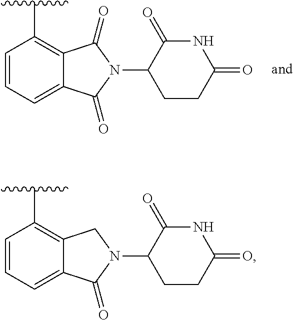
or a pharmaceutically acceptable salt or solvate thereof,
with the proviso the compound of Formula I is not (4R)-1-((S)-2-(2-(4-((4′-(3-(4-cyano-3-(trifluoromethyl)phenyl)-5,5-dimethyl-4-oxo-2-thioxoimidazolidin-1-yl)-[1,1′-biphenyl]-4-yl)oxy)butoxy)acetamido)-3,3-dimethylbutanoyl)-4-hydroxy-N-(4-(4-methylthiazol-5-yl)benzyl)pyrrolidine-2-carboxamide.

2. The compound of claim 1, wherein the radical of an androgen receptor antagonist is selected from the group consisting of: 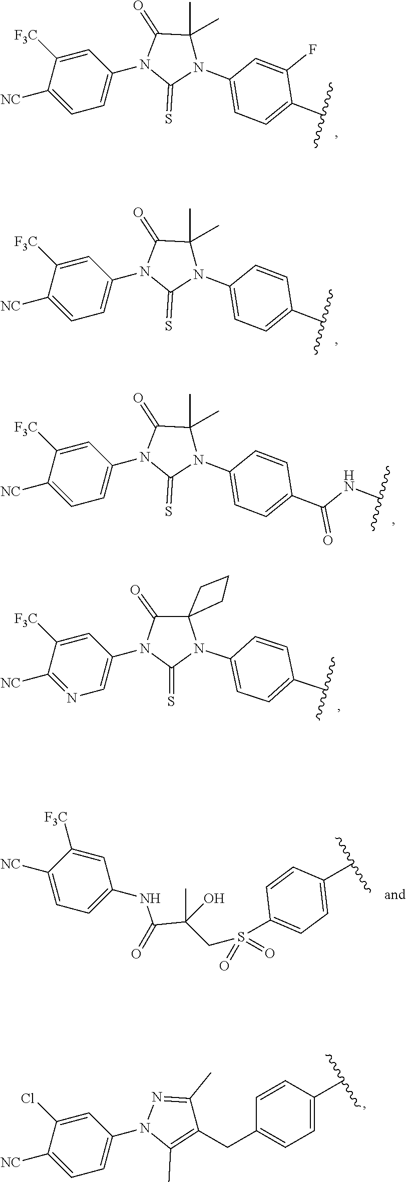
or a pharmaceutically acceptable salt or solvate thereof.

3. The compound of claim 1, wherein the radical of the androgen receptor antagonist is: 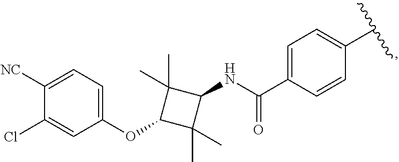
or a pharmaceutically acceptable salt or solvate thereof.

4. The compound of any one of claims 1-3, wherein the radical of an E3 ligase ligand is selected from the group consisting of: 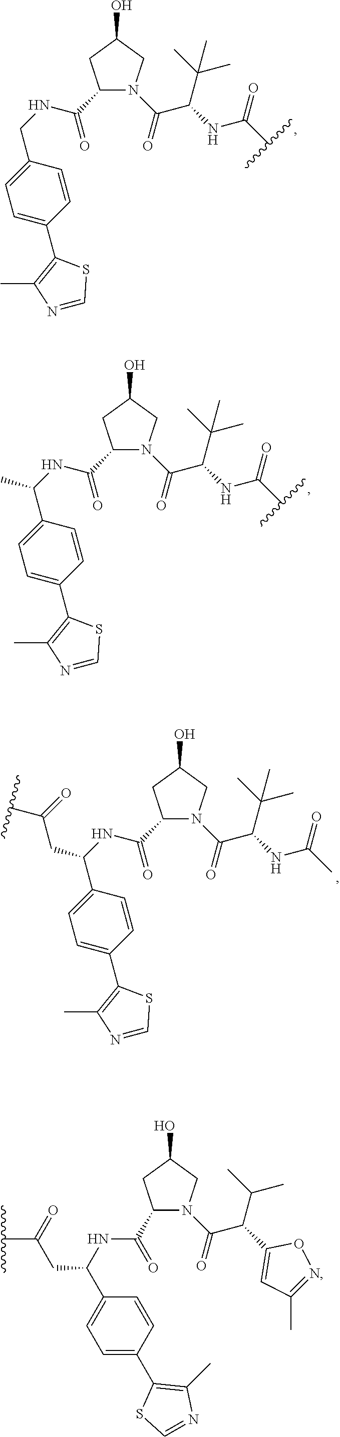 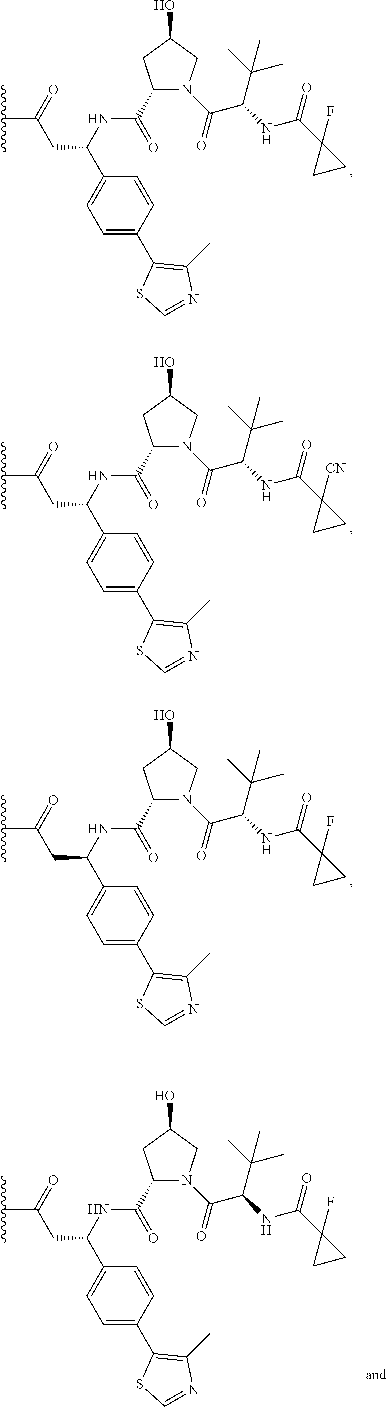 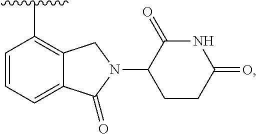
or a pharmaceutically acceptable salt or solvate thereof.

5. A compound having Formula II: 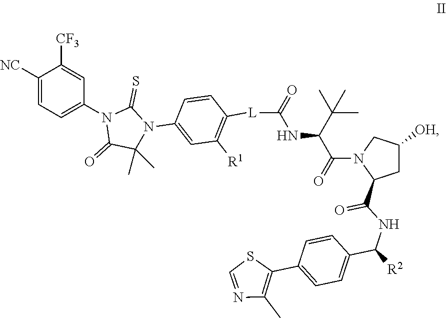
wherein:
L is a linker;
R¹ is selected from the group consisting of hydrogen and fluoro; and
R² is selected from the group consisting of hydrogen and C₁-C₃ alkyl,
or a pharmaceutically acceptable salt or solvate thereof.

6. The compound of claim 1 having Formula III: 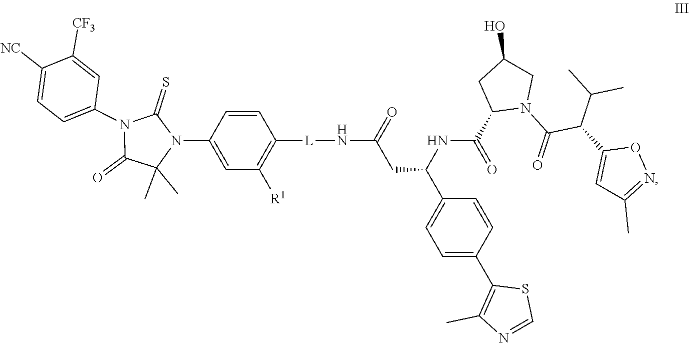
wherein:
R¹ is selected from the group consisting of hydrogen and fluoro,
or a pharmaceutically acceptable salt or solvate thereof.

7. The compound of any one of claims 1-6, wherein
L is —X-L¹-Z—;
X is selected from the group consisting of —C≡C—, —O—, —C(═O)N(R³)—, and —N(R⁴)—; or
X is absent;
Z is selected from the group consisting of —C≡C—, —O—, —C(═O)N(R³)—, and —N(R⁴)—; or
Z is absent;
L¹ is selected from the group consisting of alkylenyl, heteroalkylenyl, and —W¹—(CH₂)ₘ—W²—(CH₂)ₙ—
W¹ is absent; or
W¹ is selected from the group consisting of phenylenyl, heteroarylenyl, heterocyclenyl, and cycloalkylenyl;
W² is selected from the group consisting of phenylenyl, heteroarylenyl, heterocyclenyl, and cycloalkylenyl;
m is 0, 1, 2, 3, 4, 5, 6, or 7;
n is 0, 1, 2, 3, 4, 5, 6, 7, or 8;
R³ is selected from the group consisting of hydrogen and C₁₋₄ alkyl; and
R⁴ is selected from the group consisting of hydrogen and C₁₋₄ alkyl,
or a pharmaceutically acceptable salt or solvate thereof.

8. The compound of claim 7, wherein L is selected from the group consisting of: 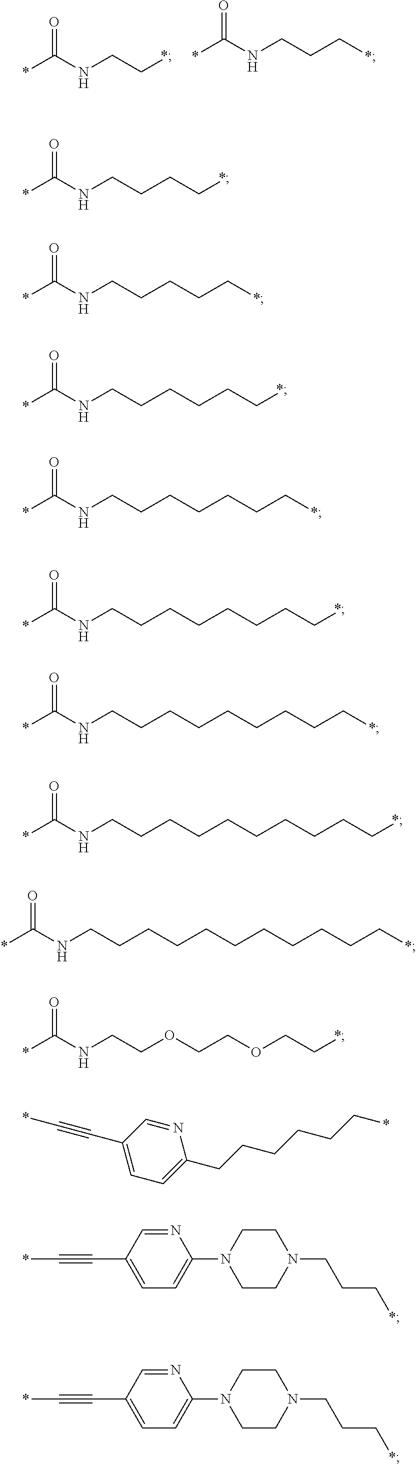 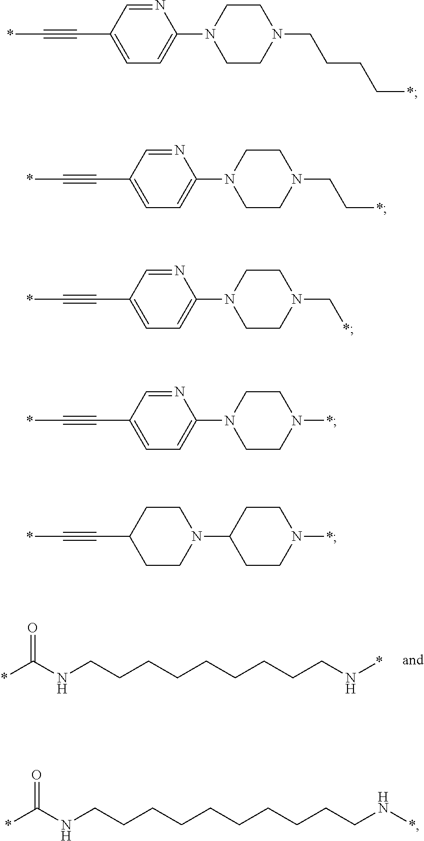
or a pharmaceutically acceptable salt or solvate thereof.

9. The compound of claim 1 having Formula IV: 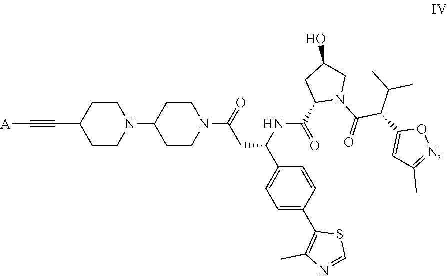
or a pharmaceutically acceptable salt or solvate thereof.

10. The compound of claim 9, wherein A is selected from the group consisting of: 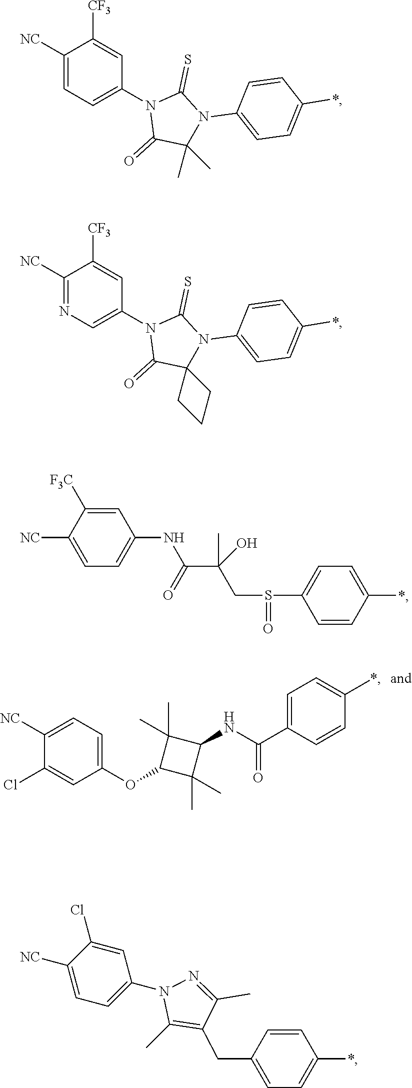
or a pharmaceutically acceptable salt or solvate thereof.

11. The compound of claim 1 having Formula V: 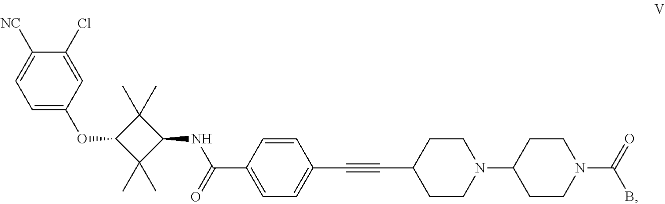
or a pharmaceutically acceptable salt or solvate thereof.

12. The compound of claim 11, wherein B is selected from the group consisting of: 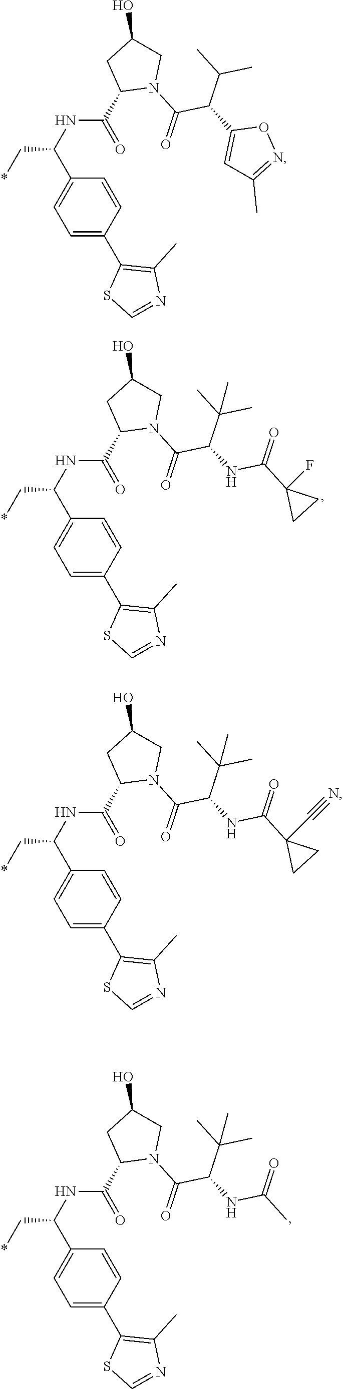 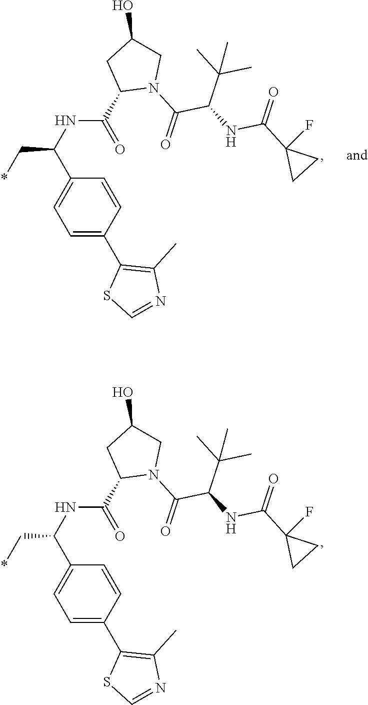 [SMILES: *C[C@@H](NC(=O)[C@@H]1C[C@@H](O)CN1C(=O)[C@@H](NC(=O)C1(F)CC1)C(C)(C)C)c1ccc(-c2scnc2C)cc1 ; *C[C@H](NC(=O)[C@@H]1C[C@@H](O)CN1C(=O)[C@H](NC(=O)C1(F)CC1)C(C)(C)C)c1ccc(-c2scnc2C)cc1]
or a pharmaceutically acceptable salt or solvate thereof.

13. A pharmaceutical composition comprising a compound of any one of claims 1-12, or a pharmaceutically acceptable salt or solvate thereof, and a pharmaceutically acceptable excipient.

14. A method of treating cancer in a patient in need thereof, the method comprising administering to the patient a pharmaceutically effective amount of a compound of any one of claims 1-12, or a pharmaceutically acceptable salt or solvate thereof.

15. The method of claim 14, wherein the cancer is prostate cancer.

16. The method of claim 15, wherein the cancer is castration-resistant prostate cancer.

17. The method of any one of claims 14-16, wherein the compound is administered in combination with a second anticancer agent.

18. The method of claim 17, wherein the anticancer agent is selected from the group consisting of enzalutamide, bicalutamide, abiraterone, nilutamide, flutamide, apalutamide, finasteride, dutasteride, or alfatradiol
